SEQUENCE LISTING (11 sequences)

SEQ ID NO: 1 - protein, 1035 aa, Artificial Sequence
  Synthetic Construct
MPEQERQITAREGASRKILSKLSLPTRAWEPAMKKSFAFDNVGYEGGLDGLGPSSQPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 2 - DNA, 3108 bp, Artificial Sequence
  Synthetic Construct
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 3 - DNA, 305 bp, Artificial Sequence
  Synthetic Construct
cgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 4 - DNA, 56 bp, Artificial Sequence
  Synthetic Construct
aataaagacctcttattttcattcatcaggtgtggttggtttttttgtgtgggggc

SEQ ID NO: 5 - DNA, 141 bp, Artificial Sequence
  Synthetic Construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 130 bp, Artificial Sequence
  Synthetic Construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 7 - DNA, 140 bp, Artificial Sequence
  Synthetic Construct
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 8 - DNA, 130 bp, Artificial Sequence
  Synthetic Construct
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 9 - DNA, 3817 bp, Artificial Sequence
  Synthetic Construct
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaagcggccgccaccatgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgaggtaccaataaagacctcttattttcattcatcaggtgtggttggtttttttgtgtgggggcggatccatcggatcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 10 - protein, 736 aa, Artificial Sequence
  Synthetic Construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 11 - DNA, 2211 bp, Artificial Sequence
  Synthetic Construct
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

(19) United States
(12) Patent Application Publication
(10) Pub. No.: US 2024/0307553 A1
(43) Pub. Date: Sep. 19, 2024

(54) PHARMACEUTICAL COMPOSITIONS CONTAINING ADENO-ASSOCIATED VIRAL VECTOR

(71) Applicant: Pfizer Inc., New York, NY (US)

(72) Inventors: Tamara Shafer HODGE, Ballwin, MO (US); Ekaterina Vasilievna TORRES, Eureka, MO (US)

(21) Appl. No.: 18/551,038

(22) PCT Filed: Mar. 29, 2022
(86) PCT No.: PCT/IB2022/052882

§ 371 Date: Sep. 18, 2023

Related U.S. Application Data
(60) Provisional application No. 63/268,676, filed on Feb. 28, 2022.
(60) Provisional application No. 63/169,428, filed on Apr. 1, 2021.

(51) Int. Cl. A61K 48/00; A61K 38/46; A61K 47/02; A61K 47/10; A61K 47/18; A61K 47/26; C12N 15/86

(52) U.S. Cl. CPC A61K 48/0033; A61K 38/46; A61K 47/02; A61K 47/10; A61K 47/18; A61K 47/26; A61K 48/0075; C12N 15/86; C12N 2750/14122; C12N 2750/14143

(57) ABSTRACT

The present invention provides compositions comprising a recombinant AAV (rAAV) vector and one or more pharmaceutically acceptable excipients. The compositions have improved stability as compared to other rAAV compositions.

### REFERENCE TO SEQUENCE LISTING

This application is being filed electronically via EFS-Web and includes an electronically submitted sequence listing in .txt format. The .txt file contains a sequence listing entitled “PC072723A_SequenceListing_ST25.txt” created on Mar. 7, 2022, and having a size of 29.3 KB. The sequence listing contained in this .txt file is part of the specification and is herein incorporated by reference in its entirety.

### BACKGROUND OF THE INVENTION

The present invention relates to the field of pharmaceutical formulations of adeno-associated viral (AAV) vectors, including recombinant AAV (rAAV) vectors. Specifically, the present invention relates to a formulation for the pharmaceutical preparation and use of a rAAV vector, and in particular a rAAV vector comprising an AAV3B capsid polypeptide.

Gene therapy, including those therapies that use a recombinant AAV (rAAV) vector to deliver a therapeutic transgene, has the potential to treat a wide range of serious diseases for which no cure, and in many cases, limited treatment exists (Wang et al. (2019) Nature Reviews 18:358-378). Gene therapy, using a rAAV vector, introduces a healthy copy of a defective gene to a patient which then expresses a protein with a normal structure or function.

rAAV vectors can be made using any of the naturally occurring, synthetic or chimeric serotypes of AAV. AAV3, which is closely related to the well characterized AAV2 serotype, was derived from a human source (Hoggan et al. (1966) PNAS 55:1467-1474). AAV3 was deposited with the American Type Culture Collection (ATCC, Manassas, VA) as AAV3 strain H and subsequently found to comprise two distinct isolates, AAV3A and AAV3B. These two isolates were later cloned and sequenced and six amino acid differences were found between the Cap proteins and five amino acid differences were found between the Rep proteins (Muramatsu et al. (1996) Virology 221:28-217; Rutledge et al. (1998) J. Virology 72:309-319). The amino acid sequence of the AAV3B VP1 polypeptide (GenBank accession no. AF028705.1) is 87.9% identical to the amino acid sequence of the AAV2 VP1 polypeptide (GenBank accession no. NC_001401.2). The nucleotide sequence of the AAV3B VP1 gene (GenBank accession no. AF028705.1) is 81% identical to the nucleotide sequence of the AAV2 VP1 gene (GenBank accession no. NC_001401.2). Confirmation that AAV3B is a unique serotype was shown by the inability of anti-AAV2 serum to block cell transduction by AAV3B (Rutledge et al. (1998) J. Virology 72:309-319). Recombinant AAV3B vectors have been shown to transduce primary human hepatocytes efficiently because AAV3B uses human hepatocyte growth factor receptor as a cellular receptor (Li et al., (2015) Mol. Ther. 23(12):1867-1876).

Wilson disease (WD) is a rare genetic disease with an incidence of about one in 30,000 and for which there is no cure, and thus requires life-long treatment. WD is a disorder of copper metabolism and storage which may present with hepatic, neurologic and/or psychiatric symptoms (Aggarwal and Bhatt (2013) Internat. Rev. Neurobiol. 110:313-348; Mohr and Weiss, (2019) Clin. Biochem. Rev. 40(2):59-77; Weiss, K. H. Gene Reviews ncbi.nlm.nih.gov/books/NBK1512/). Onset of symptoms in patients with WD typically occurs in the teenage years or in young adulthood and patients may present with jaundice, hepatitis, hepatic failure and chronic liver disease. Neurologic symptoms include movement disorders and/or dystonia. When copper levels in the body are high, WD patients will present with Kayser-Fleischer rings in the eyes.

WD is inherited as an autosomal recessive disease and is due to mutations (about 500 mutations have been identified) in the ATP7B gene (GenBank accession no. NM_000053.4) which encodes a copper transporting ATPase 2 (UniProtKB accession no. P35670). In the liver, which is the site of metabolism for dietary copper, copper-transporting ATPase 2 is responsible for the dual role of (i) regulating hepatocyte copper concentration by eliminating excess copper into the feces via the biliary route and (ii) transferring copper to copper-dependent enzymes, including circulating ceruloplasmin. Current treatments for WD rely primarily on life-long chelation modalities, dietary copper restriction and for those who do not respond to medical treatment or are unable to tolerate treatment due to side effects, liver transplantation. Due to the morbidity associated with current therapy, gene therapy offers a promising treatment and cure for WD.

### SUMMARY OF THE INVENTION

The present disclosure provides stable rAAV vector formulations suitable for clinical administration. Compositions comprising a rAAV vector, and in particular a rAAV vector comprising an AAV3B polypeptide, and excipients capable of maintaining the stability of the vector during extended storage, and under stress conditions, are provided. In one aspect, the disclosure provides a pharmaceutical composition comprising an rAAV vector, a buffer (e.g., Tris), a salt (e.g., magnesium chloride (MgCl₂)), a cryoprotectant (e.g., sucrose), and a poloxamer (e.g., poloxamer 188) having a pH of about 7.3 to about 7.9 and which is suitable for parenteral administration.

In another aspect, the disclosure provides a pharmaceutical composition comprising a recombinant adeno-associated virus (rAAV) vector, a buffer; a salt; a cryoprotectant; and a surfactant, wherein the pH of the pharmaceutical composition is from about 7.1 to about 8.1. In some embodiments, the concentration of the buffer is about 1 mM to about 100 mM. In some embodiments, the concentration of the buffer is about 20 mM. In some embodiments, the buffer is Tris. In some embodiments, the Tris is Tris base, Tris HCl or a combination of Tris base and Tris HCl. In some embodiments, the concentration of the salt is about 10 mM to about 200 mM. In some embodiments, the concentration of the salt is about 100 mM. In some embodiments, the salt is magnesium chloride (MgCl₂). In some embodiments, the concentration of the cryoprotectant is about 1% (w/v) to about 10% (w/v). In some embodiments, the concentration of the cryoprotectant is about 4% (w/v). In some embodiments, the cryoprotectant is sucrose. In some embodiments, the concentration of the surfactant is about 0.002% (w/v) to about 0.2% (w/v). In some embodiments, the concentration of the surfactant is about 0.02% (w/v). In some embodiments, the surfactant is poloxamer. In some embodiments, the poloxamer is poloxamer 188.

In some embodiments, the pharmaceutical composition comprises about 1E+11 vector genome (vg)/mL to about 1E+15 vg/mL or about 3.0E+11 vg/mL to about 3.0E+13 vg/mL of the rAAV vector. In some embodiments, the pharmaceutical composition comprises about 0.5E+13 vg/mL, about 0.6E+13 vg/mL, about 0.7E+13 vg/mL, about 0.8E+13 vg/mL, about 0.9E+13 vg/mL, about 1E+13 vg/mL, about 1.1E+13 vg/mL, about 1.2E+13 vg/mL, 1.3E+13 vg/mL, 1.4E+13 vg/mL about 1.5E+13 vg/mL, about 1.6E+13 vg/mL, about 1.7E+13 vg/mL, about 1.8E+13 vg/mL, about 1.9E+13 vg/mL, about 2.0E+13 vg/mL, about 2.1E+13 vg/mL, about 2.2E+13 vg/mL, about 2.3E+13 vg/mL, about 2.43E+13 vg/mL, about 2.5E+13 vg/mL, about 2.6E+13 vg/mL, about 2.7E+13 vg/mL, about 2.8E+13 vg/mL, about 2.9E+13 vg/mL or about 3.0E+13 vg/mL of the rAAV vector. In some embodiments, the pharmaceutical composition comprises about 4E+11 vg/mL, about 2E+12 vg/mL, about 1E+13 vg/mL or about 2E+13 of the rAAV vector.

In some embodiments, the pH of the pharmaceutical composition is about 7.3 to about 7.9, optionally about 7.6.

In some embodiments, the viscosity of the pharmaceutical composition is about 0.5 mPa to about 5 mPa. In some embodiments, the viscosity of the pharmaceutical composition is about 1 mPa to about 1.5 mPa, optionally about 1.19 mPa.

In some embodiments, the density of the pharmaceutical composition is about 0.5 g/cm³ to about 5 g/cm³. In some embodiments, the density of the pharmaceutical composition is about 1 g/cm³ to about 1.5 g/cm³, optionally about 1.03 g/cm³.

In some embodiments, the conductivity of the pharmaceutical composition is about 1 mS/cm to about 40 mS/cm. In some embodiments, the conductivity of the pharmaceutical composition is about 10 mS/cm to about 20 mS/cm, optionally about 16.9 mS/cm.

In some embodiments, the rAAV vector of the pharmaceutical composition comprises a capsid polypeptide of an AAV serotype selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1, RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15, optionally wherein the capsid polypeptide is of the AAV3B serotype. In some embodiments, the capsid polypeptide of the AAV3B serotype comprises or consists of the amino acid sequence of SEQ ID NO:10.

In some embodiments, the rAAV vector of the pharmaceutical composition comprises a vector genome comprising an ATP7B transgene, or fragment thereof. In some embodiments, the ATP7B transgene i) comprises or consists of the nucleic acid sequence of SEQ ID NO:2, ii) encodes a copper transporting ATPase 2 with a deletion of metal binding sites (MBS) 1-4, iii) encodes a copper transporting ATPase 2 comprising or consisting of the amino acid sequence of SEQ ID NO:1 or iv) a combination thereof. In some embodiments, the rAAV vector genome further comprises one or more elements for the expression of the ATP7B transgene. In some embodiments, the one or more elements include an AAT promoter, a polyA signal sequence and flanking 5′ and 3′ ITR sequences. In some embodiments, the ITR sequences are of the AAV2 serotype. In some embodiments, the rAAV vector comprises a vector genome comprising or consisting of the nucleic acid sequence of SEQ ID NO:9.

In another aspect, the disclosure provides a pharmaceutical composition comprising a recombinant adeno-associated virus (rAAV) vector; about 20 mM Tris; about 100 mM MgCl₂; about 4% (w/v) sucrose; and about 0.02% (w/v) poloxamer 188, wherein the pH of composition is from about 7.1 to about 8.1. In some embodiments, the composition comprises about 3.0E+11 vg/mL to about 3.0E+13 vg/mL of the rAAV vector. In some embodiments, the composition comprises about 4E+11 vg/mL, about 2E+12 vg/mL, about 1E+13 vg/mL or about 2E+13 vg/mL of the rAAV vector. In some embodiments, the rAAV vector of the pharmaceutical composition comprises a polypeptide of the AAV3B serotype, optionally, wherein the polypeptide comprises or consists of the amino acid sequence of SEQ ID NO:10. In some embodiments, the rAAV vector comprises a vector genome comprising a transgene and one or more elements, optionally, wherein the transgene is ATP7B, or a fragment thereof, and optionally, wherein the ATP7B transgene comprises a nucleic acid encoding a copper-transporting ATPase 2 with a deletion of metal binding sites (MBS) 1-4, comprises a nucleic encoding a copper transporting ATPase 2 comprising the amino acid sequence of SEQ ID NO:1 or both. In some embodiments, the one or more elements include an AAT promoter, a polyA signal sequence and flanking 5′ and 3′ ITR sequences. In some embodiments, the ITR sequences are of the AAV2 serotype. In some embodiments, the rAAV vector comprises a vector genome comprising or consisting of a nucleic acid comprising a nucleic acid encoding a copper transporting ATPase 2 comprising the amino acid sequence of SEQ ID NO:1, a promoter comprising the nucleic acid sequence of SEQ ID NO:3, a polyA signal sequence comprising the nucleic acid sequence of SEQ ID NO:4 and a 5′ ITR and a 3′ ITR, optionally comprising the sequence of any one of SEQ ID NO:5-8.

In another aspect, the disclosure provides a pharmaceutical composition comprising a recombinant adeno-associated virus (rAAV) vector comprising a capsid polypeptide of the AAV3B serotype; about 14 mM to about 26 mM, optionally about 20 mM Tris; about 70 mM to about 130 mM, optionally about 100 mM MgCl₂; about 2.8% (w/v) to about 5.2% (w/v), optionally about 4% (w/v) sucrose; and about 0.014% (w/v) to about 0.26% (w/v), optionally about 0.02% (w/v) poloxamer 188, wherein the pH of composition is from about 7.1 to about 8.1. In some embodiments, the rAAV vector of the pharmaceutical composition comprises a vector genome comprising a transgene. In some embodiments, the transgene i) comprises or consists of the nucleic acid sequence of SEQ ID NO:2, ii) encodes a copper transporting ATPase 2 with a deletion of metal binding sites (MBS) 1-4, iii) encodes a copper transporting ATPase 2 comprising or consisting of the amino acid sequence of SEQ ID NO:1 or iv) a combination thereof. In some embodiments, the vector genome comprises at least one element selected from the group consisting of a promoter, a polyA signal sequence, at least one ITR and a combination thereof. In some embodiments, the promoter comprises or consists of a nucleic acid sequence of SEQ ID NO:3. In some embodiments, the polyA signal sequence comprises or consists of a nucleic acid sequence of SEQ ID NO:4. In some embodiments, the ITR sequence comprises or consists of a nucleic acid sequence of any one of SEQ ID NO:5-8. In some embodiments, the vector genome comprises or consists of a nucleic acid comprising a nucleic acid encoding a copper transporting ATPase 2 comprising the amino acid sequence of SEQ ID NO:1, a promoter comprising the nucleic acid sequence of SEQ ID NO:3, a polyA signal sequence comprising the nucleic acid sequence of SEQ ID NO:4 and a 5′ ITR and a 3′ ITR, optionally comprising the sequence of any one of SEQ ID NO:5-8.

In another aspect, the disclosure provides a pharmaceutical composition comprising a recombinant adeno-associated virus (rAAV) vector comprising a capsid polypeptide of the AAV3B serotype and a vector genome comprising a transgene encoding a copper transporting ATPase 2 protein or fragment thereof; about 20 mM Tris; about 100 mM MgCl₂; about 4% (w/v) sucrose; and about 0.02% (w/v) poloxamer 188, wherein the pH of composition is from about 7.1 to about 8.1. In some embodiments, the transgene i) comprises or consists of the nucleic acid sequence of SEQ ID NO:2, ii) encodes a copper transporting ATPase 2 with a deletion of metal binding sites (MBS) 1-4, iii) encodes a copper transporting ATPase 2 comprising or consisting of the amino acid sequence of SEQ ID NO:1 or iv) a combination thereof. In some embodiments, the vector genome comprises at least one element selected from the group consisting of a promoter, a polyA signal sequence, at least one ITR and a combination thereof. In some embodiments, the promoter comprises or consists of a nucleic acid sequence of SEQ ID NO:3. In some embodiments, the polyA signal sequence comprises or consists of a nucleic acid sequence of SEQ ID NO:4. In some embodiments, the ITR sequence comprises or consists of a nucleic acid sequence of any one of SEQ ID NO:5-8. In some embodiments, the vector genome comprises or consists of a nucleic acid comprising a nucleic acid encoding a copper transporting ATPase 2 comprising the amino acid sequence of SEQ ID NO:1, a promoter comprising the nucleic acid sequence of SEQ ID NO:3, a polyA signal sequence comprising the nucleic acid sequence of SEQ ID NO:4 and a 5′ ITR and a 3′ ITR, optionally comprising the sequence of any one of SEQ ID NO:5-8.

In another aspect, the disclosure provides a pharmaceutical composition comprising a recombinant adeno-associated virus (rAAV) vector comprising a capsid polypeptide of the AAV3B serotype and a vector genome comprising a transgene encoding a copper transporting ATPase 2 comprising or consisting of the amino acid sequence of SEQ ID NO:1; about 20 mM Tris; about 100 mM MgCl₂; about 4% (w/v) sucrose; and about 0.02% (w/v) poloxamer 188, wherein the pH of composition is from about 7.1 to about 8.1. In some embodiments, the vector genome comprises at least one additional element selected from the group consisting of a promoter comprising or consisting of the nucleic acid sequence of SEQ ID NO:3, a polyA signal sequence comprising or consisting of the nucleic acid sequence of SEQ ID NO:4, at least one ITR sequence comprising or consisting of the nucleic acid sequence of any one of SEQ ID NO:5-8 and a combination thereof. In some embodiments, the vector genome comprises or consists of a nucleic acid comprising a nucleic acid encoding a copper transporting ATPase 2 comprising the amino acid sequence of SEQ ID NO:1, a promoter comprising the nucleic acid sequence of SEQ ID NO:3, a polyA signal sequence comprising the nucleic acid sequence of SEQ ID NO:4 and a 5′ ITR and a 3′ ITR, optionally comprising the sequence of any one of SEQ ID NO:5-8.

In some aspects, the disclosure provides a pharmaceutical composition is lyophilized or is not lyophilized. In some aspects, the disclosure provides, a vial comprises 0.5 mL to 10 mL, of a pharmaceutical composition disclosed herein. In some embodiments, the vial is made of cyclic-olefin copolymer. In some embodiments, the vial has an in-place thermoplastic elastomer stopper.

In some aspects, the disclosure provides a method of treating a disease comprising administering an effective amount of a pharmaceutical composition disclosed herein to a human subject having the disease. In some embodiments, the pharmaceutical composition comprises about 4E+11 vg/mL, about 2E+12 vg/mL, about 1E+13 vg/mL or about 2E+13 vg/mL of the rAAV vector. In some embodiments, the pharmaceutical composition is administered intravenously. In some embodiments the effective amount of the pharmaceutical composition is about 5E+11 vg/kg to about 1E+14 vg/kg. In some embodiments, the effective amount of the pharmaceutical composition is about 1E+12 vg/kg or about 1+E13 vg/kg. In some embodiments, the disease is due to a deficiency or dysfunction of copper-transporting ATPase 2. In some embodiments, the disease is Wilson disease.

In some aspects, the disclosure provides a method of reducing hepatic copper in a human subject in need thereof, comprising administering intravenously to the human subject a pharmaceutical composition disclosed herein, or the contents of the vial comprising a pharmaceutical composition disclosed herein. In some embodiments, the human subject has Wilson disease.

In some aspects, the disclosure provides a method of treating Wilson disease in a human subject in need thereof, comprising administering intravenously to a human subject a pharmaceutical composition disclosed herein, or the contents of the vial comprising a pharmaceutical composition disclosed herein.

In some aspects, the disclosures provides for the use of a pharmaceutical composition disclosed herein in the manufacture of a medicament for the treatment of a disease. In embodiments, the disease is Wilson disease.

In some aspects, the disclosure provides for the use of a pharmaceutical composition disclosed herein in the manufacture of a medicament for treatment of a disease due to deficiency or dysregulation of copper-transporting ATPase 2. In some embodiments, the disease is Wilson disease.

In some aspects, the disclosure provides a pharmaceutical composition disclosed herein for use in a method of treatment of a human subject. In some embodiments, the disease is Wilson disease.

In some aspects, the disclosure provides a pharmaceutical composition disclosed herein for use in a method of treating a human subject with a disease due to deficiency or dysregulation of copper-transporting ATPase 2. In some embodiments, the disease is Wilson disease.

In some aspects, the disclosure provides a kit comprising a pharmaceutical composition disclosed herein, or at least one vial comprising a pharmaceutical composition disclosed herein, and instructions for use.

### BRIEF DESCRIPTION OF THE FIGURES

FIG. 1 depicts exemplary transgene titer (vg/mL) of a pharmaceutical composition comprising a rAAV vector measured by qPCR following storage of the pharmaceutical composition for up to 6 months at 5° C., −20° C. and −70° C.

FIG. 2 depicts exemplary viral particle titer (vp/mL) of a pharmaceutical composition comprising a rAAV vector measured by size exclusion high performance liquid chromatography (SE-HPLC) following storage of the pharmaceutical composition for up to 6 months at 5° C., −20° C. and −70° C.

FIG. 3 depicts exemplary percentage of high molecular mass species (HMMS) of a pharmaceutical composition comprising a rAAV vector measured by SE-HPLC following storage of the pharmaceutical composition for up to 6 months at 5° C., −20° C. and −70° C.

FIG. 4 depicts exemplary empty capsid to full capsid ratio of a pharmaceutical composition comprising a rAAV vector measured by A260/A280, SE-HPLC following storage of the pharmaceutical composition for up to 6 months at 5° C., −20° C. and −70° C.

FIG. 5 depicts exemplary vector purity (% monomer) of a pharmaceutical composition comprising a rAAV vector measured by SE-HPLC following storage of the pharmaceutical composition for up to 6 months at 5° C., −20° C. and −70° C.

FIG. 6 depicts vector infectivity of a pharmaceutical composition comprising a rAAV vector measured as Median Tissue Culture Infectious Dose (TCID50) following storage of the pharmaceutical composition for up to 6 months at 5° C., −20° C. and −70° C.

FIG. 7 depicts exemplary pH of a pharmaceutical composition comprising a rAAV vector following storage of the pharmaceutical composition for up to 6 months at 5° C., −20° C. and −70° C.

FIG. 8 depicts exemplary capsid percent purity of a pharmaceutical composition comprising a rAAV vector measured by capillary gel electrophoresis, reducing (rCGE) following storage of the pharmaceutical composition for up to 6 months at 5° C., −20° C. and −70° C.

FIG. 9 depicts exemplary Z-average (nm) of a pharmaceutical composition comprising a rAAV vector measured by dynamic light scattering (DLS) following storage of the pharmaceutical composition for up to 6 months at 5° C., −20° C. and −70° C.

FIG. 10 depicts exemplary sub-visible particle count of a pharmaceutical composition comprising a rAAV vector measured by mean fluorescent intensity (MFI) following storage of the pharmaceutical composition for up to 6 months at 5° C.

FIG. 11 depicts exemplary capsid titer (VP/mL) and yield (%) for relatively empty rAAV3B capsids (top panel) and relatively full rAAV3B capsids (bottom panel) in various buffer solutions at time 0 (T0) and time 2 weeks (T2wk) at 5° C.

FIG. 12 depicts exemplary capsid yield (%) for relatively empty and relatively full rAAV3B capsids in various buffer solutions at time 0 (T0).

### DETAILED DESCRIPTION OF THE INVENTION

The present disclosure provides pharmaceutical compositions comprising AAV vectors and one or more pharmaceutically acceptable excipients. The present AAV vector compositions may comprise a rAAV whose vector genome carries a recombinant nucleic acid for expression of a protein of interest (e.g., a therapeutic protein). The present inventors have developed formulations of AAV vectors comprising a buffer, a salt, a cryoprotectant and a surfactant. Beneficially, these formulations stabilize a rAAV vector in the formulation for an extended period of time and under stress conditions (e.g., sheer, freeze/thaw). The inventors have also discovered that such formulations comprising a rAAV vector, and in particular a rAAV vector comprising an AAV3B capsid polypeptide, maintain product quality attributes at acceptable levels, including appearance, transgene titer, viral particle titer, percentage of high molecular mass species, empty to full capsid ratio, viral purity, infectivity, capsid purity, aggregation and pH during extended storage (e.g., 6 months) and under stress conditions (e.g., sheer, freeze/thaw).

### I. Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise. Numeric ranges are inclusive of the numbers defining the ranges. The terms “comprising,” “comprise,” “comprises,” “including” and “having” are intended to be inclusive and mean that there may be additional elements other than the listed elements. It is understood that wherever embodiments are described herein with the language “comprising,” otherwise analogous embodiments described in terms of “consisting of” and/or “consisting essentially of” are also provided. The following terms have the meanings given:

As used herein, the term “A260/A280” or “A260/A280 ratio” refers to the ratio of an absorbance measured at 260 nm and an absorbance measured at 280 nm. An A260/A280 ratio of a solution comprising rAAV capsids (e.g., a formulated drug substance) provides an estimation of the relative amounts of capsids with packaged DNA (i.e., full capsids and intermediate capsids) and without packaged DNA (i.e., empty capsids) that are present in the solution. For example, the higher the value, the greater the percentage of full and intermediate capsids present in the solution, such as a pharmaceutical formulation. Comparison of A260/A280 ratios between solutions allows for a relative estimation of capsid species, such that a solution with a higher A260/A280 has a greater percentage of full and intermediate capsids than a solution with a lower A260/A280 ratio. In some embodiments, an absorbance is measured using analytical size exclusion chromatography (SEC) in a high-performance liquid chromatography (HPLC) system, and measurement of the absorbance may be at one or more wavelengths (e.g., 260 nm and/or 280 nm).

As used herein, the term “about,” or “approximately” is used to indicate that a value includes the standard deviation of error for the device or method being employed to determine the value. In some embodiments, the term “about” can be added to any numeral recited herein to the extent the numeral would have a standard deviation of error when measuring.

As used herein, the term “and/or” refers to and encompasses any and all possible combinations of one or more of the associated listed items, as well as the lack of combinations when interpreted in the alternative (“or”).

As used herein, the term “chimeric” refers to a viral capsid or particle, with capsid or particle sequences from different parvoviruses, preferably different AAV serotypes, as described in Rabinowitz et al., U.S. Pat. No. 6,491,907, the disclosure of which is incorporated in its entirety herein by reference. See also Rabinowitz et al. (2004) J. Virol. 78(9):4421-4432. In some embodiments, a chimeric viral capsid is an AAV2.5 capsid which has the sequence of the AAV2 capsid with the following mutations: 263 Q to A; 265 insertion T; 705 N to A; 708 V to A; and 716 T to N. The nucleotide sequence encoding such capsid is defined as SEQ ID NO: 15 as described in WO 2006/066066. Other preferred chimeric AAV capsids include, but are not limited to, AAV2i8 described in WO 2010/093784, AAV2G9 and AAV8G9 described in WO 2014/144229, and AAV9.45 (Pulicherla et al. (2011) Molecular Therapy 19(6):1070-1078), AAV-NP4, NP22 and NP66, AAV-LK0 through AAV-LK019 described in WO 2013/029030, RHM4-1 and RHM15_1 through RHM5_6 described in WO 2015/013313, AAVDJ, AAVDJ/8, AAVDJ/9 described in WO 2007/120542.

As used herein, an “effective dosage” or “effective amount” of drug, compound, or pharmaceutical composition is an amount sufficient to effect any one or more beneficial or desired results. In more specific aspects, an effective amount prevents, alleviates or ameliorates symptoms of disease, and/or prolongs the survival of the subject being treated. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing one or more symptoms of a disease. The term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

As used herein, the term “formulation” or “composition” as they relate to a rAAV vector are meant to describe the rAAV vector in combination with a pharmaceutically acceptable excipient comprising at least one buffer, at least one salt (preferably that contributes a divalent cation), at least one cryoprotectant and at least one surfactant, wherein the pH is as defined. A “pharmaceutical formulation” or “pharmaceutical composition” refers to preparations which are in such form as to permit the biological activity of the active ingredients to be effective.

As used herein, the term “fragment” refers to a material or entity that has a structure that includes a discrete portion of the whole but lacks one or more moieties found in the whole. In some embodiments, a fragment consists of a discrete portion. In some embodiments, a fragment consists of or comprises a characteristic structural element or moiety found in the whole. In some embodiments, a polymer fragment comprises, or consists of, at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, 600, 700, 800, 900, 1000, 1100, 1200, 1300, 1400, 1500, 2000, 2500, 3000, 3500, 4000 or more (including ranges therein) monomeric units (e.g., amino acid residues, nucleotides) found in the whole polymer.

In some embodiments, a fragment is a fragment of a copper-transporting ATPase 2 polypeptide. In some embodiments, a fragment is a copper-transporting ATPase 2 polypeptide with a deletion of about 420 amino acids to about 440 amino acids (e.g., about 430 amino acids). In some embodiments, a fragment is a copper-transporting ATPase 2 polypeptide fragment comprises about 1020 amino acids to about 1050 amino acids (e.g., about 1035 amino acids). In some embodiments, a fragment of a copper-transporting ATPase 2 polypeptide does not include the metal binding sites (MBS) sites MBS1, MBS2, MBS3 and MBS4. In some embodiments, a fragment of a copper-transporting ATPase 2 polypeptide has a deletion of at least one MBS region, but retains MBS5 and MBS6.

In some embodiments, a fragment is a fragment of an APT7B gene. In some embodiments, a fragment is an ATP7B gene with a deletion of about 1280 nucleotides to about 1300 nucleotides (e.g., about 1290 nucleotides). In some embodiments, a fragment of an ATP7B gene comprises about 3090 nucleotides to about 3020 nucleotides (e.g., about 3105 nucleotides).

rAAV vectors are referred to as “full,” a “full capsid,” a “full vector” or a “fully packaged vector” when the capsid contains a complete vector genome, including a transgene. During production of rAAV vectors by host cells, vectors may be produced that have less packaged nucleic acid than the full capsids and contain, for example a partial or truncated vector genome. These vectors are referred to as “intermediates,” an “intermediate capsid,” a “partial” or a “partially packaged vector.” An intermediate capsid may also be a capsid with an intermediate sedimentation rate, that is a sedimentation rate between that of full capsids and empty capsids, when analyzed by analytical ultracentrifugation. Host cells may also produce viral capsids that do not contain any detectable nucleic acid material. These capsids are referred to as “empty(s),” or “empty capsids.” Full capsids may be distinguished from empty capsids based on A260/A280 ratios determined by SEC-HPLC, whereby the A260/A280 ratios have been previously calibrated against capsids (i.e., full, intermediate and empty) analyzed by analytical ultracentrifugation. Other methods known in the art for the characterization of capsids include CryoTEM, capillary isoelectric focusing and charge detection mass spectrometry. Calculated isoelectric points of ˜6.2 and ˜5.8 for empty and full AAV9 capsids, respectively have been reported (Venkatakrishnan et al., (2013) J. Virology 87.9:4974-4984).

As used herein, the term “gene” refers to a polynucleotide containing at least one open reading frame that is capable of encoding a particular polypeptide or protein after being transcribed and translated. “Gene transfer” or “gene delivery” refers to methods or systems for reliably inserting foreign DNA into host cells. Such methods can result in transient expression of non-integrated transferred DNA, extrachromosomal replication and expression of transferred replicons (e.g. episomes), and/or integration of transferred genetic material into the genomic DNA of host cells.

As used herein, the term “identity” or “identical to” refers to the overall relatedness between polymeric molecules, e.g., between nucleic acid molecules (e.g., DNA molecules and/or RNA molecules) and/or between polypeptide molecules. “Identity” measures the percent of identical matches between two or more sequences with gap alignments addressed by a particular mathematical model of computer programs (i.e. “algorithms”).

In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98% or 99% identical.

Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, sequences can be aligned using the methods and computer programs, including BLAST, available over the world wide web at ncbi.nlm.nih.gov/BLAST/. Other alignment programs include MegAlign® program in the Lasergene® suite of bioinformatics software (DNASTAR®, Inc., Madison, WI). Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Also, of interest is the BestFit program using the local homology algorithm of Smith and Waterman (1981, Advances in Applied Mathematics 2: 482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, WI, USA. Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc.

As used herein, the term “infectivity ratio” or “IR” refers to the number of rAAV vector particles needed to infect a cell. In some embodiments, the cell is in an in vitro system. In some embodiments, the cell is a cell within, or taken from, a subject in need of treatment with the rAAV vector. Infectivity ratio may be measured by any method known in the art including a cell-based qPCR assay. Infectivity may be measured as a median Tissue Culture Infectious dose (TCID50). Infectivity may be expressed as infectivity units (IU) per volume, IU/mL, or relative to the amount of vg present, IU/vg.

As used herein, the terms “inverted terminal repeat, “ITR,” “terminal repeat,” and “TR” refer to palindromic terminal repeat sequences at or near the ends of the AAV virus genome, comprising mostly complementary, symmetrically arranged sequences. These ITRs can fold over to form T-shaped hairpin structures that function as primers during initiation of DNA replication. They are also needed for viral genome integration into host genome, for the rescue from the host genome; and for the encapsidation of viral nucleic acid into mature virions. The ITRs are required in cis for vector genome replication and its packaging into viral particles. “5′ ITR” refers to the ITR at the 5′ end of the AAV genome and/or 5′ to a recombinant transgene. “3′ ITR” refers to the ITR at the 3′ end of the AAV genome and/or 3′ to a recombinant transgene. Wild-type ITRs are approximately 145 bp in length. A modified, or recombinant ITR, may comprise a fragment or portion of a wild-type AAV ITR sequence. One of ordinary skill in the art will appreciate that during successive rounds of DNA replication ITR sequences may swap such that the 5′ ITR becomes the 3′ ITR, and vice versa. In some embodiments, at least one ITR is present at the 5′ and/or 3′ end of a recombinant vector genome such that the vector genome can be packaged into a capsid to produce a rAAV vector (also referred to herein as “rAAV vector particle” or “rAAV viral particle”) comprising the vector genome.

As used here, the term “nucleic acid construct,” refers to a non-naturally occurring nucleic acid molecule resulting from the use of recombinant DNA technology (e.g., a recombinant nucleic acid). A nucleic acid construct is a nucleic acid molecule, either single or double stranded, which has been modified to contain segments of nucleic acid sequences, which are combined and arranged in a manner not found in nature. A nucleic acid construct may be a “vector” (e.g., a plasmid, a rAAV vector genome, an expression vector, etc.), that is, a nucleic acid molecule designed to deliver exogenously created DNA into a host cell.

As used herein, the term “percent purity,” “ % purity” refers to a purity of a solution comprising intact capsids, i.e., full capsids, empty capsids and intermediate capsids. A percent purity is determined by methods known in the art, including reverse phase HPLC, non-reducing conditions (RP-HPLC, NR). The area under the chromatogram absorbance curve generated by RP-HPLC, NR that is attributable to capsids present in the solution, is expressed as a percentage of the total area under the absorbance curve. Percent purity may also be measured by SDS-PAGE or capillary electrophoresis.

As used herein, the term “percent capsid purity” or “ % capsid purity” refers assessment of the purity of the individual capsid polypeptides, i.e., VP1, VP2 and VP3. In some embodiments, purity of each of VP1, VP2 and VP3 is assessed under reducing conditions by CGE. In some embodiments, purity of each of VP1, VP2 and VP3 is assessed under reducing conditions by RP-HPLC.

As used herein, the term “pharmaceutically acceptable” and “physiologically acceptable” refers to a biologically acceptable formulation, gaseous, liquid or solid, or mixture thereof, which is suitable for one or more routes of administration, in vivo delivery or contact. “Pharmaceutically acceptable excipients” (vehicles, additives) are those, which can safely be administered to a subject to provide an effective dose of the active ingredient employed. The term “excipient” or “carrier” as used herein refers to an inert substance, which is commonly used as a diluent, vehicle, preservative, binder or stabilizing agent for drugs. As used herein, the term “diluent” refers to a pharmaceutically acceptable (safe and non-toxic for administration to a human) solvent and is useful for the preparation of the liquid formulations herein. Exemplary diluents include, but are not limited to, sterile water and bacteriostatic water for injection (BWFI).

As used herein, the term “polynucleotide” or “nucleic acid” refers to a polymeric form of nucleotides, either ribonucleotides or deoxynucleotides, or a modified form of either type of nucleotide, and may be single or double stranded forms. A “polynucleotide” or a “nucleic acid” sequence encompasses its complement unless otherwise specified. As used herein, the term “isolated polynucleotide,” “isolated nucleic acid” or “isolated recombinant nucleic acid” means a polynucleotide of genomic, cDNA, or synthetic origin, or some combination thereof, which by virtue of its origin or source of derivation, has one to three of the following: (1) is not associated with all or a portion of a polynucleotide with which the “isolated polynucleotide” is found in nature, (2) is operably linked to a polynucleotide to which it is not linked in nature, or (3) does not occur in nature as part of a larger sequence.

As used herein, the term “recombinant,” refers to a vector, polynucleotide (e.g., a recombinant nucleic acid), polypeptide or cell that is the product of various combinations of cloning, restriction or ligation steps (e.g., relating to a polynucleotide or polypeptide comprised therein), and/or other procedure that results in a construct that is distinct from a product found in nature. A recombinant virus or vector (e.g., rAAV vector) comprises a vector genome comprising a recombinant nucleic acid (e.g., a nucleic acid comprising a transgene and one or more regulatory elements for the expression of the transgene). The terms respectively include replicates of the original polynucleotide construct and progeny of the original virus construct.

As used herein, the term “subject” refers to an organism, for example, a mammal (e.g., a human, a non-human mammal, a non-human primate, a primate, a laboratory animal, a mouse, a rat, a hamster, a gerbil, a cat, a dog). In some embodiments, a human subject is an adult, adolescent, or pediatric subject. In some embodiments, a subject is suffering from a disease, disorder or condition, e.g., a disease, disorder or condition that can be treated as provided herein. In some embodiments, a subject is suffering from a disease, disorder or condition associated with deficient or dysfunctional copper-transporting ATPase 2, e.g., Wilson disease. In some embodiments, a subject is susceptible to a disease, disorder, or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a human patient. In some embodiments, a subject is an individual to whom diagnosis and/or therapy is and/or has been administered (e.g., gene therapy for Wilson disease). In some embodiments, a subject is a human patient with Wilson disease.

As used herein, the term “substantial” or “substantially” refers to the qualitative condition of exhibition of total or near-total extent or degree of a characteristic or property of interest. One of ordinary skill in the art will understand that biological and chemical phenomena rarely, if ever, go to completion and/or proceed to completeness or achieve or an absolute result. The term “substantial” or “substantially” is therefore used herein to capture the potential lack of completeness inherent in many biological and chemical phenomena.

As used herein, the term “therapeutic polypeptide” is a peptide, polypeptide or protein (e.g., enzyme, structural protein, transmembrane protein, transport protein) that may alleviate or reduce symptoms that result from an absence or defect in a protein in a target cell (e.g., an isolated cell) or organism (e.g., a subject). A therapeutic polypeptide or protein encoded by a transgene is one that confers a benefit to a subject, e.g., to correct a genetic defect, to correct a deficiency in a gene related to expression or function. Similarly, a “therapeutic transgene” is the transgene that encodes the therapeutic polypeptide. In some embodiments, a therapeutic polypeptide, expressed in a host cell, is an enzyme expressed from a transgene (i.e., an exogenous nucleic acid that has been introduced into the host cell). In some embodiments, a therapeutic polypeptide is a copper transporting ATPase 2 protein, or fragment thereof, expressed from a therapeutic transgene transduced into a liver cell.

As used herein, the term “transgene” is used to mean any heterologous polynucleotide for delivery to and/or expression in a host cell, target cell or organism (e.g., a subject). Such “transgene” may be delivered to a host cell, target cell or organism using a vector (e.g., rAAV vector). A transgene may be operably linked to a control sequence, such as a promoter. It will be appreciated by those of skill in the art that expression control sequences can be selected based on an ability to promote expression of the transgene in a host cell, target cell or organism. Generally, a transgene may be operably linked to an endogenous promoter associated with the transgene in nature, but more typically, the transgene is operably linked to a promoter with which the transgene is not associated in nature. An example of a transgene is a nucleic acid encoding a therapeutic polypeptide, for example a copper-transporting ATPase 2 polypeptide, or fragment thereof, and an exemplary promoter is one not operable linked to a nucleotide encoding copper transporting ATPase 2 polypeptide in nature. Such a non-endogenous promoter can include an α1-antitrypsin (AAT) promoter, a portion of an AAT promoter, or a liver specific promoter, among many others known in the art. In some embodiments, a portion of an AAT promoter is a minimal AAT promoter as disclosed in WO 2016/097218 and WO 2016/097219, both of which are incorporated herein by reference.

As used herein, the term “treatment” refers to an approach for obtaining beneficial or desired clinical results. For purposes of this disclosure, beneficial or desired clinical results include, but are not limited to, one or more of the following: restoration of cooper metabolism (e.g., as measured by increased fecal and renal excretion of cooper, decreased serum cooper and decreased hepatic copper), increased serum ceruloplasmin levels, decreased urinary copper, decreased hepatic copper, reversal of liver injury (e.g., normalization of hepatic function and enzyme parameters), reduced or no need for treatment with zinc salts and/or chelation therapy, serum alanine transaminase levels less than/equal to the upper limit of normal, serum aspartate transaminase levels less than/equal to the upper limit of normal, and decreased neurological symptoms.

As used herein, the term “vector” refers to a plasmid, virus (e.g., a rAAV), cosmid, or other vehicle that can be manipulated by insertion or incorporation of a nucleic acid (e.g., a recombinant nucleic acid). A vector can be used for various purposes including, e.g., genetic manipulation (e.g., cloning vector), to introduce/transfer a nucleic acid into a cell, to transcribe or translate an inserted nucleic acid in a cell. In some embodiments, a vector nucleic acid sequence contains at least an origin of replication for propagation in a cell. In some embodiments, a vector nucleic acid includes a heterologous nucleic acid sequence, an expression control element(s) (e.g., promoter, enhancer), a selectable marker (e.g., antibiotic resistance), a poly-adenosine (polyA) signal sequence and/or an ITR. In some embodiments, when delivered to a host cell, the nucleic acid sequence is propagated. In some embodiments, when delivered to a host cell, either in vitro or in vivo, the cell expresses the polypeptide encoded by the heterologous nucleic acid sequence (e.g., a transgene). In some embodiments, when delivered to a host cell, the nucleic acid sequence, or a portion of the nucleic acid sequence is packaged into a capsid. A host cell may be an isolated cell or a cell within a host organism. In addition to a nucleic acid sequence (e.g., transgene) which encodes a polypeptide or protein, additional sequences (e.g., regulatory sequences) may be present within the same vector (i.e., in cis to the gene) and flank the gene. In some embodiments, regulatory sequences may be present on a separate (e.g., a second) vector which acts in trans to regulate the expression of the gene. Plasmid vectors may be referred to herein as “expression vectors.”

As used herein, the term “vector genome” refers to a nucleic acid that that may, but need not, be packaged/encapsidated in an AAV capsid to form a rAAV vector. Typically, a vector genome includes a heterologous polynucleotide sequence (e.g., a transgene, regulatory elements, etc.) and at least one ITR. In cases where a recombinant plasmid is used to construct or manufacture a recombinant vector (e.g., rAAV vector), the vector genome does not include the entire plasmid but rather only the sequence intended for delivery by the viral vector. This non-vector genome portion of the recombinant plasmid is referred to as the “plasmid backbone,” which is important for cloning, selection and amplification of the plasmid, a process that is needed for propagation of recombinant viral vector production, but which is not itself packaged or encapsidated into a rAAV vector. Typically, the heterologous sequence to be packaged into the capsid is flanked by the ITRs such that when cleaved from the plasmid backbone, the heterologous sequence is packaged into the capsid.

As used herein, the term “viral particle/mL” or “vp/mL” refers the number, amount or level of intact AAV capsids in a solution, such as a pharmaceutical formulation. Intact AAV capsids include full capsids, empty capsids and intermediate capsids. Vp/mL may be expressed as a titer when serving as a quality attribute for a pharmaceutical formulation.

As used herein, the term “viral vector” generally refers to a viral particle that functions as a nucleic acid delivery vehicle and which comprises a vector genome (e.g., comprising a transgene which has replaced the wild type rep and cap) packaged within the viral particle (i.e., capsid) and includes, for example, lenti- and parvo-viruses, including AAV serotypes and variants (e.g., rAAV vectors). As noted elsewhere herein, a recombinant viral vector does not comprise a virus genome with a rep and/or a cap gene; rather, these sequences have been removed to provide capacity for the vector genome to carry a transgene of interest.

As used herein, the term, “viscosity,” may be “absolute viscosity” or “kinematic viscosity.” “Absolute viscosity,” sometimes called dynamic or simple viscosity, is a quantity that describes a fluid's resistance to flow. “Kinematic viscosity” is the quotient of absolute viscosity and fluid density. Kinematic viscosity is frequently reported when characterizing the resistive flow of a fluid using a capillary viscometer. When two fluids of equal volume are placed in identical capillary viscometers and allowed to flow by gravity, a viscous fluid takes longer than a less viscous fluid to flow through the capillary. If one fluid takes 200 seconds to complete its flow and another fluid takes 400 seconds, the second fluid is twice as viscous as the first on a kinematic viscosity scale. If both fluids have equal density, the second fluid is twice as viscous as the first on an absolute viscosity scale. The dimensions of kinematic viscosity are L²/T where L represents length and T represents time. The SI units of kinematic viscosity are m²/s. Commonly, kinematic viscosity is expressed in centistokes, cSt, which is equivalent to mm²/s. The dimensions of absolute viscosity are M/L/T, where M represents mass and L and T represent length and time, respectively. The SI units of absolute viscosity are Pa·s, which is equivalent to kg/m/s. The absolute viscosity is commonly expressed in units of centiPoise, cP, which is equivalent to milliPascal-second, mPa·s.

Unless otherwise defined, all technical and scientific terms used herein have the same meaning as commonly understood by one of ordinary skill in the art to which this invention belongs.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

The present disclosure provides pharmaceutical compositions comprising rAAV vectors and excipients. In particular, the disclosure provides pharmaceutical compositions comprising a rAAV vector, a buffer, a salt, a cryoprotectant and a surfactant which is stable during extended storage (e.g., 6 months) and under stress conditions (e.g., sheer and freeze/thaw). Each of these aspects of the disclosure is discussed further in the ensuing sections.

### II. General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### III. AAV and rAAV

### A. AAV

“Adeno-associated virus” and/or “AAV” refer to parvoviruses with a linear single-stranded DNA genome and variants thereof. The term covers all subtypes and both naturally occurring and recombinant forms, except where required otherwise. Parvoviruses, including AAV, are useful as gene therapy vectors as they can penetrate a cell and introduce a nucleic acid (e.g., transgene) into the nucleus. In some embodiments, the introduced nucleic acid (e.g., rAAV vector genome) forms circular concatemers that persist as episomes in the nucleus of transduced cells. In some embodiments, a transgene is inserted in specific sites in the host cell genome, for example at a site on human chromosome 19. Site-specific integration, as opposed to random integration, is believed to likely result in a predictable long-term expression profile. The insertion site of AAV into the human genome is referred to as AAVS1. Once introduced into a cell, polypeptides encoded by the nucleic acid can be expressed by the cell. Because AAV is not associated with any pathogenic disease in humans, a nucleic acid delivered by AAV can be used to express a therapeutic polypeptide for the treatment of a disease, disorder and/or condition in a human subject.

The canonical AAV wild-type genome comprises 4681 bases (Berns et al. (1987) Advances in Virus Research 32:243-307) and includes terminal repeat sequences (e.g., inverted terminal repeats (ITRs)) at each end which function in cis as origins of DNA replication and as packaging signals for the virus. The genome includes two large open reading frames, known as AAV replication (“AAV rep” or “rep”) and capsid (“AAV cap” or “cap”) genes, respectively. AAV rep and cap may also be referred to herein as AAV “packaging genes.” These genes code for the viral proteins involved in replication and packaging of the viral genome.

Wild type AAV comprises a small (20-25 nm) icosahedral virus capsid composed of three proteins, VP1, VP2 and VP3, with 60 capsid proteins comprising the capsid. The three capsid genes VP1, VP2 and VP3 overlap each other within a single open reading frame and alternative splicing leads to production of VP1, VP2 and VP3 (Grieger et al. (2005) J. Virol. 79(15):9933-9944.). A single P40 promoter allows all three capsid proteins to be expressed at a ratio of about 1:1:10 for VP1, VP2, VP3, respectively, which complements AAV capsid production. More specifically, VP1 is the full-length protein, with VP2 and VP3 being increasingly shortened due to increasing truncation of the N-terminus. A well-known example is the capsid of AAV9 as described in U.S. Pat. No. 7,906,111, wherein VP1 comprises amino acid residues 1 to 736 of a sequence identified as number 123, VP2 comprises amino acid residues 138 to 736 of a sequence identified as number 123, and VP3 comprises amino acid residues 203 to 736 of a sequence identified as number 123. The AAV2 capsid protein sequences are available in Genbank: VP1 (735 aa; Genbank Accession No. AAC03780), VP2 (598 aa; Genbank Accession No. AAC03778) and VP3 (533 aa; Genbank Accession No. AAC03779). As used herein, the term “AAV Cap” or “cap” refers to AAV capsid proteins VP1, VP2 and/or VP3, and variants and analogs thereof.

A second open reading frame of the capsid gene encodes an assembly factor, called assembly-activating protein (AAP), which is essential for the capsid assembly process (Sonntag et al. (2011) J. Virol. 85(23):12686-12697).

At least four viral proteins are synthesized from the AAV rep gene-Rep 78, Rep 68, Rep 52 and Rep 40—named according to their apparent molecular weights. As used herein, “AAV rep” or “rep” means AAV replication proteins Rep 78, Rep 68, Rep 52 and/or Rep 40, as well as variants and analogs thereof. As used herein, rep and cap refer to both wild type and recombinant (e.g., modified chimeric, and the like) rep and cap genes as well as the polypeptides they encode. In some embodiments, a nucleic acid encoding a rep will comprise nucleotides from more than one AAV serotype. For instance, a nucleic acid encoding a rep protein may comprise nucleotides from an AAV2 serotype and nucleotides from an AAV3 serotype (Rabinowitz et al. (2002) J. Virology 76(2):791-801).

Multiple serotypes of AAV exist in nature with at least fifteen wild type serotypes having been identified from humans thus far (i.e., AAV1-AAV15). Naturally occurring and variant serotypes are distinguished by having a protein capsid that is serologically distinct from other AAV serotypes. Naturally occurring and non-naturally occurring AAV serotypes include: AAV type 1 (AAV1), AAV type 2 (AAV2), AAV type 3 (AAV3) including AAV type 3A (AAV3A) and AAV type 3B (AAV3B), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 12 (AAV12), AAVrh10, AAVrh74 (see WO 2016/210170), AAV1.1, AAV2.5, AAV6.1, AAV6.3.1, AAV9.45, RHM4-1 (SEQ ID NO:5 of WO 2015/013313), RHM15-1, RHM15-2, RHM15-3/RHM15-5, RHM15-4, RHM15-6, AAV hu.26, AAV2i8, AAV29G, AAV2,8G9, AAV-LK03, AAV2-TT, AAV2-TT-S312N, AAV3B-S312N, avian AAV, bovine AAV, canine AAV, equine AAV, primate AAV, non-primate AAV, and ovine AAV, AAV type 2i8 (AAV2i8), NP4, NP22, NP66, AAVDJ, AAVDJ/8, AAVDJ/9, among many others (see, e.g., Fields et al., “Virology”, volume 2, chapter 69 (4^{th} ed., Lippincott-Raven Publishers); U.S. Pat. No. 7,906,111; Gao et al. (2004) J. Virol. 78:6381; Morris et al. (2004) Virol. 33:375; WO 2013/063379; WO 2014/194132; WO 2015/121501; WO 2015/013313, all of which are hereby incorporated by reference). AAV variants isolated from human CD34+ cell include AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15 (Smith et al. (2014) Molecular Therapy 22(9): 1625-1634, which is hereby incorporated by reference).

Serotype distinctiveness is determined on the basis of the lack of cross-reactivity between antibodies to one AAV as compared to another AAV. Such cross-reactivity differences are usually due to differences in capsid protein sequences and antigenic determinants (e.g., due to VP1, VP2, and/or VP3 sequence differences of AAV serotypes). However, some naturally occurring AAV or man-made AAV mutants (e.g., recombinant AAV) may not exhibit serological difference with any of the currently known serotypes. These viruses may then be considered a subgroup of the corresponding type, or more simply a variant AAV. Thus, as used herein, the term “serotype” refers to both serologically distinct viruses, e.g., AAV, as well as viruses, e.g., AAV, that are not serologically distinct but that may be within a subgroup or a variant of a given serotype.

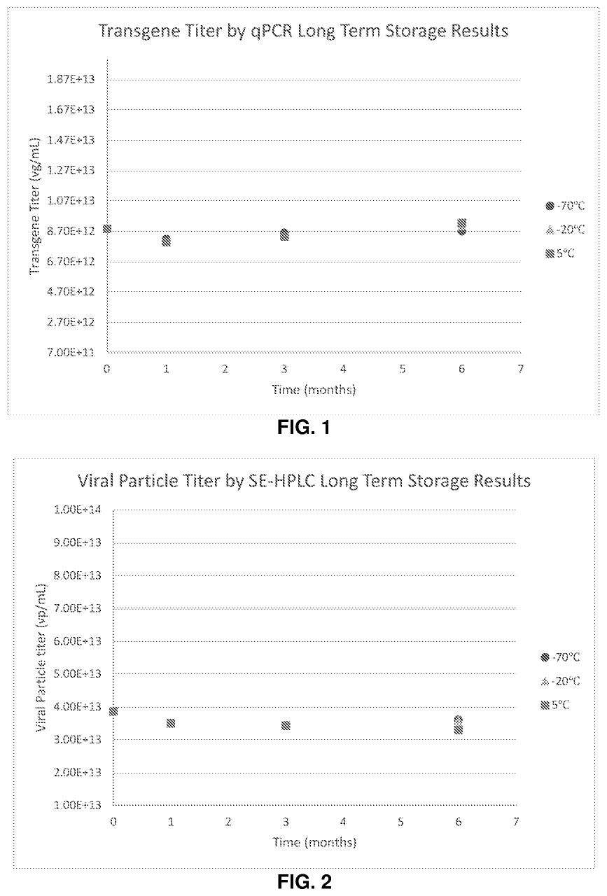

A comprehensive list and alignment of amino acid sequences of capsids of known AAV serotypes is provided by Marsic et al. (2014) Molecular Therapy 22(11):1900-1909, especially at supplementary FIG. 1; the entire publication is hereby incorporated by reference.

Genomic sequences of various serotypes of AAV, as well as sequences of the native inverted terminal repeats (ITRs), rep proteins, and capsid subunits are known in the art. Such sequences may be found in the literature or in public databases such as GenBank. See, e.g., GenBank Accession Numbers NC_002077 (AAV1), AF063497 (AAV1), NC_001401 (AAV2), AF043303 (AAV2), NC_001729 (AAV3), AF028705.1 (AAV3B), NC_001829 (AAV4), U89790 (AAV4), NC_006152 (AAV5), AF028704 (AAV6), AF513851 (AAV7), AF513852 (AAV8), NC_006261 (AAV8), AY530579 (AAV9), AY631965 (AAV10), AY631966 (AAV11), and DQ813647 (AAV12); the disclosures of which are incorporated by reference herein. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al. (1986) J. Virol. 58:921; Gao et al. (2002) Proc. Nat. Acad. Sci. USA 99: 11854; Moris et al. (2004) Virology 33:375-383; International Patent Publications WO 00/28061, WO 99/61601, WO 98/11244; WO 2013/063379; WO 2014/194132; WO 2015/121501, and U.S. Pat. Nos. 6,156,303 and 7,906,111, all of which are hereby incorporated by reference.

In one embodiment of a pharmaceutical composition disclosed herein, a rAAV vector comprises an AAV3B VP1 polypeptide (also referred to herein as a “capsid protein”) comprising the amino acid sequence of SEQ ID NO:10. AAV3B VP2 and VP3 encompass about amino acids 138 to 736 and about amino acids 203 to 736 of SEQ ID NO:10 (GenBank accession no. AAB95452.1). In one embodiment of a pharmaceutical composition disclosed herein, a rAAV vector comprises an AAV3B VP1 polypeptide (also referred to herein as a “capsid protein”) encoded by the nucleic acid sequence of SEQ ID NO:11 (nucleotides 2208-4418 of GenBank accession no. AF028705.1).

B. Recombinant AAV (rAAV)

A “recombinant adeno-associated virus,” or “rAAV” (also referred to herein as a “rAAV vector,” “rAAV viral particle,” and/or “rAAV vector particle”) refers to an AAV capsid comprising a vector genome, unless specifically noted otherwise. The vector genome comprises a polynucleotide sequence that is not, at least in part, derived from a naturally-occurring AAV (e.g., a heterologous polynucleotide not present in wild type AAV), and wherein the rep and/or cap genes of the wild type AAV genome have been removed from the vector genome. ITRs from an AAV have been added or remain in the vector genome. Therefore, the term rAAV vector encompasses a rAAV viral particle that comprises a capsid but does not comprise a complete AAV genome; instead the recombinant viral particle can comprise a heterologous, i.e., not originally present in the capsid, nucleic acid, the vector genome. Thus, a “rAAV vector genome” (or “vector genome”) refers to a heterologous polynucleotide sequence (including at least one ITR) that may, but need not, be contained within an AAV capsid. A rAAV vector genome may be double-stranded (dsAAV), single-stranded (ssAAV) or self-complementary (scAAV). Typically, a vector genome comprises a heterologous nucleic acid often encoding a therapeutic transgene, for example an ATP7B gene, or fragment thereof, as provided in SEQ ID NO:2. In some embodiments, a vector genome comprises a heterologous nucleic acid encoding a copper-transporting ATPase 2 protein, or fragment thereof, as provided in SEQ ID NO:1.

A rAAV vector, and those terms provided above, are to be distinguished from an “AAV viral particle” or “AAV virus” that is not recombinant, contains a virus genome encoding rep and cap genes, and which AAV virus is capable of replicating when present in a cell also comprising a helper virus, such as an adenovirus and/or herpes simplex virus, and/or required helper genes therefrom. Thus, production of a rAAV vector necessarily includes production of a recombinant vector genome using recombinant DNA technologies, and wherein the recombinant vector genome is contained within an AAV capsid to form the rAAV vector.

The present disclosure provides for a pharmaceutical composition comprising a rAAV vector, and methods of use thereof. In some embodiments, the rAAV vector comprises an AAV3B capsid and optionally, a transgene encoding a polypeptide that is a target for therapeutic treatment (e.g., a nucleic acid encoding a copper-transporting ATPase 2, or a fragment thereof, for the treatment of Wilson disease, e.g., SEQ ID NO:2). Delivery or administration of a rAAV vector to a subject (e.g. a patient) provides encoded proteins and peptides to the subject. Thus, a rAAV vector can be used to transfer/deliver a heterologous polynucleotide for expression for the treatment of diseases, disorders and/or conditions. In some embodiments, a rAAV vector transfers a copy of an ATP7B, or fragment thereof (e.g., an ATP7B with deletion of the MBS1-4 coding regions) to hepatocytes which is expressed as a shortened copper transporting ATPase 2 for the treatment of Wilson disease.

A rAAV vector genome generally retains 130 to 145 base ITRs in cis to the heterologous nucleic acid sequence that replaces the viral rep and cap genes. Such ITRs are necessary to produce a recombinant AAV vector as they mediate AAV genome replication and packaging. However, modified AAV ITRs and non-AAV terminal repeats including partially or completely synthetic sequences can also serve this purpose. ITRs form hairpin structures and function to, for example, serve as primers for host-cell-mediated synthesis of the complementary DNA strand after infection. ITRs also play a role in viral packaging, integration, etc. ITRs are the only AAV viral elements which are required in cis for AAV genome replication and packaging into rAAV vectors. A rAAV vector genome optionally comprises two ITRs which are generally at the 5′ and 3′ ends of the vector genome comprising a heterologous sequence (e.g., a transgene encoding a gene of interest). A 5′ and a 3′ ITR may both comprise the same sequence, or each may comprise a different sequence (e.g., SEQ ID NO:5, SEQ ID NO:6, SEQ ID NO:7, SEQ ID NO:8). In some embodiments, a rAAV vector genome of the disclosure comprises an ITR comprising or consisting of the nucleic acid sequence of any one of SEQ ID NO:5-8. In some embodiments, a rAAV vector genome of the disclosure comprises an ITR comprising a nucleic acid sequence that is, or is at least, or is at most 80%, 85%, 90%, 95%, 98%, 99% or 100% identical to any one of SEQ ID NO:5-8. An AAV ITR may be from any AAV, including but not limited to, serotypes 1, 2, 3, 4, 5, 6, 7, 8, 9, 10 or 11 or any other AAV.

A rAAV vector genome of the disclosure may comprise an ITR from an AAV serotype (e.g., wild-type AAV2, a fragment or variant thereof) that differs from the serotype of the capsid (e.g., AAV3B or other). Such a rAAV vector genome comprising at least one ITR from one serotype, but comprising a capsid from a different serotype, may be referred to as a hybrid viral vector (see U.S. Pat. No. 7,172,893). An rAAV ITR may include the entire wild type ITR sequence, or be a variant, fragment, or modification thereof, but will retain functionality.

In addition to a transgene and at least one ITR, a vector genome may also include various regulatory or control elements. Typically, regulatory elements are nucleic acid sequence(s) that influence expression of an operably linked polynucleotide (e.g., a transgene). The precise nature of regulatory elements useful for gene expression will vary from organism to organism and from cell type to cell type including, for example, a promoter, enhancer, intron etc., with the intent to facilitate proper heterologous polynucleotide transcription and translation. Regulatory control can be affected at the level of transcription, translation, splicing, message stability, etc.

In some embodiments, a pharmaceutical composition of the disclosure comprises an rAAV vector comprising a recombinant nucleic acid comprising at least one ITR, a transgene, a promoter and a polyadenylation signal (polyA) sequence. In some embodiments, a transgene encodes a copper-transporting ATPase 2, or a fragment thereof. In some embodiments, a transgene encodes a copper-transporting ATPase 2, or a fragment thereof comprising or consisting of the amino acid sequence of SEQ ID NO:1. In some embodiments, a transgene encodes a copper-transporting ATPase 2, or a fragment thereof comprising an amino acid that is, or is at least, or is at most 80%, 85%, 90%, 95%, 98%, 99% or 100% identical to SEQ ID NO:1.

In some embodiments, an ATP7B transgene is disclosed in WO2016/097219 (nucleotides 473-3580 of SEQ ID NO:6, incorporated herein by reference). In some embodiments, a transgene comprises or consists of the nucleic acid of SEQ ID NO:2 which encodes a copper-transporting ATPase 2, or a fragment thereof. In some embodiments, a transgene is an ATP7B gene, or fragment thereof (e.g., SEQ ID NO:2). In some embodiments, a transgene comprises a nucleic acid sequence that is, or is at least, or is at most 80%, 85%, 90%, 95%, 98%, 99% or 100% identical to the nucleic acid sequence of SEQ ID NO:2.

In some embodiments, a promoter is a minimal AAT promoter. In some embodiments, a minimal AAT promoter is disclosed in WO2016/097219 (nucleotides 156-460 of SEQ ID NO:1; incorporated herein by reference). In some embodiments, a promoter comprises or consists of the nucleic acid sequence of SEQ ID NO:3. In some embodiments, a promoter comprises a nucleic acid sequence that is, or is at least, or is at most 80%, 85%, 90%, 95%, 98%, 99% or 100% identical to the nucleic acid sequence of SEQ ID NO:3.

In some embodiments, a polyA signal sequence is from a rabbit β-globin gene. In some embodiments, a polyA signal sequence is disclosed in WO2016/097219 (nucleotides 4877-4932 of SEQ ID NO:1; incorporated herein by reference). In some embodiments, a polyA signal sequence comprises or consists of the nucleic acid sequence of SEQ ID NO:4. In some embodiments, a polyA signal sequence comprises a nucleic acid sequence that is, or is at least, or is at most 80%, 85%, 90%, 95%, 98%, 99% or 100% identical to the nucleic acid sequence of SEQ ID NO:4.

In some embodiments, an exemplary vector genome comprises a nucleic acid encoding a copper-transporting ATPase 2, a minimal AAT promoter, a polyA sequence and two ITR sequences. In some embodiments, a vector genome comprises: a nucleic acid encoding a copper-transporting ATPase 2 with a deletion of metal binding sites (MBS) 1-4 and/or encoding a copper transporting ATPase 2 comprising or consisting of the amino acid sequence of SEQ ID NO:1, a minimal AAT promoter comprising the nucleic acid sequence of SEQ ID NO:3, a polyA comprising the nucleic acid sequence of SEQ ID NO:4 and two AAV2 ITR sequences. In some embodiments, the ITR sequences comprise the nucleic acid sequence of any one of SEQ ID NO:5-8.

A viral capsid of a rAAV vector may be, but not limited to, any of the wild type AAV and variant AAV, described above. In some embodiments, a viral capsid polypeptide is of an AAV serotype selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1, RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15.

In some embodiments, a viral capsid of a rAAV vector is an AAV3B capsid. In some embodiments, a viral capsid of a rAAV vector comprises a polypeptide encoded by at least a portion of the sequence of GenBank accession no. AF028705.1 (e.g., nucleotides 2208-4418). In some embodiments, a viral capsid of a rAAV vector comprises a polypeptide encoded by a nucleic acid sequence comprising or consisting of SEQ ID NO:11. In some embodiments, a viral capsid of a rAAV vector comprises a polypeptide encoded by a nucleic acid sequence that is, or is at least, or is at most 80%, 85%, 90%, 95%, 98%, 99% or 100% identical to the nucleic acid sequence of SEQ ID NO:10.

In some embodiments, a viral capsid of a rAAV vector comprising a polypeptide comprising or consisting of the amino acid sequence of GenBank accession no. AAB95452.1. In some embodiments, a viral capsid of a rAAV vector comprises a polypeptide comprising or consisting of the amino acid sequence of SEQ ID NO:10. In some embodiments, a viral capsid of a rAAV vector is a polypeptide that is, or is at least, or is at most 80%, 85%, 90%, 95%, 98%, 99% or 100% identical to the amino acid sequence of SEQ ID NO:10.

In some embodiments, a rAAV vector comprises i) a vector genome comprising: a nucleic acid encoding a copper-transporting ATPase 2 with a deletion of metal binding sites (MBS) 1-4 and/or encoding a copper transporting ATPase 2 comprising the amino acid sequence of SEQ ID NO:1, a minimal AAT promoter comprising the nucleic acid sequence of SEQ ID NO:3, a polyA comprising the nucleic acid sequence of SEQ ID NO:4 and two AAV2 ITR sequences, optionally comprising the nucleic acid sequence of any one of SEQ ID NO:5-8 and ii) a capsid comprising an AAV3B capsid (e.g., the amino acid sequence of SEQ ID NO:10).

In some embodiments, the present disclosure provides for the use of ancestral AAV vectors for use in rAAV vectors for in vivo gene therapy. Specifically, in silico-derived sequences may be synthesized de novo and characterized for biological activities. Prediction and synthesis of ancestral sequences, in addition to assembly into a rAAV vector, may be accomplished using methods described in WO 2015/054653, the contents of which are incorporated by reference herein. Notably, rAAV vectors assembled from ancestral viral sequences may exhibit reduced susceptibility to pre-existing immunity in human populations as compared to contemporary viruses or portions thereof.

In some embodiments, a rAAV vector comprising a capsid protein encoded by a nucleotide sequence derived from more than one AAV serotype (e.g., wild type AAV serotypes, variant AAV serotypes) is referred to as a “chimeric vector” or “chimeric capsid” (See U.S. Pat. No. 6,491,907, the entire disclosure of which is incorporated herein by reference). In some embodiments, a chimeric capsid protein is encoded by a nucleic acid sequence derived from 2, 3, 4, 5, 6, 7, 8, 9, 10 or more AAV serotypes. In some embodiments, a recombinant AAV vector includes a capsid sequence derived from e.g., AAV1, AAV2, AAV3, AAV3A, AAV3B, AAV4, AAV5, AAV6, AAV7, AAV8, AAV9, AAV10, AAV11, AAVrh74, AAVrh10, AAV2i8, or variant thereof, resulting in a chimeric capsid protein comprising a combination of amino acids from any of the foregoing AAV serotypes (see, Rabinowitz et al. (2002) J. Virology 76(2):791-801). Alternatively, a chimeric capsid can comprise a mixture of a VP1 from one serotype, a VP2 from a different serotype, a VP3 from yet a different serotype, and a combination thereof. For example, a chimeric virus capsid may include an AAV1 cap protein or subunit and at least one AAV2 cap protein or subunit. A chimeric capsid can, for example include an AAV capsid with one or more B19 cap subunits, e.g., an AAV cap protein or subunit can be replaced by a B19 cap protein or subunit. For example, in one embodiment, a VP3 subunit of an AAV capsid can be replaced by a VP2 subunit of B19.

In some embodiments, chimeric vectors have been engineered to exhibit altered tropism or tropism for a particular tissue or cell type. The term “tropism” refers to preferential entry of the virus into certain cell or tissue types and/or preferential interaction with the cell surface that facilitates entry into certain cell or tissue types. AAV tropism is generally determined by the specific interaction between distinct viral capsid proteins and their cognate cellular receptors (Lykken et al. (2018) J. Neurodev. Disord. 10:16). Preferably, once a virus or viral vector has entered a cell, sequences (e.g., heterologous sequences such as a transgene) carried by the vector genome (e.g., a rAAV vector genome) are expressed.

A “tropism profile” refers to a pattern of transduction of one or more target cells, tissues and/or organs. For example, an AAV capsid may have a tropism profile characterized by efficient transduction of muscle cells with only low transduction of, for example, brain cells.

The rAAV vectors described herein may be obtained by any known production systems, such as mammalian cell AAV production systems (e.g., those based on 293T or HEK293 cells) and insect cell AAV production systems (e.g., those based on sf9 insect cells and/or those using baculoviral helper vectors).

A rAAV vector may be purified by methods standard in the art such as by any number of column chromatography methods (e.g., affinity chromatography, ion exchange chromatography, hydrophobic interaction chromatography) or cesium chloride gradients. Methods for purifying rAAV vectors are known in the art and include methods described in Clark et al. (1999) Human Gene Therapy 10(6):1031-1039; Schenpp et al. (2002) Methods Mol. Med. 69:427-443; U.S. Pat. No. 6,566,118 and WO 98/09657.

After rAAV vectors have been produced and purified, they can be titered (e.g., the amount of rAAV vector in a sample can be quantified) to prepare compositions for administration to subjects, such as human subjects with Wilson disease. rAAV vector titering can be accomplished using methods know in the art.

### C. Exemplary Recombinant AAV

In exemplary embodiments, the present disclosure provides an improved pharmaceutical composition comprising a rAAV vector for treatment of Wilson Disease (WD) to, for example, the restoration of normal biliary and/or fecal excretion of cooper and normalization of loading of cooper into ceruolplasmin. The rAAV vector comprises a AAV3B capsid and a vector genome with AAV2 ITRs flanking an AAT promoter, a ATP7B transgene, with deletion of MBS1-4, and a polyA signal sequence (see, e.g., WO2016/097219, and WO2016/097218, each of which are incorporated herein by reference).

In some embodiments, the rAAV vector comprises i) a vector genome comprising: a nucleic acid encoding a copper-transporting ATPase 2 with a deletion of metal binding sites (MBS) 1-4 and/or encoding a copper transporting ATPase 2 comprising the amino acid sequence of SEQ ID NO:1, a minimal AAT promoter comprising the nucleic acid sequence of SEQ ID NO:3, a polyA comprising the nucleic acid sequence of SEQ ID NO:4 and two AAV2 ITR sequences, optionally comprising the nucleic acid sequence of any one of SEQ ID NO:5-8 and ii) a capsid comprising an AAV3B capsid. In some embodiments, the AAV3B capsid polypeptide comprises the amino acid sequence set forth in SEQ ID NO:10 and at GenBank accession no. AAB95452.1 and/or encoded by the nucleotide sequence set forth in SEQ ID NO:11 and at nucleotides 2208-4418 of GenBank accession no. AF028705.1.

### IV. Formulation of rAAV Vectors

Once purified, AAV preparations can be formulated as described herein, for example, by buffer exchange through tangential flow filtration, normal flow filtration using stir-cells, gel filtration, dialysis, column chromatography, and/or desalting columns, to arrive at a composition comprising the desired ingredients. By way of illustration, the purified viral preparation may be concentrated first by ultrafiltration (UF) and then diafiltrated (DF).

A formulation may comprise a buffering agent, a salt which provides a divalent cation, a cryprotectant and a surfactant. As used herein “buffer” refers to an added composition that allows a liquid rAAV vector formulation to resist changes in pH, typically by action of its acid-base conjugate components. When a concentration of a buffer is referred to, it is intended that the recited concentration represent the molar concentration of the free acid or free base form of the buffer. The pI for AAVs is approximately 6.3, thus a higher pH is optimal for a formulation comprising a rAAV vector. A downward shift in pH of a formulation, comprising a rAAV vector, and in particular a rAAV3B vector, can cause conformational changes in the vector, thereby reducing its stability as shown by changes in quality attribute parameters. The pH of some buffers, such as phosphate buffered saline (PBS) are known in the art to shift downward upon freezing. The pH of other buffers do not exhibit such shifts when frozen, making such buffers (e.g., Tris) preferable for formulations comprising a rAAV vector that will be frozen during storage.

Buffering agents may include, for example, acetate, succinate (e.g., disodium succinate hexahydrate), succinic acid, gluconate, citrate, histidine, acetic acid, phosphate, phosphoric acid, ascorbate, ascorbic acid, tartaric acid, malate, maleic acid, glycine, lactate, lactic acid, bicarbonate, carbonic acid, sodium benzoate, benzoic acid, edetate, imidazole, Tris (e.g., Tris base, Tris HCL or both), and mixtures thereof. In some embodiments, a composition comprising a rAAV vector comprises Tris.

In some embodiments, the concentration of a buffer (e.g., Tris) in a formulation comprising a rAAV vector is about 1 mM to about 500 mM Tris, e.g., about 1 mM to about 450 mM, about 1 mM to about 400 mM, about 1 mM to about 350 mM, about 1 mM to about 300 mM, about 1 mM to about 250 mM, about 1 mM to about 200 mM, about 1 mM to about 150 mM, about 1 mM to about 100 mM, about 1 mM to 75 mM, about 1 mM to 50 mM, about 1 mM to 25 mM, about 5 mM to about 30 mM, about 10 mM to about 30 mM or about 14 mM to about 26 mM.

In some embodiments, a concentration of a buffer (e.g., Tris) in a formulation comprising a rAAV vector is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM or about 500 mM. In some embodiments, a concentration of Tris in a formulation comprising a rAAV (e.g., AAV3B) vector is about 20 mM.

As used herein, the term “divalent cation” refers to a cation with a valence of +2. This type of ion may form two chemical bonds with an anion. Divalent cations may be provided in a formulation as a salt, including, for example, MgCl₂, MgSO₄, and CaCl₂. A divalent cation ion in a formulation comprising a rAAV vector, and in particular a rAAV3B vector, allows the formulation to have a high ionic strength without being significantly hypertonic. Without wishing to be bound by any particular theory, a salt, such as MgCl₂ in a formulation stabilizes the positive charge patch on av AAV capsid, and in particular the VP3 charge patch of AAV3B capsids. In some embodiments, a monovalent cation may be provided in a formulation as a salt, including, for example, NaCl, Na₂SO₄ or sodium acetate.

In some embodiments, a concentration of a salt (e.g., MgCl₂) providing a divalent cation in a formulation comprising a rAAV vector is about 1 mM to about 500 mM, e.g., about 1 mM to about 450 mM, about 1 mM to about 400 mM, about 1 mM to about 350 mM, about 1 mM to about 300 mM, about 1 mM to about 250 mM, about 1 mM to about 200 mM, about 1 mM to about 150 mM, about 1 mM to about 100 mM, about 10 mM to about 450 mM, about 10 mM to about 400 mM, about 10 mM to about 350 mM, about 10 mM to about 300 mM, about 10 mM to about 250 mM, about 10 mM to about 200 mM, about 10 mM to about 175 mM, about 10 mM to about 150 mM, about 10 mM to about 125 mM, about 50 mM to about 450 mM, about 50 mM to about 400 mM, about 50 mM to about 350 mM, about 50 mM to about 300 mM, about 50 mM to about 250 mM, about 50 mM to about 200 mM, about 50 mM to about 150 mM or about 75 mM to about 450 mM, about 75 mM to about 400 mM, about 75 mM to about 350 mM, about 75 mM to about 300 mM, about 75 mM to about 250 mM, about 75 mM to about 200 mM, about 75 mM to about 150 mM, about 75 mM to about 125 mM or about 70 mM to about 130 mM.

In some embodiments, a concentration of a salt (e.g., MgCl₂) in a formulation is about 10 mM, about 20 mM, about 30 mM, about 40 mM, about 50 mM, about 60 mM, about 70 mM, about 80 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 120 mM, about 130 mM, about 140 mM, about 150 mM, about 160 mM, about 170 mM, about 180 mM, about 190 mM, about 200 mM, about 225 mM, about 250 mM, about 275 mM, about 300 mM, about 325 mM, about 350 mM, about 375 mM, about 400 mM, about 425 mM, about 450 mM, about 475 mM or about 500 mM. In some embodiments, a concentration of MgCl₂ in a formulation comprising a rAAV (e.g., rAAV3B) vector is about 100 mM.

As used herein, the term “cryoprotectant” refers to a molecule which, when combined with a protein (e.g., a rAAV vector), significantly prevents or reduces physiochemical instability and degradation of the protein upon freezing and subsequent storage. In some embodiments, a cryoprotectant functions to prevent the formation of ice crystals upon freezing of a liquid composition. A cryoprotectant in a formulation may also function as a tonicity modifier or a lyoprotectant. In some embodiments, a cryoprotectant may reduce the glass transition temperature (Tg′) of a rAAV vector such that it may be stored at about −40° C. as opposed to at about −70° C. In some embodiments, the glass transitions temperature (Tg′) of a rAAV vector is measured by modulated differential scanning calorimetry (mDSC). A cryoprotectant may be, for example, sugar (e.g., dextrose, lactose, glucose, fructose, maltose, mannose, sorbose, sucrose, trehalose, xylose), sugar alcohol (e.g., erythritol, ethylene glycol, glycerol, isomalt, inositol, lactitol, maltitol, mannitol, sorbitol, xylitol), amino acid (e.g., glycine, histidine, arginine), polypeptide, protein (e.g., albumin, gelatine), polymer (e.g., dextran, polyvinyl pyrrolidone, polyvinyl alcohol, propylene glycol, polyethylene glycol), water-soluble glucan, or any combination of such molecules.

In some embodiments, a formulation comprising a rAAV vector comprises about 0.1% to about 20% (w/v), e.g., about 0.1% to about 19% (w/v), about 0.1% to about 18% (w/v), about 0.1% to about 17% (w/v), about 0.1% to about 16% (w/v), about 0.1% to about 15% (w/v), about 0.1% to about 14% (w/v), about 0.1% to about 13% (w/v), about 0.1% to about 12% (w/v), about 0.1% to about 11% (w/v), about 0.1% to about 10% (w/v), about 0.1% to about 9% (w/v), about 0.1% to about 8% (w/v), about 0.1% to about 7% (w/v), about 0.1% to about 6% (w/v), about 0.1% to about 5% (w/v), about 0.1% to about 4% (w/v), about 0.1% to about 3% (w/v), about 0.1% to about 2% (w/v), about 0.1% to about 1% (w/v), about 1% to about 19% (w/v), about 1% to about 18% (w/v), about 1% to about 17% (w/v), about 1% to about 16% (w/v), about 1% to about 15% (w/v), about 1% to about 14% (w/v), about 1% to about 13% (w/v), about 1% to about 12% (w/v), about 1% to about 11% (w/v), about 1% to about 10% (w/v), about 1% to about 8% (w/v), about 1% to about 6% (w/v), about 2% to about 6% (w/v) or about 2.8% to about 5.2% (w/v) of a cryoprotectant (e.g., sucrose).

In some embodiments, a formulation comprising a rAAV vector comprises about 1% (w/v), about 2% (w/v), about 2.5% (w/v), about 2.6% (w/v), about 2.7% (w/v), about 2.8% (w/v), about 2.9% (w/v), about 3% (w/v), about 3.1% (w/v), about 3.2% (w/v), about 3.3% (w/v), about 3.4% (w/v), about 3.5% (w/v), about 3.6% (w/v), about 3.7% (w/v), about 3.8% (w/v), about 3.9% (w/v), about 4% (w/v), about 4.1% (w/v), about 4.2% (w/v), about 4.3% (w/v), about 4.4% (w/v), about 4.6% (w/v), about 4.7% (w/v), about 4.8% (w/v), about 4.9% (w/v), about 5% (w/v), about 5.1% (w/v), about 5.2% (w/v), about 5.3% (w/v), about 5.4% (w/v), about 5.5% (w/v), about 6% (w/v), about 7% (w/v), about 8% (w/v), about 9% (w/v), about 10% (w/v), about 11% (w/v), about 12% (w/v), about 13% (w/v), about 14% (w/v), about 15% (w/v), about 16% (w/v), about 17% (w/v), about 18% (w/v), about 19% (w/v) or about 20% (w/v) of a cryoprotectant (e.g., sucrose). In some embodiments, a formulation comprising a rAAV (e.g., rAAV3B) vector comprises about 4% (w/v) of sucrose.

As used herein, the term “surfactant” refers to an excipient that can alter the surface tension of a liquid rAAV vector formulation. A surfactant may provide one or more functions when formulated with a rAAV vector including protecting the vector from shear stress during manufacturing. In some embodiments, a surfactant reduces the surface tension of a liquid rAAV vector formulation. In still other embodiments, a “surfactant” may contribute to an improvement in stability of the rAAV vector in a formulation. A surfactant may reduce aggregation of a formulated rAAV vector and/or minimize formation of particulates in the formulation and/or reduce adsorption. A surfactant may also improve stability of a rAAV vector during and after a freeze/thaw cycle.

A surfactant may be, for example, a polysorbate, poloxamer (including poloxamer 188), triton, sodium dodecyl sulfate (SDS), sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, or any combination of such molecules. A surfactant may be, for example, polysorbate 20, polysorbate 21, polysorbate 40, polysorbate 60, polysorbate 61, polysorbate 65, polysorbate 80, polysorbate 81, polysorbate 85, PEG3350 or any combination of such molecules. In some embodiments, a surfactant is poloxamer 188.

In some embodiments, a formulation comprising a rAAV vector comprises about 0.001% (w/v) to about 0.5% (w/v), e.g., about 0.001% to about 0.4% (w/v), about 0.001% to about 0.3% (w/v), about 0.001% to about 0.2% (w/v), about 0.001% to about 0.1% (w/v), about 0.002% to about 0.5% (w/v), about 0.002% to about 0.4% (w/v), about 0.002% to about 0.3% (w/v), about 0.002% to about 0.2% (w/v), about 0.002% to about 0.15% (w/v), about 0.002% to about 0.1% (w/v), about 0.002% to about 0.05% (w/v), about 0.002% to about 0.04% (w/v), about 0.002% to about 0.03% (w/v), about 0.005% to about 0.5% (w/v), about 0.005% to about 0.4% (w/v), about 0.005% to about 0.3% (w/v), about 0.005% to about 0.2% (w/v), about 0.005% to about 0.15% (w/v), about 0.005% to about 0.1% (w/v), about 0.005% to about 0.05% (w/v), about 0.005% to about 0.04% (w/v), about 0.005% to about 0.03% (w/v), about 0.01% to about 0.5% (w/v), about 0.01% to about 0.4% (w/v), about 0.01% to about 0.3% (w/v), about 0.01% to about 0.2% (w/v), about 0.01% to about 0.1% (w/v), about 0.01% to about 0.05% (w/v), about 0.01% to about 0.03% (w/v) or about 0.014% to about 0.03% (w/v) of a surfactant (e.g., poloxamer 188).

In some embodiments, a formulation comprises about 0.005% (w/v), about 0.006% (w/v), about 0.007% (w/v), about 0.008% (w/v), about 0.009% (w/v), about 0.01% (w/v), about 0.014%, about 0.015% (w/v), 0.016% (w/v), 0.017% (w/v), about 0.018% (w/v), about 0.019% (w/v), about 0.02% (w/v), about 0.021% (w/v), about 0.022% (w/v), 0.023% (w/v), 0.024% (w/v), about 0.025% (w/v), about 0.026%, about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.8% (w/v), about 0.9% (w/v) or about 1% (w/v) of a surfactant (e.g., poloxamer 188). In some embodiments, a formulation comprising a rAAV vector comprises about 0.02% (w/v) poloxamer 188.

As used herein the term “lyoprotectant” refers to a molecule which, when combined with a protein of interest, significantly prevents or reduces physicochemical instability of the protein upon lyophilization and subsequent storage. Exemplary lyoprotectants include sugars and their corresponding sugar alcohols; an amino acid such as monosodium glutamate or histidine; a methylamine such as betaine; a lyotropic salt such as magnesium sulfate; a polyol such as trihydric or higher molecular weight sugar alcohols, e.g., glycerin, dextran, erythritol, glycerol, arabitol, xylitol, sorbitol, and mannitol; propylene glycol; polyethylene glycol; Pluronics®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred lyoprotectant are the non-reducing sugars trehalose or sucrose.

The lyoprotectant is added to the pre-lyophilized formulation in a “lyoprotecting amount” which means that, following lyophilization of the protein in the presence of the lyoprotecting amount of the lyoprotectant, the protein essentially retains its physicochemical stability upon lyophilization and storage.

In some embodiments, a formulation comprising a rAAV vector comprises about 0.1% (w/v) to about 10% (w/v), e.g., about 0.1% to about 9% (w/v), about 0.1% to about 8% (w/v), about 0.1% to about 7% (w/v), about 0.1% to about 6% (w/v), about 0.1% to about 5% (w/v), about 0.1% to about 4% (w/v), about 0.1% to about 3% (w/v), about 0.1% to about 2% (w/v), about 0.1% to about 1% (w/v), about 0.5% to about 10% (w/v), about 0.5% to about 9% (w/v), about 0.5% to about 8% (w/v), about 0.5% to about 7% (w/v), about 0.5% to about 6% (w/v), about 0.5% to about 5% (w/v), about 0.5% to about 4% (w/v), about 0.5% to about 3% (w/v), about 0.5% to about 2.5% (w/v), about 0.7% (w/v) to about 2.6% or about 1% to 2% of a lyoprotectant (e.g., sorbitol).

In some embodiments of the present disclosure, the pH of a formulation comprising a rAAV vector may be in the range of about 7 to about 8.5, e.g., about 7.1 to about 8.1. In some embodiments, the pH of a formulation comprising a rAAV vector may be about 7.0, about 7.1, about 7.2, about 7.3, about 7.4, about 7.5, about 7.6, about 7.7, about 7.8, about 7.9, about 8.0, about 8.1, about 8.2, about 8.3, about 8.4 or about 8.5. Further, in some embodiments the pH of a formulation comprising a rAAV3B vector is about 7.6.

In some embodiments, the disclosure provides a formulation comprising a rAAV vector with a viscosity between about 0.5 mPa to about 5 mPa, e.g. about 0.5 mPa to about 4 mPa, about 0.5 mPa to about 3 mPa, about 0.5 mPa to about 2 mPa, about 0.5 mPa to about 1.5 mPa, about 1 mPa to about 4 mPa, about 1 mPa to about 3 mPa, about 1 mPa to about 2 mPa or about 1 mPa to about 1.5 mPa. In some embodiments, a formulation comprising a rAAV vector has a viscosity of about 0.5 mPa, about 0.6 mPa, about 0.7 mPa, about 0.8 mPa, about 0.9 mPa, about 1.0 mPa, about 1.1 mPa, about 1.2 mPa, about 1.3 mPa, about 1.4 mPa, about 1.5 mPa, about 1.6 mPa, about 1.7 mPa, about 1.8 mPa, about 1.9 mPa, about 2.0 mPa, about 2.5 mPa, about 3.0 mPa, about 3.5 mPa, about 4.0 mPa, about 4.5 mPa or about 5.0 mPa. In some embodiments, a formulation comprising a rAAV (e.g., rAAV3B) vector has a viscosity of about 1.19 mPa or about 1.193 mPa when measured at 21° C.

In some embodiments, the disclosure provides a formulation comprising a rAAV vector with a density between about 0.5 g/cm³ to about 5 g/cm³, e.g., about 0.5 g/cm³ to about 4 g/cm³, about 0.5 g/cm³ to about 3 g/cm³, about 0.5 g/cm³ to about 2 g/cm³, about 0.5 g/cm³ to about 1.5 g/cm³, about 1 g/cm³ to about 4 g/cm³, about 1 g/cm to about 3 g/cm³, about 1 g/cm³ to about 2 g/cm³ or about 1 g/cm³ to about 1.5 g/cm³.

In some embodiments, a formulation comprising a rAAV vector has a density of about 0.5 g/cm³, about 0.6 g/cm³, about 0.7 g/cm³, about 0.8 g/cm³, about 0.9 g/cm³, about 1.0 g/cm³, about 1.1 g/cm³, about 1.2 g/cm³, about 1.3 g/cm³, about 1.4 g/cm³, about 1.5 g/cm³, about 1.6 g/cm³, about 1.7 g/cm³, about 1.8 g/cm³, about 1.9 g/cm³, about 2.0 g/cm³, about 2.5 g/cm³, about 3.0 g/cm³, about 3.5 g/cm³, about 4.0 g/cm³, about 4.5 g/cm³ or about 5.0 g/cm³. In some embodiments, a formulation comprising a rAAV (e.g., rAAV3B) vector has a density of about 1.03 g/cm³ or about 1.025 g/cm³.

In some embodiments, the disclosure provides a formulation comprising a rAAV vector with a conductivity between about 1 mS/cm to about 40 mS/cm, e.g., about 1 mS/cm to about 35 mS/cm, about 1 mS/cm to about 30 mS/cm, about 1 mS/cm to about 25 mS/cm, about 1 mS/cm to about 20 mS/cm, about 1 mS/cm to about 15 mS/cm, about 1 mS/cm to about 10 mS/cm, about 5 mS/cm to about 40 mS/cm, about 5 mS/cm to about 35 mS/cm, about 5 mS/cm to about 30 mS/cm, about 5 mS/cm to about 25 mS/cm, about 5 mS/cm to about 20 mS/cm, about 5 mS/cm to about 15 mS/cm, about 5 mS/cm to about 10 mS/cm, about 10 mS/cm to about 40 mS/cm, about 10 mS/cm to about 35 mS/cm, about 10 mS/cm to about 30 mS/cm, about 10 mS/cm to about 25 mS/cm, about 10 mS/cm to about 20 mS/cm or about 10 mS/cm to about 15 mS/cm.

In some embodiments, a formulation comprising a rAAV vector has a conductivity of about 1 mS/cm, about 2 mS/cm, about 3 mS/cm, about 4 mS/cm, about 5 mS/cm, about 6 mS/cm, about 7 mS/cm, about 8 mS/cm, about 9 mS/cm, about 10 mS/cm, about 10.5 mS/cm, about 11 mS/cm, about 11.5 mS/cm, about 12 mS/cm, about 12.5 mS/cm, about 13 mS/cm, about 13.5 mS/cm, about 14 mS/cm, about 14.5 mS/cm, about 15 mS/cm, about 15.5 mS/cm, about 16.1 mS/cm, about 16.2 mS/cm, about 16.3 mS/cm, about 16.4 mS/cm, about 16.5 mS/cm, about 16.6 mS/cm, about 16.7 mS/cm, about 16.8 mS/cm, about 16.9 mS/cm, about 17 mS/cm, about 17.1 mS/cm, about 17.2 mS/cm, about 17.3 mS/cm, about 17.4 mS/cm, about 17.5 mS/cm, about 17.6 mS/cm, about 17.7 mS/cm, about 17.8 mS/cm, about 17.9 mS/cm, about 18 mS/cm, about 18.5 mS/cm, about 19 mS/cm, about 19.5 mS/cm, about 20 mS/cm, about 25 mS/cm, about 30 mS/cm, about 35 mS/cm or about 40 mS/cm. In some embodiments, a formulation comprising a rAAV (e.g., rAAV3B) vector has a conductivity of about 16.94 mS/cm.

A pharmaceutical composition may further comprise one or more preservatives such as ascorbic acid (vitamin C), sulfites, sorbates, benzoates, phenol, m-cresol, benzyl alcohol, benzalkonium chloride, phenoxyethanol, and/or parabens (e.g., methyl paraben). In some embodiments, a pharmaceutical composition does not contain any added preservatives.

The present disclosure provides a pharmaceutical composition comprising a rAAV vector and at least one other component selected from the group consisting of a buffer, a salt, a cryoprotectant, a surfactant and a combination thereof, optionally, wherein the vector is a rAAV3B vector. In some embodiments, a pharmaceutical composition comprises a rAAV vector and at least one other component selected from the group consisting of Tris, MgCl₂, sucrose, poloxamer 188 and a combination thereof, optionally wherein the vector is a rAAV3B vector. In some embodiments, a pharmaceutical composition comprises a rAAV vector and 20 mM Tris, about 100 mM MgCl₂, about 4% (w/v) sucrose and about 0.02% (w/v) poloxamer 188 at pH 7.6, optionally, wherein the vector is a rAAV3B vector.

In some embodiments, a formulation comprises about 1E+11 vg/mL to about 1E+15 vg/mL of a rAAV vector. In some embodiments, a formulation comprises about 3.0E+11 vg/mL to about 4.0E+13 vg/mL, e.g., about 3.0E+11 vg/mL to about 3.5E+13 vg/mL about 3.0E+11 vg/mL to about 3.0E+13 vg/mL, about 3.0E+11 vg/mL to about 2.5E+13 vg/mL, about 3.0E+11 vg/mL to about 2.0E+13 vg/mL, about 3.0E+11 vg/mL to about 1.5E+13 vg/mL, about 3.0E+11 vg/mL to about 1.0E+13 vg/mL, about, 3.0E+11 vg/mL to about 5.0E+12 vg/mL, about 3.0E+11 vg/mL to about 1.0E+12 vg/mL, about 5.0E+11 vg/mL to about 4.0E+13 vg/mL about 5.0E+11 vg/mL to about 3.5E+13 vg/mL, about 5.0E+11 vg/mL to about 3.0E+13 vg/mL, about 5.0E+11 vg/mL to about 2.5E+13 vg/mL, about 5.0E+11 vg/mL to about 2.0E+13 vg/mL, about 5.0E+11 vg/mL to about 1.5E+13 vg/mL, about 5.0E+11 vg/mL to about 1.0E+13 vg/mL, about 5.0E+11 vg/mL to about 5.0E+12 vg/mL, about 1.0E+12 vg/mL to about 5.0E+13 vg/mL, about 1.0E+12 vg/mL to about 4.0E+13 vg/mL, about 1.0E+12 vg/mL to about 3.0E+13 vg/mL, about 1.0E+12 vg/mL to about 2.0E+13 vg/mL or about 5.0E+12 vg/mL to about 1.5E+13 vg/mL of a rAAV vector, optionally a rAAV3B vector.

In some embodiments, formulation comprises about 3.5E+11 vg/mL, about 4.0E+11 vg/mL, about 5.0E+11 vg/mL, about 6.0E+11 vg/mL, about 7.0E+11 vg/mL, about 8.0E+11 vg/mL, about 9.0E+11 vg/mL, about 1.0E+12 vg/mL, about 2.0E+12 vg/mL, about 3.0E+12 vg/mL, about 4.0E+12 vg/mL, about 5.0E+12 vg/mL, about 6.0E+12 vg/mL, about 7.0E+12 vg/mL, about 8.0E+12 vg/mL, about 9.0E+12 vg/mL, about 9.1E+12 vg/mL, about 9.2E+12 vg/mL, about 9.3E+12 vg/mL, about 9.4E+12 vg/mL, about 9.5E+12 vg/mL, about 9.6E+12 vg/mL, about 9.7E+12 vg/mL, about 9.8E+12 vg/mL, about 9.5E+12 vg/mL, about 1.0E+13 vg/mL, about 1.1E+13 vg/mL, about 1.2E+13 vg/mL, 1.3E+13 vg/mL, about 1.4E+13 vg/mL, about 1.5E+13 vg/mL, about 1.6E+13 vg/mL, about 1.7E+13 vg/mL, about 1.8E+13 vg/mL, about 1.9E+13 vg/mL, about 2.0E+13 vg/mL, about 2.1E+13 vg/mL, about 2.2E+13 vg/mL, about 2.3E+13 vg/mL, about 2.4E+13 vg/mL, about 2.5E+13 vg/mL, about 3.0E+13 vg/mL, or about 3.5E+13 vg/mL of a rAAV vector, optionally a rAAV3B vector.

In some embodiments, a formulation comprising a rAAV vector comprises about 4E+11 vg/mL, about 2E+12 vg/mL, about 1E+13 vg/mL or about 2E+13 vg/mL optionally wherein the rAAV vector is a rAAV3B vector.

The pharmaceutical compositions may also comprise other reagents that enhance the effectiveness of the pharmaceutical composition. The pharmaceutical composition may contain delivery vehicles such as liposomes, nanocapsules, microparticles, microspheres, lipid particles, and vesicles.

In some exemplary embodiments, the present disclosure provides an improved pharmaceutical composition comprising a rAAV vector at a concentration of about 3E+11 vg/mL to about 3E+13 vg/mL, a buffer at a concentration of about 10 mM to about 30 mM, a salt at a concentration of about 50 mM to about 150 mM, a cryoprotectant at a concentration of about 1.0% (w/v) to about 10% (w/v) and a surfactant at a concentration of about 0.01% (w/v) to about 0.1% (w/v) at a pH of about 7.3 to about 7.9, optionally wherein the rAAV vector comprises a vector genome comprising an ATP7B transgene, or fragment thereof, optionally encoding a polypeptide comprising or consisting of the amino acid sequence of SEQ ID NO:1, and optionally wherein the rAAV vector comprises an AAV3B capsid polypeptide.

In some exemplary embodiments, the present disclosure provides an improved pharmaceutical composition comprising a rAAV vector at a concentration of about 3E+11 vg/mL to about 3E+13 vg/mL, Tris at a concentration of about 10 mM to about 30 mM (e.g., about 20 mM), MgCl₂ at a concentration of about 50 mM to about 150 mM (e.g., about 100 mM), sucrose at a concentration of about 1.0% (w/v) to about 10% (w/v) (e.g., about 4%) and poloxamer 188 at a concentration of about 0.01% (w/v) to about 0.1% (w/v) (e.g., about 0.02%) at a pH of about 7.3 to about 7.9 (e.g., about 7.6), optionally wherein the rAAV vector comprises a vector genome comprising or consisting of an ATP7B transgene, or fragment thereof, optionally encoding a polypeptide comprising or consisting of the amino acid sequence of SEQ ID NO:1, an AAT promoter comprising or consisting of the nucleic acid sequence of SEQ ID NO:3, a polyA signal sequence comprising or consisting of the nucleic acid sequence of SEQ ID NO:4 flanked by AAV2 ITR sequences, and optionally wherein the rAAV vector comprises an AAV3B capsid polypeptide.

### V. Use of Recombinant AAV Formulations

The pharmaceutical compositions of the present disclosure may be used for the treatment of Wilson Disease (WD), a rare, debilitating, life-threatening disorder of copper homeostasis that is due to mutations in the APT7B gene. In some embodiments, administration of a pharmaceutical composition comprising the rAAV vector as disclosed herein (e.g., rAAV3B vector with vector genome comprising a nucleic acid encoding a copper-transporting ATPase 2 with a deletion of metal binding sites (MBS) 1-4 and/or encoding a copper transporting ATPase 2 comprising the amino acid sequence of SEQ ID NO:1) to a subject with WD may result in one or more treatment effects including: restoration of cooper metabolism (e.g., as measured by increased fecal and renal excretion of cooper, decreased serum cooper and decreased hepatic copper), increased serum ceruloplasmin levels, decreased urinary copper, decreased hepatic copper, reversal of liver injury (e.g., normalization of hepatic function and enzyme parameters), reduced or no need for treatment with zinc salts and/or chelation therapy, serum alanine transaminase levels less than/equal to the upper limit of normal, serum aspartate transaminase levels less than/equal to the upper limit of normal, and decreased neurological symptoms.

A pharmaceutical composition of the invention may be supplied in an article of manufacture (e.g., a kit) comprising a container for holding a composition described herein and instructions for use. In some embodiments, a container for holding a composition is a vial. In some embodiments, a vial is a cyclic-olefin copolymer vial. In some embodiments, a vial is a sterile high-density polyethylene (HDPE) vial. In some embodiments, a vial contains about 1E+11 vg/mL to about 1E+15 vg/mL of rAAV vector in 0.5-50 mL (e.g., 1-10 mL).

In some embodiments, a pharmaceutical composition of the disclosure is administered at a dose volume of about 1 mL to about 50 mL, e.g., about 1 mL to about 5 mL, about 1 mL to about 10 mL, about 1 mL to about 15 mL, about 1 mL to about 20 mL, about 1 mL to about 25 mL, about 1 mL to about 30 mL, about 1 mL to about 35 mL, about 1 mL to about 40 mL, or about 1 mL to about 45 mL.

In some embodiments, a pharmaceutical composition of the disclosure is administered at a dose volume of about, 1 mL, about 1.5 mL, about 2 mL, about 2.5 mL, about 3 mL, about 3.5 mL, about 4 mL, about 4.1 mL, about 4.2 mL, about 4.3 mL, about 4.4 mL, about 4.5 mL, about 4.6 mL, about 4.7 mL, about 4.8 mL, about 4.9 mL, about 5.0 mL, about 5.1 mL, about 5.2 mL, about 5.3 mL, about 5.4 mL, about 5.5 mL, about 5.6 mL, about 5.7 mL, about 5.8 mL, about 5.9 mL, about 6.0 mL, about 6.1 mL, about 6.2 mL, about 6.3 mL, about 6.4 mL, about 6.5 mL, about 6.6 mL, about 6.7 mL, about 6.8 mL, about 6.9 mL, about 7.0 mL, about 7.1 mL, about 7.2 mL, about 7.3 mL, about 7.4 mL, about 7.5 mL, about 7.6 mL, about 7.7 mL, about 7.8 mL, about 7.9 mL, about 8.0 mL, about 8.1 mL, about 8.2 mL, about 8.3 mL, about 8.4 mL, about 8.5 mL, about 8.6 mL, about 8.7 mL, about 8.9 mL, about 9.0 mL, about 9.1 mL, about 9.2 mL, about 9.3 mL, about 9.4 mL, about 9.5 mL, about 9.6 mL, about 9.7 mL, about 9.8 mL, about 9.9 mL, about 10.0 mL, about 10.5 mL, about 15 mL, about 20 mL, about 25 mL, about 30 mL, about 35 mL, about 40 mL, about 45 mL or about 50 mL.

A pharmaceutical composition of the disclosure may be administered to a patient once, or more than once. For example, a composition may be administered to a patient at an interval of no less than 1 month, 3 months, 6 months, 9 months, or one year. In some embodiments, a composition may be administered to a patient with an interval of no less than two, five, seven, ten, or fifteen years. A pharmaceutical composition may be provided to a patient in need thereof through a route appropriate for the disease to be treated. For example, a composition may be administered parenterally, including by intravenous injection, intraarterially injection, intracranial injection, intraperitoneal injection, portal vein injection, intramuscular injection, intrathecal administration, or subcutaneous injection. For example, a pharmaceutical composition comprising a rAAV vector for the expression of a therapeutic transgene may be provided intravenously to a patient in need thereof at a dose of about 5E+11 vg/kg to about 1E+14 vg/kg, e.g., such as about 1E+12 vg/kg to about 1E+13 vg/kg. In some embodiments, a pharmaceutical composition of the disclosure is administered to a patient by intravenous infusion over a period of time, for example over a period of 30 minutes to 5 hours.

In some embodiments, a pharmaceutical composition comprising a rAAV3B for the expression of an ATP7B transgene is provided intravenously to a Wilson disease patient at a dose of about 5E+11 vg/kg to about 1E+14 vg/kg or about 1E+12 vg/kg to about 1E+13 vg/kg.

In some embodiments, a pharmaceutical composition of the present disclosure is administered to patients with WD who are greater than 12 years of age. In some embodiments, a pharmaceutical composition of the present disclosure is administered to subjects with WD who are age 18-60, optionally who are on a standard of care therapy, e.g., chelation therapy.

In some embodiments, a composition of the disclosure is lyophilized and/or has been subjected to lyophilization. In some embodiments, a composition of the disclosure is not lyophilized and has not been subject to lyophilization.

In some aspects, a composition comprising a rAAV vector is provided for the manufacture of a medicament for the treatment of a disease associated with loss or reduced expression of the ATP7B gene (e.g., Wilson disease) as described herein.

In some aspects, a composition comprising a rAAV vector is provided for use in a method of treatment of a disease associated with loss or reduced expression of the ATP7B gene (e.g., Wilson disease) as described herein.

Unless otherwise defined herein, scientific and technical terms used in connection with the present disclosure shall have the meanings that are commonly understood by those of ordinary skill in the art. All publications and other references mentioned herein are incorporated by reference in their entirety. Although a number of documents are cited herein, this citation does not constitute an admission that any of these documents form part of the common general knowledge in the art.

### EMBODIMENTS

Those skilled in the art will recognize or be able to ascertain using no more than routine experimentation, many equivalents to the specific embodiments of the invention described herein. Such equivalents are intended to be encompassed by the following embodiments (E).

E1. A pharmaceutical composition comprising:
a recombinant adeno-associated virus (rAAV) vector,
a buffer;
a salt;
a cryoprotectant; and
a surfactant,
wherein the pH of the pharmaceutical composition is from about 7.0 to about 8.5.
   E2. The composition of E1, wherein the concentration of the buffer is about 1 mM to 500 mM Tris, e.g., about 1 mM to about 450 mM, about 1 mM to about 400 mM, about 1 mM to about 350 mM, about 1 mM to about 300 mM, about 1 mM to about 250 mM, about 1 mM to about 200 mM, about 1 mM to about 150 mM, about 1 mM to about 100 mM, about 1 mM to 75 mM, about 1 mM to 50 mM, about 1 mM to 25 mM, about 5 mM to about 30 mM, about 10 mM to about 30 mM or about 14 mM to about 26 mM.
   E3. The composition of E1 or E2, wherein the concentration of the buffer is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM or about 500 mM.
   E4. The composition of any one of E1-E3, wherein the concentration of the buffer is about 20 mM.
   E5. The composition of any one of E1-E4, wherein the buffer is selected from the group consisting of acetate, succinate (e.g., disodium succinate hexahydrate), succinic acid, gluconate, citrate, histidine, acetic acid, phosphate, phosphoric acid, ascorbate, ascorbic acid, tartaric acid, malate, maleic acid, glycine, lactate, lactic acid, bicarbonate, carbonic acid, sodium benzoate, benzoic acid, edetate, imidazole, Tris (e.g., Tris base, Tris HCL or both), and a combination thereof.
   E6. The composition of any one of E1-E5, wherein the buffer is Tris.
   E7. The composition of any one of E1-E6, wherein the Tris is Tris base, Tris HCl or a combination of Tris base and Tris HCl.
   E8. The composition of any one of E1-E7, wherein the concentration of the salt is about 1 mM to about 500 mM, e.g., about 1 mM to about 450 mM, about 1 mM to about 400 mM, about 1 mM to about 350 mM, about 1 mM to about 300 mM, about 1 mM to about 250 mM, about 1 mM to about 200 mM, about 1 mM to about 150 mM, about 1 mM to about 100 mM, about 10 mM to about 450 mM, about 10 mM to about 400 mM, about 10 mM to about 350 mM, about 10 mM to about 300 mM, about 10 mM to about 250 mM, about 10 mM to about 200 mM, about 10 mM to 175 mM, about 10 mM to 150 mM, about 10 mM to 125 mM, about 50 mM to about 450 mM, about 50 mM to about 400 mM, about 50 mM to about 350 mM, about 50 mM to about 300 mM, about 50 mM to about 250 mM, about 50 mM to about 200 mM, about 50 mM to about 150 mM or about 75 mM to about 450 mM, about 75 mM to about 400 mM, about 75 mM to about 350 mM, about 75 mM to about 300 mM, about 75 mM to about 250 mM, about 75 mM to about 200 mM, about 75 mM to about 150 mM, about 75 mM to about 125 mM or about 70 mM to about 130 mM.
   E9. The composition of any one of E1-E8, wherein the concentration of the salt is about 10 mM, about 20 mM, about 30 mM, about 40 mM, about 50 mM, about 60 mM, about 70 mM, about 80 mM, about 90 mM, about 91 mM, about 92 mM, about 93 mM, about 94 mM, about 95 mM, about 96 mM, about 97 mM, about 98 mM, about 99 mM, about 100 mM, about 101 mM, about 102 mM, about 103 mM, about 104 mM, about 105 mM, about 106 mM, about 107 mM, about 108 mM, about 109 mM, about 110 mM, about 120 mM, about 130 mM, about 140 mM, about 150 mM, about 160 mM, about 170 mM, about 180 mM, about 190 mM, about 200 mM, about 225 mM, about 250 mM, about 275 mM, about 300 mM, about 325 mM, about 350 mM, about 375 mM, about 400 mM, about 425 mM, about 450 mM, about 475 mM or about 500 mM.
   E10. The composition of any one of E1-E9, wherein the concentration of the salt is about 100 mM.
   E11. The composition of any one of E1-10, wherein the salt is selected from the group consisting of MgCl₂, MgSO₄, CaCl₂ and a combination thereof.
   E12. The composition of any one of E1-E11, wherein the salt is MgCl₂.
   E13. The composition of any one of E1-E12, wherein the concentration of the cryoprotectant is about 0.1% to about 20% (w/v), e.g., about 0.1% to about 19% (w/v), about 0.1% to about 18% (w/v), about 0.1% to about 17% (w/v), about 0.1% to about 16% (w/v), about 0.1% to about 15% (w/v), about 0.1% to about 14% (w/v), about 0.1% to about 13% (w/v), about 0.1% to about 12% (w/v), about 0.1% to about 11% (w/v), about 0.1% to about 10% (w/v), about 0.1% to about 9% (w/v), about 0.1% to about 8% (w/v), about 0.1% to about 7% (w/v), about 0.1% to about 6% (w/v), about 0.1% to about 5% (w/v), about 0.1% to about 4% (w/v), about 0.1% to about 3% (w/v), about 0.1% to about 2% (w/v), about 0.1% to about 1% (w/v), about 1% to about 19% (w/v), about 1% to about 18% (w/v), about 1% to about 17% (w/v), about 1% to about 16% (w/v), about 1% to about 15% (w/v), about 1% to about 14% (w/v), about 1% to about 13% (w/v), about 1% to about 12% (w/v), about 1% to about 11% (w/v), about 1% to about 10% (w/v), about 1% to about 8% (w/v), about 1% to about 6% (w/v), about 2% to about 6% (w/v) or about 2.8% to about 5.2% (w/v).
   E14. The composition of any one of E1-E13, wherein the concentration of the cryoprotectant is about 1% (w/v), about 2% (w/v), about 2.5% (w/v), about 2.6% (w/v), about 2.7% (w/v), about 2.8% (w/v), about 2.9% (w/v), about 3% (w/v), about 3.1% (w/v), about 3.2% (w/v), about 3.3% (w/v), about 3.4% (w/v), about 3.5% (w/v), about 3.6% (w/v), about 3.7% (w/v), about 3.8% (w/v), about 3.9% (w/v), about 4% (w/v), about 4.1% (w/v), about 4.2% (w/v), about 4.3% (w/v), about 4.4% (w/v), about 4.6% (w/v), about 4.7% (w/v), about 4.8% (w/v), about 4.9% (w/v), about 5% (w/v), about 5.1% (w/v), about 5.2% (w/v), about 5.3% (w/v), about 5.4%, about 5.5% (w/v), about 6% (w/v), about 7% (w/v), about 8% (w/v), about 9% (w/v), about 10% (w/v), about 11% (w/v), about 12% (w/v), about 13% (w/v), about 14% (w/v), about 15% (w/v), about 16% (w/v), about 17% (w/v), about 18% (w/v), about 19% (w/v) or about 20% (w/v).
   E15. The composition of any one of E1-E14, wherein the concentration of the cryoprotectant is about 4% (w/v).
   E16. The composition of any one of E1-E15, wherein the cryoprotectant is selected from the group consisting of sugar (e.g., dextrose, lactose, glucose, fructose, maltose, mannose, sorbose, sucrose, trehalose, xylose), sugar alcohol (e.g., erythritol, ethylene glycol, glycerol, isomalt, inositol, lactitol, maltitol, mannitol, sorbitol, xylitol), amino acid (e.g., glycine, histidine, arginine), polypeptide, protein (e.g., albumin, gelatine), polymer (e.g., dextran, polyvinyl pyrrolidone, polyvinyl alcohol, propylene glycol, polyethylene glycol), water-soluble glucan, and a combination thereof.
   E17. The composition of any one of E1-E16, wherein the cryoprotectant is sucrose.
   E18. The composition of any one of E1-E17, wherein the concentration of the surfactant is about 0.001% (w/v) to about 0.5% (w/v), e.g., about 0.001% to about 0.4% (w/v), about 0.001% to about 0.3% (w/v), about 0.001% to about 0.2% (w/v), about 0.001% to about 0.1% (w/v), about 0.002% to about 0.5% (w/v), about 0.002% to about 0.4% (w/v), about 0.002% to about 0.3% (w/v), about 0.002% to about 0.2% (w/v), about 0.002% to about 0.15% (w/v), about 0.002% to about 0.1% (w/v), about 0.002% to about 0.05% (w/v), about 0.002% to about 0.04% (w/v), about 0.002% to about 0.03% (w/v), about 0.005% to about 0.5% (w/v), about 0.005% to about 0.4% (w/v), about 0.005% to about 0.3% (w/v), about 0.005% to about 0.2% (w/v), about 0.005% to about 0.15% (w/v), about 0.005% to about 0.1% (w/v), about 0.005% to about 0.05% (w/v), about 0.005% to about 0.04% (w/v), about 0.005% to about 0.03% (w/v), about 0.01% to about 0.5% (w/v), about 0.01% to about 0.4% (w/v), about 0.01% to about 0.3% (w/v), about 0.01% to about 0.2% (w/v), about 0.01% to about 0.1% (w/v), about 0.01% to about 0.05% (w/v), about 0.01% to about 0.03% (w/v) or about 0.014% to about 0.026%.
   E19. The composition of any of E1-E18, wherein the concentration of the surfactant is about 0.005% (w/v), about 0.006% (w/v), about 0.007% (w/v), about 0.008% (w/v), about 0.009% (w/v), about 0.01% (w/v), about 0.014% (w/v), about 0.015% (w/v), about 0.016% (w/v), about 0.017% (w/v), about 0.018% (w/v), about 0.019% (w/v), about 0.02% (w/v), about 0.021% (w/v), about 0.022% (w/v), about 0.023% (w/v), about 0.024% (w/v), about 0.025% (w/v), about 0.026% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v) or about 0.5% (w/v).
   E20. The composition of any one of E1-E19, wherein the concentration of the surfactant is about 0.02% (w/v).
   E21. The composition of any one of E1-E20, wherein the surfactant is selected from the group consisting of a polysorbate, a poloxamer, triton, sodium dodecyl sulfate (SDS), sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol and a combination thereof.
   E22. The composition of any one of E1-E21, wherein the surfactant is poloxamer 188.
   E23. The composition of any one of E1-E22, wherein the pH of the composition is about 7.0 to about 8.5, e.g., about 7.1 to about 8.1.
   E24. The composition of any one of E1-E23, wherein the pH of the compositions is about 7.0, about 7.1, about 7.2, about 7.3, about 7.4, about 7.5, about 7.6, about 7.7, about 7.8, about 7.9, about 8.0, about 8.1, about 8.2, about 8.3, about 8.4 or about 8.5.
   E25. The composition of any one of E1-E24, wherein the pH of the composition is about 7.6 +/−0.5.
   E26. The composition of any one of E1-E25, wherein the viscosity of the composition is about 0.5 mPa to about 5 mPa.
   E27. The composition of any of E1-E26, wherein the viscosity of the composition is about 0.5 mPa, about 0.6 mPa, about 0.7 mPa, about 0.8 mPa, about 0.9 mPa, about 1.0 mPa, about 1.1 mPa, about 1.2 mPa, about 1.3 mPa, about 1.4 mPa, about 1.5 mPa, about 1.6 mPa, about 1.7 mPa, about 1.8 mPa, about 1.9 mPa, about 2.0 mPa, about 2.5 mPa, about 3.0 mPa, about 3.5 mPa, about 4.0 mPa, about 4.5 mPa or about 5.0 mPa
   E28. The composition of any one of E1-E27, wherein the viscosity of the composition is about 1 mPa to about 1.5 mPa, optionally about 1.19 mPa or about 1.93 mPa.
   E29. The composition of any one of E1-E28, wherein the density of the composition is about 0.5 g/cm³ to about 5 g/cm³.
   E30. The composition of any one of E1-E29, wherein the density of the composition is about 0.5 g/cm³, about 0.6 g/cm³, about 0.7 g/cm³, about 0.8 g/cm³, about 0.9 g/cm³, about 1.0 g/cm³, about 1.1 g/cm³, about 1.2 g/cm³, about 1.3 g/cm³, about 1.4 g/cm³, about 1.5 g/cm³, about 1.6 g/cm³, about 1.7 g/cm³, about 1.8 g/cm³, about 1.9 g/cm³, about 2.0 g/cm³, about 2.5 g/cm³, about 3.0 g/cm³, about 3.5 g/cm³, about 4.0 g/cm³, about 4.5 g/cm³ or about 5.0 g/cm³.
   E31. The composition of any one of E1-E30, wherein the density of the composition is about 1 g/cm³ to about 1.5 g/cm³, optionally about 1.03 g/cm³ or about 1.025 g/cm³.
   E32. The composition of any one of E1-E31, wherein the conductivity of the composition is about 1 mS/cm to about 40 mS/cm.
   E33. The composition of any one of E1-E32, wherein the conductivity of the composition is about 1 mS/cm, about 2 mS/cm, about 3 mS/cm, about 4 mS/cm, about 5 mS/cm, about 6 mS/cm, about 7 mS/cm, about 8 mS/cm, about 9 mS/cm, about 10 mS/cm, about 10.5 mS/cm, about 11 mS/cm, about 11.5 mS/cm, about 12 mS/cm, about 12.5 mS/cm, about 13 mS/cm, about 13.5 mS/cm, about 14 mS/cm, about 14.5 mS/cm, about 15 mS/cm, about 15.5 mS/cm, about 16.1 mS/cm, about 16.2 mS/cm, about 16.3 mS/cm, about 16.4 mS/cm, about 16.5 mS/cm, about 16.6 mS/cm, about 16.7 mS/cm, about 16.8 mS/cm, about 16.9 mS/cm, about 17 mS/cm, about 17.1 mS/cm, about 17.2 mS/cm, about 17.3 mS/cm, about 17.4 mS/cm, about 17.5 mS/cm, about 17.6 mS/cm, about 17.7 mS/cm, about 17.8 mS/cm, about 17.9 mS/cm, about 18 mS/cm, about 18.5 mS/cm, about 19 mS/cm, about 19.5 mS/cm, about 20 mS/cm, about 25 mS/cm, about 30 mS/cm, about 35 mS/cm or about 40 mS/cm.
   E34. The composition of any one of E1-E33, wherein the conductivity of the composition is about 10 mS/cm to about 20 mS/cm, optionally about 16.9 mS/cm or about 16.94 mS/cm.
   E35. The composition of any one of E1-E34, wherein the rAAV vector comprises a capsid polypeptide of an AAV serotype selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1, RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15.
   E36. The composition of any one of E1-E35, wherein the rAAV vector comprises a capsid polypeptide of the AAV3B serotype.
   E37. The composition of E36, wherein the capsid polypeptide of the AAV3B serotype comprises or consists of the amino acid sequence of SEQ ID NO:10.
   E38. The composition of E36 or E37, wherein the capsid polypeptide of the AVV3B serotype is encoded by the nucleic acid sequence of SEQ ID NO:11.
   E39. The composition of any one of E1-E38, wherein the rAAV vector comprises a vector genome comprising an ATP7B transgene.
   E40. The composition of E39, wherein the ATP7B transgene i) comprises or consists of the nucleic acid sequence of SEQ ID NO:2, ii) encodes a copper transporting ATPase 2 with a deletion of metal binding sites (MBS) 1-4 and/or iii) encodes a copper transporting ATPase 2 comprising or consisting of the amino acid sequence of SEQ ID NO:1.
   E41. The composition of E39 or E40, wherein the rAAV vector genome further comprises a nucleic acid sequence of a promoter, a polyA signal sequence and at least one ITR sequence of an AAV.
   E42. The composition of E39-E40, wherein the rAAV vector genome further comprises a nucleic acid sequence of an AAT promoter, a polyA signal sequence from a rabbit ß-globin gene and at least one ITR sequence of the AAV2 serotype.
   E43. The composition of E41 or E42, wherein the at least one ITR sequence is a 5′ ITR and a 3′ ITR.
   E44. The composition of any one of E41-E43, wherein the promoter comprises a nucleic acid sequence that is, or is at least, or is at most 80%, 85%, 90%, 95%, 98%, 99% or 100% identical to SEQ ID NO:3.
   E45. The composition of any one of E41-E44, wherein the polyA signal sequence comprises a nucleic acid sequence that is, or is at least, or is at most 80%, 85%, 90%, 95%, 98%, 99% or 100% identical to SEQ ID NO:4.
   E46. The composition of any one of E41-E45, wherein the at least one ITR comprises a nucleic acid sequence that is, or is at least, or is at most 80%, 85%, 90%, 95%, 98%, 99% or 100% identical to a nucleic acid sequence selected from the group consisting of SEQ ID NO:5-8 and a combination thereof.
   E47. The composition of any one of E1-E46, wherein the composition comprises about 1E+11 vg/mL to about 1E+15 vg/mL of the rAAV vector, optionally, wherein the rAAV vector is a rAAV3B vector.
   E48. The composition of any one of E1-E47, wherein the composition comprises about 3.0E+11 vg/mL to about 3.0E+13 vg/mL of a rAAV vector, optionally, wherein the rAAV vector is a rAAV3B vector.
   E49. The composition of any one of E1-E48, wherein the composition comprises about 3.0E+11 vg/mL to about 3.5E+13 vg/mL, about 3.0E+11 vg/mL to about 3.0E+13 vg/mL, about 3.0E+11 vg/mL to about 2.5E+13 vg/mL, about 3.0E+11 vg/mL to about 2.0E+13 vg/mL, about 3.0E+11 vg/mL to about 1.5E+13 vg/mL, about 3.0E+11 vg/mL to about 1.0E+13 vg/mL, about, 3.0E+11 vg/mL to about 5.0E+12 vg/mL, about 3.0E+11 vg/mL to about 1.0E+12 vg/mL, about 5.0E+11 vg/mL to about 4.0E+13 vg/mL about 5.0E+11 vg/mL to about 3.5E+13 vg/mL, about 5.0E+11 vg/mL to about 3.0E+13 vg/mL, about 5.0E+11 vg/mL to about 2.5E+13 vg/mL, about 5.0E+11 vg/mL to about 2.0E+13 vg/mL, about 5.0E+11 vg/mL to about 1.5E+13 vg/mL, about 5.0E+11 vg/mL to about 1.0E+13 vg/mL, about 5.0E+11 vg/mL to about 5.0E+12 vg/mL, about 1.0E+12 vg/mL to about 5.0E+13 vg/mL, about 1.0E+12 vg/mL to about 4.0E+13 vg/mL, about 1.0E+12 vg/mL to about 3.0E+13 vg/mL, about 1.0E+12 vg/mL to about 2.0E+13 vg/mL or about 5.0E+12 vg/mL to about 1.5E+13 vg/mL of a rAAV vector.
   E50. The composition of any one of E1-E49, wherein the composition comprises about 3.0E+11 vg/mL, about 3.5E+11 vg/mL, about 4.0E+11 vg/mL, about 5.0E+11 vg/mL, about 6.0E+11 vg/mL, about 7.0E+11 vg/mL, about 8.0E+11 vg/mL, about 9.0E+11 vg/mL, about 1.0E+12 vg/mL, about 2.0E+12 vg/mL, about 3.0E+12 vg/mL, about 4.0E+12 vg/mL, about 5.0E+12 vg/mL, about 6.0E+12 vg/mL, about 7.0E+12 vg/mL, about 8.0E+12 vg/mL, about 9.0E+12 vg/mL, about 9.1E+12 vg/mL, about 9.2E+12 vg/mL, about 9.3E+12 vg/mL, about 9.4E+12 vg/mL, about 9.5E+12 vg/mL, about 9.6E+12 vg/mL, about 9.7E+12 vg/mL, about 9.8E+12 vg/mL, about 9.5E+12 vg/mL, about 1.0E+13 vg/mL, about 1.1E+13 vg/mL, about 1.2E+13 vg/mL, about 1.3E+13 vg/mL, about 1.4E+13 vg/mL, about 1.5E+13 vg/mL, about 1.6E+13 vg/mL, about 1.7E+13 vg/mL, about 1.8E+13 vg/mL, about 1.9E+13 vg/mL, about 2.0E+13 vg/mL, about 2.1E+13 vg/mL, about 2.2E+13 vg/mL, about 2.3E+13 vg/mL, about 2.4E+13 vg/mL, about 2.5E+13 vg/mL, about 3.0E+13 vg/mL, about 3.5E+13 vg/mL or about 4.0E+13 vg/mL of a rAAV vector, optionally, wherein the rAAV vector is a rAAV3B vector.
   E51. The composition of any one of E1-E50, wherein the composition comprises about 4E+11 vg/mL, about 2E+12 vg/mL, about 1E+13 vg/mL or about 2E+13 vg/mL, optionally, wherein the rAAV vector is a rAAV3B vector.
   E52. A pharmaceutical composition comprising
a recombinant adeno-associated virus (rAAV) vector;
about 1 mM to about 500 mM Tris, optionally about 20 mM Tris;
about 1 mM to about 500 mM MgCl₂, optionally about 100 mM MgCl₂;
about 0.1% to about 20% (w/v) sucrose, optionally about 4% (w/v) sucrose; and
about 0.001% to about 0.5% (w/v) poloxamer 188, optionally about 0.02% (w/v) poloxamer 188,
   wherein the pH of composition is from about 7.1 to about 8.1.
   E53. A pharmaceutical composition comprising
a recombinant adeno-associated virus (rAAV) vector;
about 14 mM to about 26 mM Tris;
about 70 mM to about 130 mM MgCl₂;
about 2.8% to about 5.2% (w/v) sucrose; and
about 0.014% to about 0.026% (w/v) poloxamer 188,
   wherein the pH of composition is from about 7.1 to about 8.1.
   E54. The composition of E52 or E53, wherein the composition comprises about 3.0E+11 vg/mL to about 3.0E+13 vg/mL of rAAV vector.
   E55. The composition of any one of E52-E54, wherein the composition comprises about 4E+11 vg/mL, about 2E+12 vg/mL, about 1E+13 vg/mL or about 2E+13 vg/mL of a rAAV vector.
   E56. The composition of any one of E52-E55, wherein the rAAV vector comprises a polypeptide of an AAV serotype selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1, RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15.
   E57. The composition of any one of E52-E56, wherein the rAAV vector comprises a capsid polypeptide of the AAV3B serotype.
   E58. The composition of E57, wherein the capsid polypeptide of the AAV3B serotype comprises or consists of the amino acid sequence of SEQ ID NO:10.
   E59. The composition of E57 or E58, wherein the capsid polypeptide of the AAV3B serotype is encoded by the nucleic acid sequence of SEQ ID NO:11.
   E60. The composition of any one of E52-E59, wherein the rAAV vector comprises a vector genome comprising a transgene and one or more regulatory elements for the expression of the transgene, optionally, wherein the transgene is ATP7B, or a fragment thereof.
   E61. The composition of E60, wherein the ATP7B transgene i) comprises or consists of the nucleic acid sequence of SEQ ID NO:2, ii) encodes a copper transporting ATPase 2 with a deletion of metal binding sites (MBS) 1-4 and/or iii) encodes a copper transporting ATPase 2 comprising or consisting of the amino acid sequence of SEQ ID NO:1.
   E62. The composition of E60 or E61, wherein the one or more regulatory elements are selected from the group consisting of a promoter, a polyA signal sequence, at least one ITR sequence and a combination thereof.
   E63. The composition of E60 or E62, wherein the one or more regulatory elements are selected from the group consisting of an α1-antitrypsin (AAT) promoter, a polyA signal sequence from a rabbit β-globin gene, at least one ITR sequence and a combination thereof.
   E64. The composition of E62 or E63, wherein the at least one ITR sequence is of the AAV2 serotype.
   E65. The composition of any one of E62 to E64, wherein the at least on ITR sequence is a 5′ ITR and a 3′ ITR.
   E66. The composition of any one of E62-E65, wherein the promoter comprises a nucleic acid sequence that is, or is at least, or is at most 80%, 85%, 90%, 95%, 98%, 99% or 100% identical to SEQ ID NO:3.
   E67. The composition of any one of E62-E66, wherein the polyA signal sequence comprises a nucleic acid sequence that is, or is at least, or is at most 80%, 85%, 90%, 95%, 98%, 99% or 100% identical to SEQ ID NO:4.
   E68. The composition of any one of E62-E67, wherein the at least one ITR comprises a nucleic acid sequence that is, or is at least, or is at most 80%, 85%, 90%, 95%, 98%, 99% or 100% identical a nucleic acid sequence selected from the group consisting of SEQ ID NO:5-8 and a combination thereof.
   E69. The composition of any one of E52-E68, wherein the rAAV vector comprises a vector genome that is, or is at least, or is at most 80%, 85%, 90%, 95%, 98%, 99% or 100% identical to any one of SEQ ID NO:9.
   E70. The composition of any one of claims E52-E69, wherein the rAAV vector comprises a vector genome comprising or consisting of the nucleic acid sequence of SEQ ID NO:9.
   E71. A pharmaceutical composition comprising
a recombinant adeno-associated virus (rAAV) vector comprising a capsid polypeptide of the AAV3B serotype and a vector genome comprising a transgene;
about 14 mM to about 26 mM, optionally about 20 mM Tris;
about 70 mM to about 130 mM, optionally about 100 mM MgCl₂;
about 2.8% (w/v) to about 5.2% (w/v), optionally about 4% (w/v) sucrose; and
about 0.14% (w/v) to about 0.026% (w/v), optionally about 0.02% (w/v) poloxamer 188,
   wherein the pH of composition is from about 7.1 to about 8.1.
   E72. A pharmaceutical composition comprising
a recombinant adeno-associated virus (rAAV) vector comprising a capsid polypeptide of the AAV3B serotype and a vector genome comprising a transgene encoding a copper transporting ATPase 2 protein, or fragment thereof;
about 14 mM to about 26 mM, optionally about 20 mM Tris;
about 70 mM to about 130 mM, optionally about 100 mM MgCl₂;
about 2.8% (w/v) to about 5.2% (w/v), optionally about 4% (w/v) sucrose; and
about 0.14% (w/v) to about 0.026% (w/v), optionally about 0.02% (w/v) poloxamer 188,
   wherein the pH of composition is from about 7.1 to about 8.1.
   E73. A pharmaceutical composition comprising
a recombinant adeno-associated virus (rAAV) vector comprising a capsid polypeptide of the AAV3B serotype and a vector genome comprising a transgene encoding a copper transporting ATPase 2 protein, or fragment thereof;
about 20 mM Tris;
about 100 mM MgCl₂;
about 4% (w/v) sucrose; and
about 0.02% (w/v) poloxamer 188,
   wherein the pH of composition is from about 7.1 to about 8.1.
   E74. The composition of any one of E71-E73, wherein the transgene i) comprises or consists of the nucleic acid sequence of SEQ ID NO:2, ii) encodes a copper transporting ATPase 2 with a deletion of metal binding sites (MBS) 1-4 and/or iii) encodes a copper transporting ATPase 2 comprising or consisting of the amino acid sequence of SEQ ID NO:1.
   E75. The composition of any one of E71-E74, wherein the transgene comprises a nucleic acid sequence that is, or is at least, or is at most 80%, 85%, 90%, 95%, 98%, 99% or 100% identical to SEQ ID NO:2.
   E76. The composition of any one of E71-E75, wherein the vector genome further comprises at least one element selected from the group consisting of a promoter, a polyA signal sequence, at least one ITR and a combination thereof.
   E77. The composition of E76, wherein the promoter is an α1-antitrypsin (AAT) promoter, a portion of an AAT promoter, or a liver specific promoter.
   E78. The composition of E76 or E77, wherein the promoter comprises a nucleic acid sequence that is, or is at least, or is at most 80%, 85%, 90%, 95%, 98%, 99% or 100% identical to SEQ ID NO:3.
   E79. The composition of any one of E76-E78, wherein the promoter comprises or consists of a nucleic acid sequence of SEQ ID NO:3.
   E80. The composition of any one of E76-E79, wherein the polyA signal sequence is from a rabbit β-globin gene.
   E81. The composition of any one of E76-E80, wherein the poly A signal sequence comprises a nucleic acid sequence that is, or is at least, or is at most 80%, 85%, 90%, 95%, 98%, 99% or 100% identical to SEQ ID NO:4.
   E82. The composition of any one of E76-E82, wherein the polyA signal sequence comprises or consists of a nucleic acid sequence of SEQ ID NO:4.
   E83. The composition of any one of E76-E82, wherein the at least one ITR sequence is an AAV2 ITR.
   E84. The composition of any one of E76-E83, wherein the at least one ITR sequence is a 5′ ITR and a 3′ ITR.
   E85. The composition of any one of E76-E84, wherein the at least one ITR comprises a nucleic acid sequence that is, or is at least, or is at most 80%, 85%, 90%, 95%, 98%, 99% or 100% identical to any one of SEQ ID NO:5-8.
   E86. The composition of any one of E76-E85, wherein the at least one ITR sequence comprises or consists of a nucleic acid sequence of any one of SEQ ID NO:5-8.
   E87. The composition of any one of E71-E86, wherein the vector genome comprises a nucleic acid sequence that is, or is at least, or is at most 80%, 85%, 90%, 95%, 98%, 99% or 100% identical to SEQ ID NO:9.
   E88. The composition of any one of E72-E87, wherein the vector genome comprises or consists of the nucleic acid of SEQ ID NO:9.
   E89. A pharmaceutical composition comprising
a recombinant adeno-associated virus (rAAV) vector comprising a capsid polypeptide of the AAV3B serotype and a vector genome comprising a transgene encoding a copper transporting ATPase 2 comprising or consisting of the amino acid sequence of SEQ ID NO:1;
about 20 mM Tris;
about 100 mM MgCl₂;
about 4% (w/v) sucrose; and
about 0.02% (w/v) poloxamer 188,
   wherein the pH of composition is from about 7.1 to about 8.1.
   E90. The composition of E89, wherein the vector genome further comprises a promoter comprising or consisting of the nucleic acid sequence of SEQ ID NO:3, a polyA signal sequence comprising or consisting of the nucleic acid sequence of SEQ ID NO:4, and a 5′ ITR and a 3′ITR sequence.
   E91. The composition of E90, wherein the 5′ ITR and/or the 3′ ITR sequence comprises or consists of the nucleic acid sequence of any one of SEQ ID NO:5-8.
   E92. The composition of E89 or E90, wherein the vector genome comprises or consists of a nucleic acid sequence of SEQ ID NO:9.
   E93. The composition of any one of E1-E92, wherein the pharmaceutical composition is lyophilized or is not lyophilized.
   E94. A vial comprising 1 mL to 10 mL of the composition of any one of E1-E92.
   E95. The vial of E94, wherein the vial is made of cyclic-olefin copolymer.
   E96. The vial of E94, wherein the vial is made of sterile high-density polyethylene (HDPE).
   E97. The vial of any one of E94-E96, wherein the vial has an in-place thermoplastic elastomer stopper.
   E98. A method of treating a disease comprising administering an effective amount of the pharmaceutical composition of any one of E1-E93 or E94-E97 to a human subject having a disease.
   E99. The method of E98, wherein the composition comprises about 3.0E+11 vg/mL to about 3.0E+13 vg/mL of rAAV vector.
   E100. The method of E98 or E99, wherein the pharmaceutical composition comprises about 4E+11 vg/mL, about 2E+12 vg/mL, about 1E+13 vg/mL or about 2E+13 vg/mL of the rAAV vector.
   E101. The method of any one of E98-E100, wherein the pharmaceutical composition is administered parenterally.
   E102. The method of any one of E98-E101, wherein the pharmaceutical composition is administered by intravenous injection, intraarterially injection, intracranial injection, intraperitoneal injection, portal vein injection, intramuscular injection, intrathecal administration, or subcutaneous injection. E103. The method of any one of E98-E102, wherein the pharmaceutical composition is administered intravenously.
   E104. The method of any one of E98-E103, wherein the pharmaceutical composition is administered at a dose of about 5E+11 vg/kg to about 1E+14 vg/kg.
   E105. The method of any one of E98-E104, wherein the pharmaceutical composition is administered at a dose of about 1E+12 vg/kg to about 1E+13 vg/kg.
   E106. The method of any one of E98-E105, wherein the disease is due to a deficiency or dysfunction of copper-transporting ATPase 2.
   E107. The method of any one of E98-E106, wherein the disease is Wilson disease.
   E108. A method of administering a pharmaceutical composition of any one of E1-E93 comprising a rAAV3B vector comprising an ATP7B transgene intravenously to a patient with Wilson disease at a dose of about 5E+11 vg/kg to about 1E+14 vg/kg.
   E109. A method of administering a pharmaceutical composition of any one of E1-E93 to a patient with Wilson disease at a dose of about 1E+12 vg/kg to about 1E+13 vg/kg.
   E110. A method of reducing hepatic copper in a human subject in need thereof, comprising administering intravenously to the human subject the pharmaceutical composition of any one of E1-E93, or the contents of the vial of any one of E94-E97.
   E111. The method of E110, wherein the human subject has Wilson disease.
   E112. A method of treating Wilson disease in a human subject in need thereof, comprising administering intravenously to the human subject a pharmaceutical composition of any one of E1-E93, or the contents of the vial of any one of E94-E97.
   E113. Use of the pharmaceutical compositions of any one of E1-E93 in the manufacture of a medicament for the treatment of disease.
   E114. The use of E113, wherein the disease is Wilson disease.
   E115. Use of the pharmaceutical composition of any one of E1-E93 in the manufacture of a medicament for treatment of a disease due to deficiency or dysregulation of copper-transporting ATPase 2.
   E116. The use of E115, wherein the disease is Wilson disease.
   E117. The use of any one of E113-E116, wherein the treatment is as defined in any one of E97 to E106.
   E118. The pharmaceutical composition of any one of E1-E93, for use in a method of treating a disease.
   E119. The pharmaceutical composition of any one of E1-E93 for use in a method of treating a human subject with a disease due to deficiency or dysregulation of copper-transporting ATPase 2.
   E120. The pharmaceutical composition, for use according to E118 or E119, wherein the disease is Wilson disease.
   E121. The pharmaceutical composition, for use according to any one of E118 to E120, wherein the method of treatment is as defined in any one of E98 to E107.
   E122. A kit comprising a pharmaceutical composition of any one of E1-E93, or the contents of the vial of any one of E94-E97, and instructions for use.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

The following Examples describe studies in which the inventors discovered a formulation for rAAV vectors that provides a substantial degree of stability over time, during freeze-thaw cycles and with agitation stress, while retaining quality attributes within acceptable levels and as compared to the rAAV vector prior to thermal and agitation stress testing. In some embodiments, the rAAV exhibiting a substantial degree of vector stability comprises at least one AAV3B capsid protein (e.g., VP3).

### Example 1: Thermal and Freeze-Thaw Stability of a rAAV3B Vector

The chemical and physical stability of a rAAV gene therapy vector for the treatment of Wilson disease was evaluated. The rAAV vector comprised an AAV3B capsid containing a single-stranded vector genome carrying a shortened version of the human ATP7B gene under the control of the liver-specific human α-1 antitrypsin (AAT) promoter, a polyA signal sequence and flanking ITRs. The nucleotide sequence of the transgene cassette comprised an ATP7B transgene encoding a polypeptide comprising the amino acid sequence of SEQ ID NO:1, an AAT promoter comprising the nucleic acid sequence of SEQ ID NO:3, a polyA signal sequence comprising the nucleic acid sequence of SEQ ID NO:4 and flanking AAV2 ITR sequences. The ATP7B gene encodes a copper transporting ATPase 2. Loss of function of the copper transporting ATPase 2 causes Wilson disease which is characterized by accumulation of copper in tissues, especially within the liver.

### 1. Materials and Methods

The rAAV vector was formulated as drug substance in 20 mM Tris, 100 mM MgCl₂, 4% (w/v) sucrose, 0.02% (w/v) poloxamer 188 at pH 7.6. The rAAV vector drug substance was prepared at 3 different concentrations (4E+11 vg/mL, 2E+12 vg/mL and 1E+13 vg/mL) and was stored in sterile high-density polyethylene (HDPE) vials (Fisher Scientific) at ≤−65° C.

Upon removal from frozen storage at ≤−65° C., the bulk drug substance was thawed at 2° C. to 8° C. and upon completion of thawing, was gently inverted 4 times. The 4E+11 vg/mL drug substance was prepared by diluting the thawed, bulk drug substance, which was at a concentration of 1E+13 vg/mL, 25× in the same formulation, by gently swirling several times. The 2E+12 vg/mL drug substance was prepared by diluting the thawed, bulk drug substance 5× in the same formulation, by gently swirling several times.

The diluted and neat drug substance samples were pulled into a syringe and filtered through a 0.22 μm polyvinyldene fluoride (PVDF) syringe filter (Millipore) into HDPE vials under a laminar flow hood. A fill volume of 1 mL in the 7 mL HDPE vial was used to represent the worst-case surface area to volume ratio.

Quality attributes of the drug substance formulated at a concentration of 1E+13 vg/mL were evaluated at time 0 and following 1) storage at <−65° C. for 7 days and 1 month; 2) storage at 2° C. to 8° C. for 1 day and 7 days; 3) storage at 25° C. for 8 hours and 24 hours; 4) 3 freeze/thaw (F/T) cycles consisting of storage at ≤−65° C. for 12+ hours followed by storage at 25° C. for 8 hours; and 5) agitation of the vials while on their sides for 24 hours at 300 rpms under controlled room temperature conditions. After each F/T cycle, the vials were inverted gently 4 times.

Quality attributes of the drug substance formulated at a concentration of 2E+12 vg/mL were evaluated at time 0 and following 1) storage at 2° C. to 8° C. for 5 days; and 2) storage at 25° C. for 24 hours. Quality attributes of the drug substance formulated at a concentration of 4E+11 vg/mL were evaluated at time 0 and following 1) storage at 2° C. to 8° C. for 1 day and 7 days; 2) storage at 25° C. for 8 hours and 24 hours; 3) 3 freeze/thaw (F/T) cycles consisting of storage at ≤−65° C. for 12+hours followed by storage at 25° C. for 8 hours; and 4) agitation of the vials while on their sides for 24 hours at 300 rpms under controlled room temperature conditions. After each F/T cycle, the vials were inverted gently 4 times.

Quality attributes included appearance (including color, clarity, visible particulate), percentage of capsids and percentage of each of VP1, VP2 and VP3 (measured by CGE, reducing), vg titer (vg/mL, measured by qPCR analysis of the transgene), infectious virus titer (IU/mL, measured by TCID50), sub-visible particles (measured by MFI), pH, viral particle (including full, empty and intermediate capsids) titer (vp/mL, measured by size exclusion chromatography (SEC)), % monomer (measured by SEC), % high molecular mass species (HMMS, measured by SEC and an indication of greater than one capsid), and A260/A280 as a measure of empty to full capsids. Aggregation was assessed by observation of visible particulate, measurement of sub-visible particles and % HMMS species. Osmolality as a measure of tonicity was measured at time 0.

### 2. Results

Results from the thermal stability studies of the drug substance formulated at 1E+13 vg/mL are shown in Table 1. All the quality attributes tested showed no significant change after 1 month at ≤65° C., 7 days at 2° C. to 8° C. and 24 hours at 25° C. All the quality attributes tested showed no significant change after 3 freeze-thaw cycles at ≤−65° C. for 12 or more hours followed by 8 hours at controlled room temperature of 25° C. All the quality attributes tested showed no significant change and were within method variability. The osmolality of the drug substance was 436 mOsm/kg H₂O at time 0. The drug substance had a density of 1.025 g/cm³, a viscosity of 1.193 mPa and a conductivity of 16.94 mS/cm at time 0.

TABLE 1
Quality attributes of drug substance at 1E+13 vg/mL under thermal stability testing.

| | | | | | | | | ≤−65° C./ | |
|---|---|---|---|---|---|---|---|---|---|
| Quality | | ≤−65° C. | | 2° C. to 8° C. | | 25° C. | | 25° C. | 300 rpms |
| Attribute | Time 0 | 7 days | 1 month | 1 day | 7 days | 8 hours | 24 hours | 3 cycles | 24 hours |
| ≤Color | ≤B9 | NT | ≤B9 | NT | ≤B9 | NT | ≤B9 | ≤B9 | ≤B9 |
| ≤Clarity | ≤Ref I | NT | ≤Ref I | NT | ≤Ref I | NT | ≤Ref I | ≤Ref I | ≤Ref I |
| Visible | 1-5 | NT | EFVP | NT | EFVP | NT | EFVP | EFVP | EFVP |
| particles | particles* | | | | | | | | |
| % capsid | 99.3 | 99.0 | 99.4 | 99.3 | 98.9 | 99.5 | 99.0 | 98.9 | 99.2 |
| % other | 0.7 | 1.0 | 0.6 | 0.7 | 1.1 | 0.5 | 1.0 | 1.1 | 0.8 |
| % VP1 | 11.7 | 11.8 | 11.8 | 11.8 | 12.1 | 12.1 | 12.1 | 11.6 | 11.8 |
| % VP2 | 18.5 | 18.4 | 18.3 | 18.3 | 18.9 | 17.2 | 18.7 | 18.3 | 19.4 |
| % VP3 | 69.9 | 69.8 | 69.9 | 69.8 | 69.0 | 70.7 | 69.2 | 70.1 | 68.8 |
| titer (vg/mL) | 1.56E+13 | 2.22E+13 | 1.75E+13 | 1.95E+13 | 1.83E+13 | 1.68E+13 | 1.86E+13 | 2.13E+13 | 1.42E+13 |
| titer (IU/mL) | 5.22E+11 | NT | 1.65E+12 | NT | NT | NT | 2.42E+11 | NT | NT |
| sub-visible | 125 | NT | 37 | NT | 12 | NT | 50 | 0 | 150 |
| particles | | | | | | | | | |
| (≥10 μm) | | | | | | | | | |
| sub-visible | 25 | NT | 25 | NT | 0 | NT | 0 | 0 | 25 |
| particles | | | | | | | | | |
| (≥25 μm) | | | | | | | | | |
| pH | 7.6 | 7.5 | 7.7 | 7.5 | 7.5 | 7.6 | 7.6 | 7.6 | 7.6 |
| viral particle | 1.04E+14 | 9.19E+13 | 9.99E+13 | 9.16E+13 | 9.17E+13 | 9.13E+13 | 9.20E+13 | 9.13E+13 | 9.87E+13 |
| titer (vp/mL) | | | | | | | | | |
| % monomer | 99.4 | 99.2 | 99.0 | 99.2 | 99.1 | 99.2 | 99.4 | 99.2 | 98.6 |
| % HMMS | NMT 1.0 | NMT 1.0 | NMT 1.0 | NMT 1.0 | NMT 1.0 | NMT 1.0 | NMT 1.0 | NMT 1.0 | 1.4 |
| A260/A280 | 1.08 | 1.10 | 1.06 | 1.09 | 1.10 | 1.09 | 1.09 | 1.10 | 1.08 |

*particles identified as environmental white strands; ≤Ref I: less opalescent or equal to reference standard I; NT: not tested; NMT: not more than; EFVP: essentially free of visible particles; B9: clear, colorless.

Results from thermal stability studies of the drug substance formulated at 2E+12 vg/mL are shown in Table 2. All quality attributes tested showed no significant change after 5 days at 2° C. to 8° C. and 24 hours at 25° C. The osmolality of the drug substance was 436 mOsm/kg at time 0.

TABLE 2
Quality attributes of drug substance at 2E+12
vg/mL under thermal stability testing.

| | | 2° C. to 8° C. | 25° C. |
|---|---|---|---|
| Quality Attribute | Time 0 | 5 days | 8 hours |
| ≤Color | ≤B9 | ≤B9 | ≤B9 |
| ≤Clarity | ≤Ref I | ≤Ref I | ≤Ref I |
| visible particles | EFVP | EFVP | EFVP |
| % capsid | 99.8 | 99.8 | 99.8 |
| % other | 0.2 | 0.2 | 0.2 |
| % VP1 | 10.6 | 10.8 | 10.6 |
| % VP2 | 17.3 | 17.8 | 18.3 |
| % VP3 | 72.1 | 71.5 | 71.1 |
| titer (vg/mL) | 3.28E+12 | 3.60E+12 | 3.20E+12 |
| titer (IU/mL) | 1.02E+11 | NT | 5.22E+10 |
| sub-visible particles (≥10 μm) | 162 | 50 | 25 |
| sub-visible particles (≥25 μm) | 25 | 0 | 12 |
| pH | 7.5 | 7.6 | 7.6 |
| viral particle titer (vp/mL) | 1.94E+13 | 1.94E+13 | 1.89E+13 |
| % monomer | 99.0 | 99.0 | 98.9 |
| % HMMS | NMT 1.0 | NMT 1.0 | 1.1 |
| A260/A280 | 1.06 | 1.06 | 1.07 |

≤Ref I: less opalescent or equal to reference standard I; NT: not tested; NMT: not more than; EFVP: essentially free of visible particles; B9: clear, colorless.

Results from thermal stability testing of the drug substance formulated at 4E+11 vg/mL are shown in Table 3. Appearance, pH, viral genome titer, infectious titer, subvisible particles, viral particle titer and capsid ratio showed no significant changes after 7 days at 2° C. to 8° C. and 24 hours at 25° C. There was a slight increase in aggregation after 24 hours at controlled room temperature of 25° C. The concentration of the drug substance was at the lowest level of acceptability for purity (measured by CGE method). All quality attributes tested showed no significant change after 3 freeze-cycles at ≤−65° C. for 12 or more hours followed by 8 hours at controlled room temperature of 25° C. There was an increase in aggregation after undergoing an extreme shear stress of 24 hours at 300 rpms. All other quality attributes tested did not have any significant changes and all were within method variability. The osmolality of the drug substance was 436 mgOsm/kg at time 0.

TABLE 3
Quality attributes of drug substance at 4E+11 vg/mL under thermal stability testing.

| | | | | | | ≤−65° C./ | |
|---|---|---|---|---|---|---|---|
| Quality | | 2° C. to 8° C. | | 25° C. | | 25° C. | 300 rpms |
| Attribute | Time 0 | 1 day | 7 days | 8 hours | 24 hours | 3 cycles | 24 hours |
| ≤Color | ≤B9 | NT | ≤B9 | NT | ≤B9 | ≤B9 | ≤B9 |
| ≤Clarity | ≤Ref I | NT | ≤Ref I | NT | ≤Ref I | ≤Ref I | ≤Ref I |
| visible particles | EFVP | NT | EFVP | NT | EFVP | EFVP | EFVP |
| % VP1 | 8.3 | 10.2 | 10.7 | 9.8 | 8.4 | 10.3 | 9.7 |
| % VP2 | 17.9 | 10.6 | 11.9 | 8.7 | 12.2 | 11.8 | 17.5 |
| % VP3 | 73.8 | 79.2 | 77.5 | 81.4 | 79.4 | 77.9 | 72.7 |
| Titer (vg/mL) | 6.02E+11 | 8.41E+11 | 9.75E+11 | 7.56E+11 | 7.99E+11 | 7.70E+11 | 5.63E+11 |
| Titer (IU/mL) | 2.00E+10 | NT | NT | NT | 1.65E+10 | NT | NT |
| Sub-visible | 75 | NT | 25 | NT | 25 | 12 | 62 |
| particles (≥10 μm) | | | | | | | |
| Sub-visible | 12 | NT | 0 | NT | 0 | 0 | 25 |
| particles (≥25 μm) | | | | | | | |
| pH | 7.4 | 7.5 | 7.4 | 7.5 | 7.6 | 7.5 | 7.6 |
| Viral particle titer | 2.91E+12 | 3.44E+12 | 2.95E+12 | 3.17E+12 | 3.37E+12 | 3.12E+12 | 2.88E+12 |
| (vp/mL) | | | | | | | |
| % monomer | 99.1 | 98.3 | 99.2 | 98.8 | 97.8 | 99.3 | 93.7 |
| % HMMS | NMT 1.0 | 1.7 | NMT 1.0 | 1.2 | 2.2 | NMT 1.0 | 6.3 |
| A260/A280 | 1.09 | 1.10 | 1.09 | 1.10 | 1.10 | 1.08 | 1.07 |

≤Ref I: less opalescent or equal to reference standard I; NT: not tested; NMT: not more than; EFVP: essentially free of visible particles.

This study demonstrated that the rAAV3B vector at 4E+11 vg/mL to 1E+13 vg/mL in 20 mM Tris, 100 mM MgCl₂, 4.0% (w/v) sucrose and 0.02% (w/v) poloxamer 188 at pH 7.6 was stable for up to 1 month at ≤−65° C., for 7 days at 2° C. to 8° C. and for 24 hours at 25° C. Additionally, the formulation was stable when exposed to up to 3 freeze-thaw cycles between ≤−65° C. and 25° C.

### Example 2: Freeze/Thaw Cycling and Agitation Tests

### 1. Source of rAAV3B Vector and Preparation of Formulation

Multiple lots of rAAV3B vector, comprising a copper transporting ATPase 2 transgene (ATP7B) as described in Example 1, were used in these studies. All lots underwent ultrafiltration/diafiltration to achieve a target concentration of 1.1E+13 vg/mL in 20 mM Tris, 100 mM MgCl₂ at pH 7.6. The material was formulated by spiking in excipient buffer to achieve a final concentration of 1E+13 vg/mL rAAV3B vector in 20 mM Tris, 100 mM MgCl₂, 4% (w/v) sucrose, 0.02% (w/v) poloxamer 188 at pH 7.6. Lower concentrations were prepared by diluting with formulation buffer or n-saline.

### 2. Long Term Storage, Accelerated Stress Method and Dosing Solution Stability

Filled vials were stored in controlled stability chambers to maintain constant temperature during storage testing. The chambers were maintained at −90° C. to −60° C. (reported as −70° C. in the results), 2° C. to 8° C. (reported as 5° C. in the results), and 23° C. to 27° C. with 65% relative humidity (RH). Vials were stored upright for 1 month, 3 months and 6 months for the long-term storage studies.

The accelerated stress method used uncontrolled freeze-thaw cycles. The uncontrolled freeze-thaw was performed by freezing the vials in a −90°° C. to −60° C. chamber followed by thawing at 2° C. to 8° C.

Agitation stress was evaluated by benchtop shaking of the vials on an orbital shaker at 300 rpms for 24 hours.

### 3. Experimental Methods

Visual inspections were performed by comparing color and opalescence standards as well as inspecting for visible particulates. Viral particle content, empty/full capsid ratio, viral purity and aggregation were monitored by size exclusion-high performance liquid chromatography (SE-HPLC). Transgene content was monitored by quantitative PCR (qPCR). Infectivity was monitored by TCID50 for activity. Capsid purity was monitored by reduced capillary gel CGE (rCGE). Sub-visible particle counts were assessed using mean fluorescent intensity (MFI). pH was also monitored.

### 4. Results

The long term and accelerated stability studies were run to verify the suitability of the formulation. The stability study investigated the long term and accelerated stability of the rAAV3B vector in the formulation comprising 20 mM Tris, 100 mM MgCl₂, 4% sucrose, 0.02% poloxamer 188 at pH 7.6 in a cyclic olefin primary container vial. FIG. 1-10 illustrate the stability of the rAAV3B vector in the primary container at the long term intended storage condition of −70° C. as well as under accelerated storage conditions of −20° C. and 5° C. for up to 6 months. All timepoints were assessed for appearance and were clear, colorless and essentially free from visible particulates.

Transgene titer (vg/mL) of the formulation comprising the rAAV3B vector stored at −70° C., −20° C. and 5° C. was measured by qPCR at time 0, 1 month, 3 months and 6 months. No significant difference in transgene titer was observed between time points or between storage conditions (FIG. 1).

Viral particle titer (vp/mL) of the formulation comprising the rAAV3B vector stored at −70° C., −20° C. and 5° C. was measured by SE-HPLC at time 0, 1 month, 3 months and 6 months. No significant difference in viral particle titer was observed between time points or between storage conditions (FIG. 2).

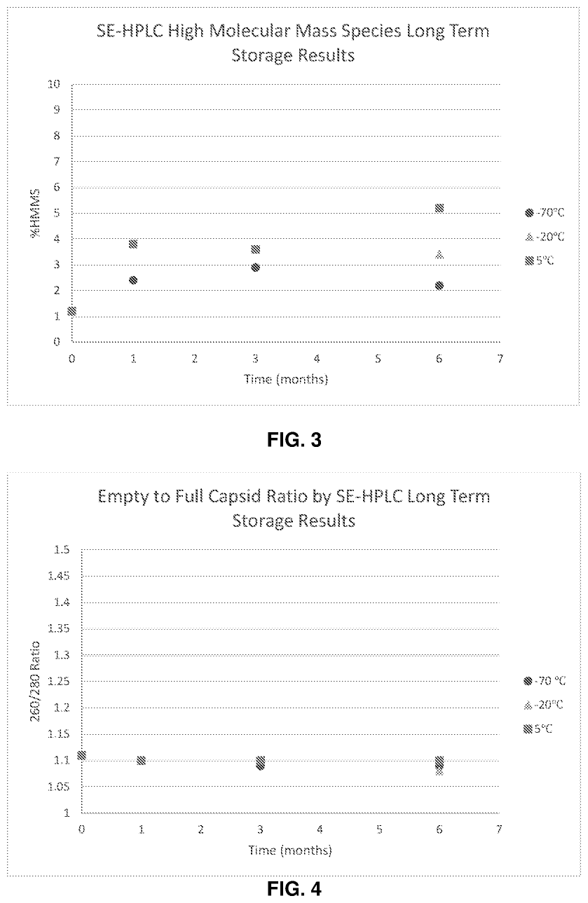

The percentage of HMMS of the formulation comprising the rAAV3B vector stored at −70° C., −20° C. and 5° C. was measured by SE-HPLC at time 0, 1 month, 3 months and 6 months. No significant difference in percentage of HMMS was observed between time points or between storage conditions (FIG. 3).

The ratio of empty to full capsids in the formulation comprising the rAAV3B vector stored at −70° C., −20° C. and 5° C. was measured at A260/A280 by SE-HPLC at time 0, 1 month, 3 months and 6 months. No significant difference in ratio of empty to full capsids was observed between time points or between storage conditions (FIG. 4).

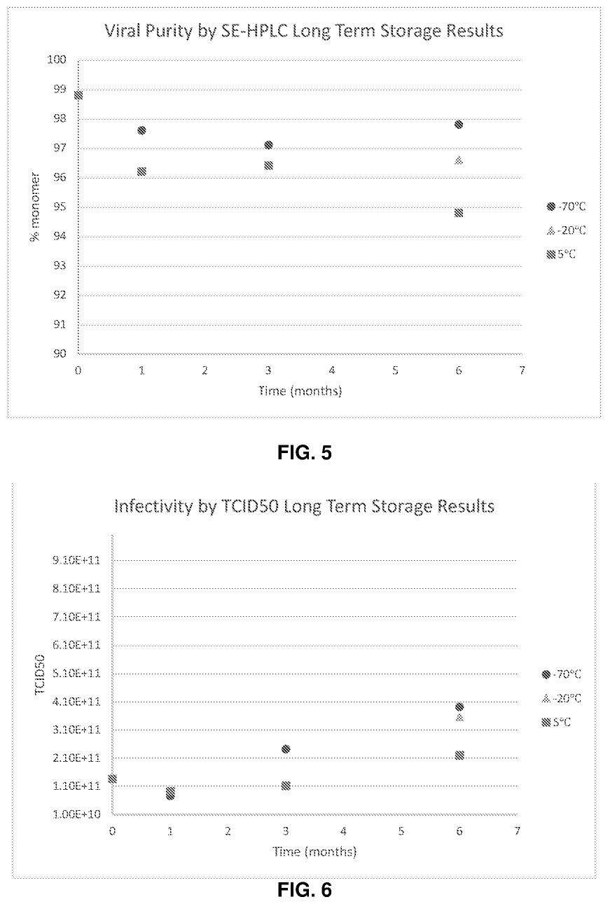

The viral purity (% monomer) of the formulation comprising the rAAV3B vector stored at −70° C., −20° C. and 5° C. was measured by SE-HPLC at time 0, 1 month, 3 months and 6 months. A slight decrease in purity was observed for the compositions stored at 5° C. by 6 months but did not exceed a predetermined acceptability threshold of 85% (FIG. 5).

The vector infectivity of the formulation comprising the rAAV3B vector stored at −70° C., −20° C. and 5° C. as measured by Median Tissue Culture Infectious Dose (TCID50) at time 0, 1 month, 3 months and 6 months. The TCID50 increased under all storage conditions by 6 months and thus did not fall below a minimum level of acceptability (FIG. 6).

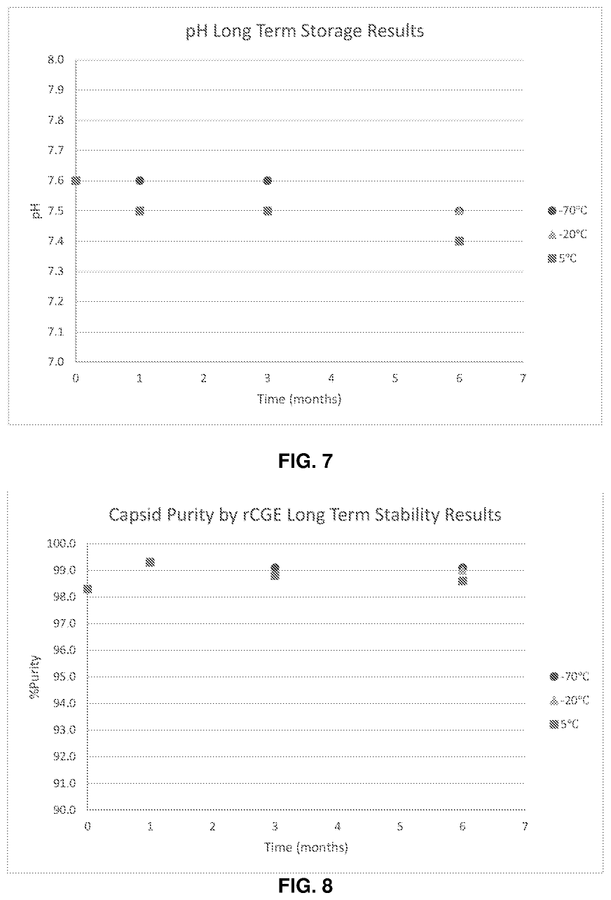

The pH of the formulation comprising the rAAV3B vector stored at −70° C., −20° C. and 5° C. was measured at time 0, 1 month, 3 months and 6 months. No significant difference in pH was observed between time points or between storage conditions (FIG. 7).

The percent purity of the capsids in the formulation comprising the rAAV3B vector stored at −70° C., −20° C. and 5° C. was measured by capillary gel electrophoresis, reducing (rCGE) at time 0, 1 month, 3 months and 6 months. No significant difference in the capsid percent purity was observed between time points or between storage conditions (FIG. 8).

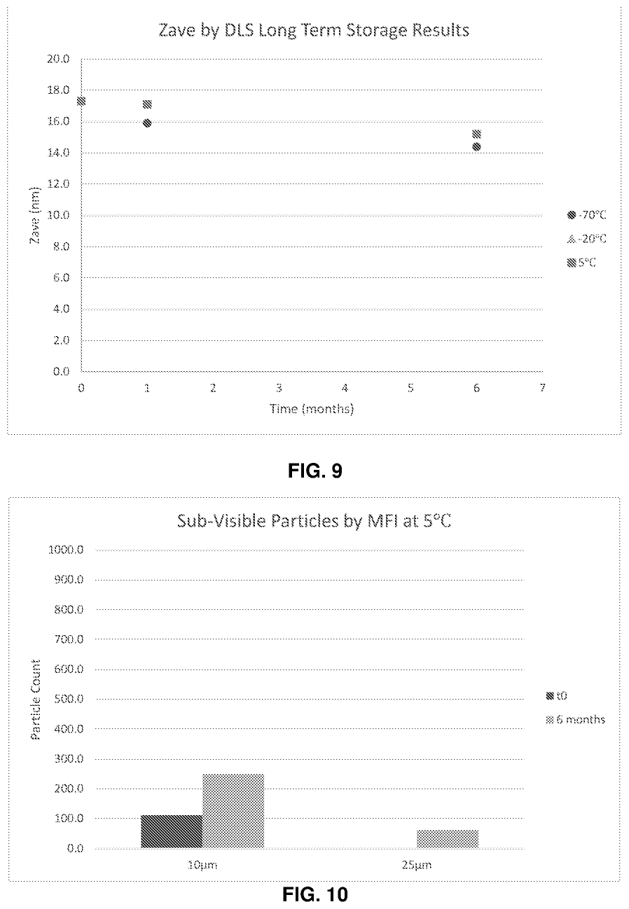

The Z-average (nm) of the formulation comprising the rAAV3B vector stored at −70° C., −20°° C. and 5° C. was measured by dynamic light scattering (DLS) at time 0, 1 month and 6 months. A Z-average is a measure the level of aggregation of rAAV capsids present in a solution. No significant difference in the Z-average was observed between time points or between storage conditions (FIG. 9).

The number of sub-visible particles, in the range of 10 μm and 25 μm, present in the formulation comprising the rAAV vector and stored at −70° C., −20° C. and 5° C. was measured by mean fluorescent intensity (MFI) at time 0 and 6 months. There was a slight increase in the number of particles in the 10 μm range at 6 months as compared to the number at time 0. Also, while no particles in the 25 μm range were observed at time 0, less than 100 particles in this range were observed at time 6 months (FIG. 10). Neither of the increases in sub-visible particles observed at 6 months exceeded a pre-determined threshold of acceptability of 6000 particles at 10 μm and 600 particles of 25 μm.

The formulation comprising the rAAV3B vector was tested by an accelerated stress method of 5 uncontrolled freeze-thaw cycles, each consisting of freezing at 90° C. to 60° C. for 12 hours and thawing at 2° C. to 8° C. for 8 hours in the original vial. At time 0, and following the 5 freeze thaw cycles, the vials were assessed for appearance and were observed to be clear, colorless and essentially free from visible particulates.

There was no significant difference in the quality attributes measured between the formulation comprising the rAAV3B vector at time 0 and following the 5 freeze-thaw cycles.

TABLE 4
rAAV vector formulation quality attributes
following 5 freeze-thaw cycles.

| | | after 5 freeze/thaw |
|---|---|---|
| Quality Attribute | time 0 | cycles |
| % capsid purity by rCGE | 98.8 | 98.6 |
| Z-average (nm) by DLS | 15 | 15.1 |
| % HMMS by SE-HPLC | 3.6 | 2.5 |
| viral purity (% monomer) by SE-HPLC | 96.4 | 97.5 |
| empty to full capsid ratio | 0.91 | 0.9 |
| by A260/A280, SE-HPLC | | |
| viral particle titer by SE-HPLC (vp/mL) | 8.20E+13 | 8.02E+13 |
| vg titer by transgene qPCR (vg/mL) | 1.18E+13 | 1.15E+13 |
| infectivity by TCID50 (IU/mL) | 6.97E+11 | 2.42E+11 |

Agitation stress was assessed after the vials containing the rAAV vector formulation were placed horizontally on an orbital shaker at room temperature and agitated for 24 hours at 300 rpms. Both time points were assessed for appearance and were clear, colorless and essentially free from visible particulates.

TABLE 5
rAAV vector formulation attributes following agitation stress

| Attributes | Time 0 | Time 24 hours |
|---|---|---|
| % capsid purity by rCGE | 98.8 | 98.6 |
| Z-average (nm) by DLS | 15 | 15.3 |
| % HMMS by SE-HPLC | 3.6 | 3.2 |
| viral purity (% monomer) by SE-HPLC | 96.4 | 96.8 |
| empty to full capsid ratio by A260/A280, | 0.91 | 0.92 |
| SE-HPLC | | |
| viral particle titer by SE-HPLC (vp/mL) | 8.20E+13 | 8.11E+13 |
| vg titer by transgene qPCR (vg/mL) | 1.18E+13 | 1.29E+13 |
| infectivity by TCID50 (IU/mL) | 6.97E+11 | 2.00E+11 |

Long term and accelerated storage, combined with additional stress of freeze thaw cycling and agitation, indicates that rAAV vector in the formulation studied was stable with minimal to no observed degradation.

### Example 3: Assessment of Susceptibility of AAV3B Capsids to Aggregation in the Presence or Absence of Simple Salts

A rAAV3B vector, comprising a copper transporting ATPase 2 transgene (ATP7B) as described in Example 1, was used in these studies. Relatively full (F) and relatively empty (E) rAAV3B vector, were buffer exchanged using 50 kDa membrane to a 1) a test formulation (20 mM Tris, 100 mM MgCl₂, 4% w/v sucrose, 0.02% poloxamer 188, pH 7.6), 2) PBS with 150 mM NaCl, 3) 10 mM Tris pH 7.4, 4) Tris buffer spiked with NaCl, 5) Tris buffer spiked with MgCl₂ or 6) Tris buffer spiked with CaCl₂.

Dynamic light scattering (DLS), Size Exclusion Chromatography (SEC), visual appearance, Mean Fluorescent Intensity (MFI), optical microscopy and NTA (Nanoparticle Tracking Analysis) data were collected for all samples and analyzed to determine the effect of salt contribution on suppression of vector aggregation during buffer exchange and following 2 weeks storage at 5° C.

### 1. Materials

rAAV3B vector drug substance was supplied in 30 mM sodium citrate, 85 mM Tris, 200 mM sodium acetate, 0.0085% P188 at pH 7.6. A sample of relatively empty (E) rAAV3B vector had a concentration of 6.43E+13 viral particles (VP)/mL with a R=0.61. A sample of relatively Full (F) rAAV3B had a concentration of 5.89E+13 VP/mL with a R=0.86.

### 2.1 Methods-Buffer Exchange Screening

Relatively empty and relatively full rAAV3B vector samples were exchanged from 30 mM sodium citrate, 85 mM Tris, 200 mM sodium acetate, 0.0085% P188 at pH 7.6 to each of the test formulation, 1× PBS and 20 mM Tris, pH 7.4 (no salt) through 50 kDa membranes and rotor centrifugation. Membranes were washed with water and 2× buffer. The exchange was performed at 3000 RCF with 3 volumes of each of the buffers. After the exchange, each sample was spiked with 20 μL of 1% P188. The final sample volume was normalized to 100 μL.

50 μL of each sample was filtered through a 0.8 μm gold filter and rinsed with water. Samples were evaluated by optical microscopy and Fourier-transform infrared spectroscopy-attenuated total reflectance (FTIR-ATR). The relatively full sample in Tris had haziness, which was less obvious in the relatively empty sample in Tris. The relatively empty sample in PBS had ring-like haziness while the relatively full sample in PBS had bright particulates. The relatively empty and relatively full samples in the test formulation were relatively clean. The rAAV3B vector samples in Tris were analyzed by FTIR-microscopy in reflectance mode.

### 2.2 Methods—2 Week Stability at 5° C.

40 mL of each buffer was prepared and spiked with 80 μL of 0.1% P188. Membranes were washed with water and 2× buffer. Relatively empty and relatively full rAAV3B vector samples were exchanged from 30 mM sodium citrate, 85 mM Tris, 200 mM sodium acetate, 0.0085% P188 at pH 7.6 at 3000 RCF with 3 volumes of each buffer shown in Table 6. The final volume of each sample was normalized to 2000 μL. Samples were sterile filtered using 0.22 μm PVDF membranes.

TABLE 6
Sample Treatment

| Sample | Sample | | Membrane | Centrifugation |
|---|---|---|---|---|
| Type | Name | Buffer | Size | Speed, RCF |
| rAAV3B | test formulation | 20 mM Tris, 100 mM MgCl₂, 4% w/v | 50 KDa | 3,000 |
| Fract.1 | | sucrose, 0.02% poloxamer 188, pH | | |
| (R = 0.61) | | 7.6 | | |
| Relatively | PBS | 1X PBS (includes 150 mM NaCl) | 50 KDa | 3,000 |
| Empty | Tris | 10 mM Tris pH 7.4 0.02% P188 | 50 KDa | 3,000 |
| Lot 1 | 12 mM NaCl | 10 mM Tris pH 7.4 0.02% P188 | 50 KDa | 3,000 |
| | | 12.5 mM NaCl | | |
| | 12 mM MgCl2 | 10 mM Tris pH 7.4 0.02% P188 | 50 KDa | 3,000 |
| | | 12.5 mM MgCl2 | | |
| | 12 mM CaCl2 | 10 mM Tris pH 7.4 0.02% P188 | 50 KDa | 3,000 |
| | | 12.5 mM CaCl2 | | |
| | 100 mM NaCl | 10 mM Tris pH 7.4 0.02% P188 0.1M | 50 KDa | 3,000 |
| | | NaCl | | |
| | 100 mM MgCl2 | 10 mM Tris pH 7.4 0.02% P188 0.1M | 50 KDa | 3,000 |
| | | MgCl2 | | |
| | 100 mM CaCl2 | 10 mM Tris pH 7.4 0.02% P188 0.1M | 50 KDa | 3,000 |
| | | CaCl2 | | |
| | 2000 mM NaCl | 10 mM Tris pH 7.4 0.02% P188 2M | 50 KDa | 3,000 |
| | | NaCl | | |
| | 2000 mM | 10 mM Tris pH 7.4 0.02% P188 2M | 50 KDa | 3,000 |
| | MgCl2 | MgCl2 | | |
| rAAV3B | test formulation | 20 mM Tris, 100 mM MgCl₂, 4% w/v | 50 KDa | 3,000 |
| Fract.4 | | sucrose, 0.02% poloxamer 188, pH | | |
| (R = 0.86) | | 7.6 | | |
| Relatively | PBS | 1X PBS (includes 150 mM NaCl) | 50 KDa | 3,000 |
| Full | Tris | 10 mM Tris pH 7.4 0.02% P188 | 50 KDa | 3,000 |
| | 12 mM NaCl | 10 mM Tris pH 7.4 0.02% P188 | 50 KDa | 3,000 |
| | | 12.5 mM NaCl | | |
| | 12 mM MgCl2 | 10 mM Tris pH 7.4 0.02% P188 | 50 KDa | 3,000 |
| | | 12.5 mM MgCl2 | | |
| | 12 mM CaCl2 | 10 mM Tris pH 7.4 0.02% P188 | 50 KDa | 3,000 |
| | | 12.5 mM CaCl2 | | |
| | 100 mM NaCl | 10 mM Tris pH 7.4 0.02% P188 0.1M | 50 KDa | 3,000 |
| | | NaCl | | |
| | 100 mM MgCl2 | 10 mM Tris pH 7.4 0.02% P188 0.1M | 50 KDa | 3,000 |
| | | MgCl2 | | |
| | 100 mM CaCl2 | 10 mM Tris pH 7.4 0.02% P188 0.1M | 50 KDa | 3,000 |
| | | CaCl2 | | |
| | 2000 mM NaCl | 10 mM Tris pH 7.4 0.02% P188 2M | 50 KDa | 3,000 |
| | | NaCl | | |
| | 2000 mM | 10 mM Tris pH 7.4 0.02% P188 2M | 50 KDa | 3,000 |
| | MgCl₂ | MgCl₂ | | |

### 2.3 Analytical Methods

Size Exclusion Chromatography: was used to calculate viral particle concentration (VP/mL), % high molecular mass species (HMMS), AAV monomer purity at 214 nm and ratio of AAV monomer peak at 260 nm/280 nm. Nucleic acids are known to have absorbance maxima at 260 nm while proteins are expected to have absorbance maxima around 280 nm. A 260 nm/280 nm ratio was reported to estimate relative changes in full vs empty capsids in a solution (see Sommer et al. (2003) Molecular Therapy 7(1):122-128). A decreased ratio indicated loss of DNA-filled capsids (e.g., full capsids) and an increased ratio indicated loss of empty capsids. Injection volume was varied to ensure that the sample area fell within the limits of the standard curve.

Dynamic Light Scattering: DynaPro plate reader was used to measure hydrodynamic radius, polydispersity and normalized intensity of all samples. In addition, % mass and radius of the smallest peak was estimated using Dynamics software and reported. Measurements were performed in duplicate and averages reported. Parameters used in this assay are derived at least in part from Stetefeld et al. (2016) Biophys. Rev. 8(4):409-427.

Nanosight (NTA): NTA data was collected using a Nanosight instrument. A camera was set to level 12 and is sensitive to several user-selected parameters. Parameters used in this assay are derived at least in part from Hubert et al. (2020) J. Pharm. Sci. 109:830-844.

In this example, the detection threshold was set to a conservative value of 20 in order to monitor particles in 50-500 nm range. Particles below 50 nm are not likely to be detected with the threshold value of 20 nm. Based on visual data analysis there was a correlation between the amount of particles <100 nm and HMMS. Therefore counts <100 nm were reported. Also counts >=100 nm and >=500nm were reported to examine distribution trends and to compare them to MFI. Both DLS and NTA used Stokes-Einstein equation to convert particle diffusion coefficient to the particle diameter. Viscosity was used in the denominator of the equation. Therefore, data was normalized for viscosity, which was measured and reported in this example.

Mean fluorescent intensity (MFI): was collected and counts/mL for the following size bins were reported: >=1 μm, >=2 μm, >=5 μm, >=8 μm, >=10 μm, >=25 μm, >=1 μm was compared to Nanosight >=500 μm.

Intrinsic Differential Scanning calorimetry (DSF) and Static Light Scattering (SLS): were monitored by Nanotemper Prometheus instrument at 20° C. to 95° C. at 1 C/min. Integration was performed automatically in the software.

Dye-based Differential Scanning calorimetry (DSF): was performed using a Biorad CFX96 thermo-cycler to monitor dye-specific fluorescence bands after spiking samples with DNA-specific dye SYBR Gold and protein-specific dye SYPRO Orange. Dye-based DNA detection methods are known to resolve multiple peaks during heating of AAV materials. For simplicity of data interpretation, initial fluorescence and first peak onset were reported. Data for various samples loaded on the same run were also compared qualitatively. Integration was performed in Lumetics LINK software.

### 3. Results

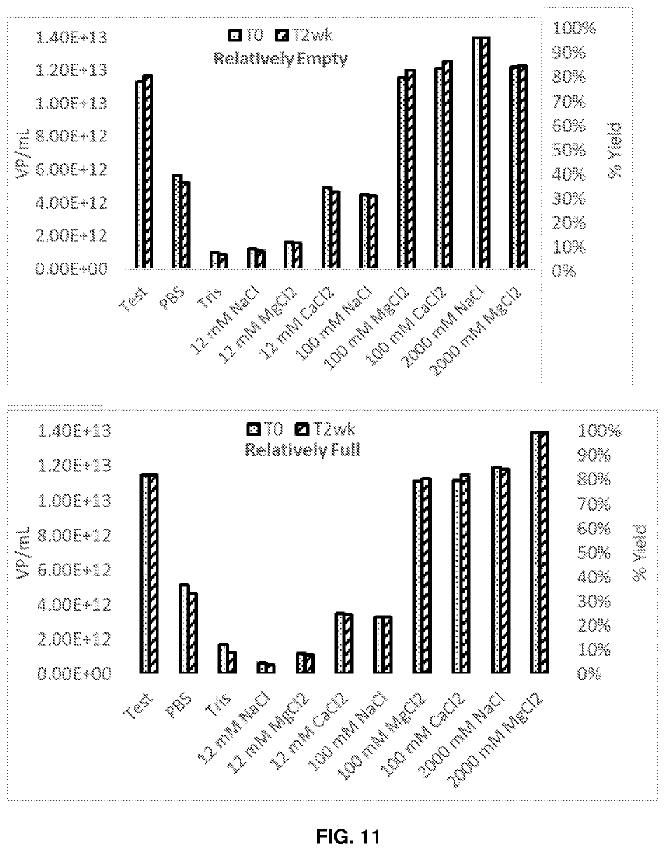

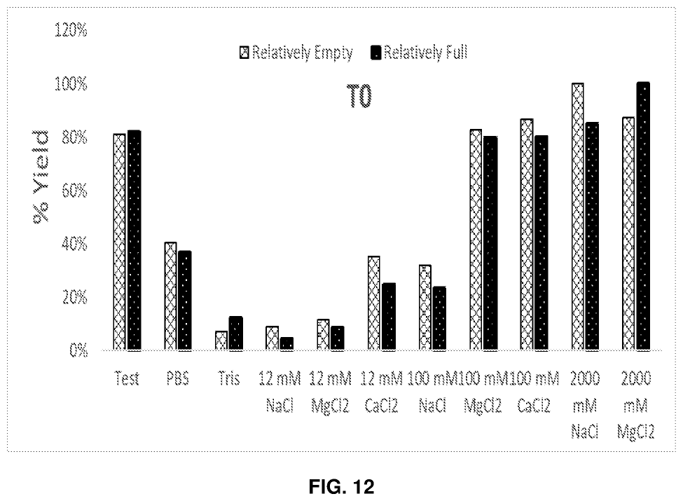

Viral capsid titer: rAAV titer (VP/mL) as measured by SEC-HPLC is shown in Table 7 (time 0), Table 8 (time 2 weeks at 5° C.) and in FIG. 11. AAV monomer viral capsid titer recovery was very low (below 12%) in those samples in Tris, 12 mM NaCl, and 12 mM MgCl₂ buffers. Viral capsid titer recovery was moderate (above 25%, but below 40%) in those samples in PBS, 12 mM CaCl₂ and 100 mM NaCl. Good recoveries, about 80%, were observed in samples in the test formulation, 100 mM MgCl₂, 100 mM CaCl₂, 2 M NaCl and 2M MgCl₂ buffers. A sample was not prepared in 2 M CaCl₂ due to concern about calcium precipitation in the phosphate mobile phase during chromatography.

Titer decrease was observed due to centrifugation shear when performing the buffer exchanges in all samples. As shown in Table 6, the viral capsid titer in the starting materials was 5.89E+13 VP/mL (relatively full) and 6.43E+13 VP/mL (relatively empty) and decreased to less than 1.4E+13 VP/mL. In contrast, no significant decrease in viral particles was observed after 2 weeks at 5° C. in any of the tested samples (FIG. 11).

Recoveries were slightly better for the relatively empty capsids as compared to the relatively full capsids, except for those samples in the test formulation, Tris and 2 M MgCl₂ (FIG. 12). The test formulation was the most robust as it showed the least amount of variation in viral titer between the relatively full capsid sample and the relatively empty capsid sample after the buffer exchange at T0. The ability to maintain consistent stability of both full and empty rAAV vectors is a beneficial characteristic of the AAV vector test formulation.

These data also demonstrate that a concentration of salt (e.g., a divalent cation, e.g., MgCl₂, CaCl₂) that is 100 mM or greater is necessary to maintain viral particle titer and limit aggregation of the capsids. While VP/mL for both relatively empty and relatively full capsid samples in a 100 mM MgCl₂ or a 100 mM CaCl₂ buffer at time 0 and time 2 weeks were similar, formulations comprising MgCl₂ are preferred over CaCl₂ for several reasons. Calcium ions are known to interfere with analytical methods by forming particles (e.g., when an assay reagent contains phosphate) making analysis of quality attributes difficult. Also, calcium can contribute to the formation of particles in a drug product and/or drug substance over long term storage. Hence, MgCl₂ is the preferred divalent cation for buffer formulations.

HMMS, DLS, NTA and MFI: Since samples were filtered after the exchange, it is possible that aggregates of about 0.2 μm were filtered out at T0. After 2 weeks at 5° C., a significant amount of particulate matter was detected in the Tris, 12 mM NaCl and 12 mM MgCl₂ samples by MFI (Table 9). Atypically high counts were detected by NTA in the PBS sample (Table 9).

Significant titer loss and subvisible particulate formation did not correlate an increase in % HMMS. It was also noted that HMMS did not vary linearly or proportionally with AAV monomer peak loss. For example, the AAV monomer peak from the 12 mM MgCl₂ relatively empty sample, as measured at 214 nm at T0, was considerably smaller (less than 500K AU) than the AAV monomer peak from the 12 mM CaCl₂ relatively empty sample (greater than 1300K AU), yet the HMMS peak area, as measured at 214 nm, remained nearly the same for these samples (i.e., 2,799 AU and 2,292AU, respectively). Thus, the % HMMS in the MgCl₂ sample was calculated to be three fold higher than the % HMMS in the CaCl₂ sample (0.6% v 0.2%). This phenomenon was more pronounced in the PBS relatively full sample at T0 (i.e., HMMS area of about 11K AU and AAV monomer area of 1,287K AU) as compared to the Tris relatively full sample at T0 (i.e., HMMS area of about 10K AU and AAV monomer area of about 500K AU). Thus, while the HMMS areas were not significantly different (about 11K AU vs. about 10K AU, respectively, the calculated % HMMS was (0.9% v. 2.2%, respectively) (Table 10).

DLS results that showed a significant increase in particle radius (an indication of aggregation) correlated with the samples with the greatest titer loss (Table 7 and 8). This was observed at T0 (i.e., immediately after filtration) and after 2 weeks. For instance, the average particle radius of the 12 mM NaCl sample by DLS was 242.9 nm and the AAV titer was 5.13E+11 VP/mL (relatively full at T0). In contrast, the average particle radius of the test formulation by DLS was 20.1 nm and the AAV titer was 9.26E+12 VP/mL (relatively full sample at T0). At time 2 weeks, the average particle radius of the 12 mM NaCl sample by DLS was 242.9 nm and the AAV titer was 5.13E+11 VP/mL (relatively full at T2wk). In contrast, the average particle radius of the test formulation by DLS was 14.3 nm and the AAV titer was 9.45E+12 VP/mL (relatively full sample at T2wk). Thus, DLS measurements can be highly impacted by reduction in the AAV titer.

High levels of AAV3B aggregation in Tris, 12 mM NaCl and 12 mM MgCl₂ was confirmed by MFI (Table 9). For instance, a relatively full sample in Tris (T2wks) had an MFI of 469,090 counts/mL at 1 μm whereas a relatively full sample in test formulation (T2wks) with little aggregation had an MFI of 6,708 counts/mL at 1 μm. AAV3B aggregation in PBS was confirmed by Nanosight. The same relatively empty samples also had an increase in clarity, except for the 12 mM MgCl₂ samples, which had lower counts compared to 12 mM NaCl and Tris alone.

NTA counts <100 nm had positive correlation and D10 negative correlation to HMMS peak area, however this was not consistent for all samples.

The % HMMS signal at 260 nm (Table 10) was constant per formulation type, regardless of how many full capsids the material contained. While DNA did show maximum absorbance at 260 nm, protein also produces a signal at 260 nm. Based on this data, the % HMMS of AAV3B is likely protein related. The HMMS signal was higher in the relatively full capsid versus the relatively empty capsid samples. Overall HMMS area was higher for the samples in the test formulation. This as attributed to the presence of sucrose in the test formulation which is known to increase the % HMMS in AAV preparations. The % HMMS is not thought to affect potency of an AAV formulation and correlates with a lower likelihood of particulate formation.

DSF: DSF correlation with AAV stability is not yet well understood. There are some reports correlating AAV stability and DSF results (Rieser et al. (2020) J. Pharm. Sci. 109(1):854-862; Rayaprolu et al. (2013) J. Virol. 87(24):13150-13160; Bennett et al. (2017) Molec. Therap. Methods Clin. Dev. 6:171-182; Horowitz et al. (2013) 87(6):2994-3002) though a correlation between potency and DSF results has not been established. However, the following observations could be made (Table 8). Dye-based DNA escape highest peak temperature (using SYBR Gold) corrected well with the intrinsic DSF ratio of 330/350. The highest initial DNA fluorescence was observed in the relatively full Tris sample, which correlated with the elevated turbidity and highest MFI counts. DNA escape onset is expected to be correlated with thermal stability of the AAV material above or close to the reported value. Since no thermal stability was performed in this experiment, this parameter could not be assessed. The best thermal stability is expected in the presence of high levels of NaCl or MgCl₂.

SLS: Static light scattering onset provides information about capsid melt that differs from intrinsic DSF (330 nm/350 nm) or dye-based (SYPRO Orange) DSF. The relatively empty samples with the highest particle counts at >=8 μm (Tris, 12 mM NaCl, 12 mM MgCl₂) had no detectable change in SLS signal during heating from 20° C. to 95° C. (Table 8). SLS signal was not detected for all relatively full samples that had a viral particle titer <40% regardless of the particle aggregation.

### 4. Conclusion

Significant AAV3B viral particle titer loss was observed in the absence of salts (i.e., Tris without salt) after buffer exchange with 50 kDa membrane at 3000 RCF (Table 7). No negative effects were observed when salt concentrations were as high as 2 M (i.e., NaCl or MgCl₂).

CaCl₂ at the lower concentration (i.e., 12 mM) was more stabilizing than equivalent concentrations of NaCl or MgCl₂.

MgCl₂ was more stabilizing than NaCl at the same molarity (i.e., 100 mM), though NaCl is 3 times lower in ionic strength as compared to MgCl₂.

NTA counts <100 nm has a positive correlation and D10 negative correlation to HMMS peak area, however not consistently for all samples.

TABLE 7
Results for T0 studies

| | | DLS | | | | |
|---|---|---|---|---|---|---|
| | | | | | Peak 1 | |
| | | | | | smallest | |
| | | Normalized | Average | | resolved | Peak 1 |
| | | intensity | radius | Polydispersity | radius | Mass |
| Type | Sample name | (cts/sec) | (nm) | (%) | (nm) | (%) |
| AAV3b | Test formulation | 19,937,200 | 13.3 | 20.5 | 0.5^{#} | 100 |
| Fract. 1 | PBS | 69,333,700 | 398.0 | 57.1 | 15.7 | 40 |
| (R = 0.61) | Tris | NSA | NSA | NSA | NSA | NSA |
| Relatively | 12 mM NaCl | NSA | NSA | NSA | NSA | NSA |
| Empty | 12 mM MgCl₂ | 32,714,400 | 165.0 | 57.1 | 12.2 | 57 |
| | 12 mM CaCl₂ | 35,421,500 | 105.8 | 57.1 | 13.9 | 64 |
| | 100 mM NaCl | 22,148,400 | 63.5 | 57.1 | 14.3 | 80 |
| | 100 mM MgCl₂ | 17,697,400 | 11.8 | 3.8 | 12.6 | 100 |
| | 100 mM CaCl₂ | 20,841,700 | 11.9 | 2.7 | 12.3 | 100 |
| | 2000 mM NaCl | 13,655,900 | 12.3 | 4.6 | 12.7 | 100 |
| | 2000 mM MgCl₂ | 10,469,700 | 12.0 | 8.2 | 11.9 | 100 |
| AAV3b | Test formulation | 19,140,900 | 20.1 | 21.6 | 0.4^{#} | 100 |
| Fract. 4 | PBS | 15,954,100 | 587.8 | 57.1 | 18.2 | 75 |
| (R = 0.86) | Tris | 73,666,400 | 194.3 | 57.1 | 282.6 | 25 |
| Relatively | 12 mM NaCl | 19,766,800 | 242.9 | 57.1 | 13.6 | 49 |
| Full | 12 mM MgCl₂ | 24,266,500 | 48.6 | 57.1 | 233.0 | 100 |
| | 12 mM CaCl₂ | 22,308,700 | 12.3 | 57.1 | 12.2 | 76 |
| | 100 mM NaCl | 8,051,770 | 12.1 | 31.8 | 10.7 | 96 |
| | 100 mM MgCl₂ | 17,853,400 | 12.0 | 5.9 | 12.4 | 100 |
| | 100 mM CaCl₂ | 16,612,200 | 13.3 | 19.2 | 10.8 | 84 |
| | 2000 mM NaCl | 11,661,100 | 11.6 | 12.6 | 12.4 | 100 |
| | 2000 mM MgCl₂ | 11,559,000 | 13.3 | 7.3 | 12.3 | 100 |

SEC-HPLC

| | | Monomer | HMMS | Ratio | Viral particle titer |
|---|---|---|---|---|---|
| Type | Sample name | (%) | (%) | (260/280) | VP/mL |
| AAV3b | Test formulation | 97.9 | 2.1 | 0.60 | 1.03E+13 |
| Fract. 1 | PBS | 99.8 | 0.2 | 0.61 | 5.15E+12 |
| (R = 0.61) | Tris | 100.0 | ND | 0.62 | 9.04E+11 |
| Relatively | 12 mM NaCl | 100.0 | ND | 0.62 | 1.14E+12 |
| Empty | 12 mM MgCl₂ | 99.4 | 0.6 | 0.59 | 1.48E+12 |
| | 12 mM CaCl₂ | 99.8 | 0.2 | 0.61 | 4.48E+12 |
| | 100 mM NaCl | 100.0 | ND | 0.60 | 4.05E+12 |
| | 100 mM MgCl₂ | 99.8 | 0.2 | 0.60 | 1.05E+13 |
| | 100 mM CaCl₂ | 99.9 | 0.1 | 0.60 | 1.10E+13 |
| | 2000 mM NaCl | 99.9 | 0.1 | 0.61 | 1.27E+13 |
| | 2000 mM MgCl₂ | 100.0 | ND | 0.61 | 1.11E+13 |
| AAV3b | Test formulation | 98.1 | 1.9 | 0.85 | 9.26E+12 |
| Fract. 4 | PBS | 99.1 | 0.9 | 0.88 | 4.16E+12 |
| (R = 0.86) | Tris | 97.8 | 2.2 | 0.88 | 1.37E+12 |
| Relatively | 12 mM NaCl | 95.2 | 4.8 | 0.88 | 5.13E+11 |
| Full | 12 mM MgCl₂ | 97.6 | 2.4 | 0.87 | 9.82E+11 |
| | 12 mM CaCl₂ | 98.8 | 1.2 | 0.86 | 2.81E+12 |
| | 100 mM NaCl | 99.3 | 0.7 | 0.84 | 2.64E+12 |
| | 100 mM MgCl₂ | 99.5 | 0.5 | 0.85 | 9.00E+12 |
| | 100 mM CaCl₂ | 99.3 | 0.7 | 0.85 | 9.04E+12 |
| | 2000 mM NaCl | 99.7 | 0.3 | 0.88 | 9.62E+12 |
| | 2000 mM MgCl₂ | 99.8 | 0.2 | 0.88 | 1.13E+13 |

DLS was normalized for viscosity; 57.1 is the maximum polydispersity value that the instrument can report; the detection threshold for HMMS is 1%; ND = not detected; NSA = not suitable for analysis.
^{#}Due to the presence of sucrose in the formulation, the intensity of the light scattered from the excipient peak was stronger than the intensity of light scattered from the AAV capsids, which was reported as peak 2 for the test samples (data not shown).

TABLE 8
Results for time 2 weeks at 5° C.

| | | DLS | | | | |
|---|---|---|---|---|---|---|
| | | | | | Peak 1 | |
| | | | | | smallest | |
| | | Normalized | Average | | resolved | Peak 1 |
| | | intensity | radius | Polydispersity | radius | mass |
| Type | Sample name | (cts/sec) | (nm) | (%) | (nm) | (%) |
| AAV3b | Test formulation | 21,115,350 | 14.1 | 49.6 | 0.2^{#} | 100 |
| Fract. 1 | PBS | 28,383,200 | 1278.7 | 57.1 | 137.7 | 70 |
| (R = 0.61) | Tris | 100,655,000 | 593.2 | 57.1 | 309.9 | 16 |
| Relatively | 12 mM NaCl | 46,981,800 | 1703.9 | 57.1 | 466.5 | 100 |
| Empty | 12 mM MgCl₂ | 24,553,100 | 416.7 | 57.1 | 11.1 | 5 |
| | 12 mM CaCl₂ | 48,139,300 | 169.8 | 57.1 | 12.5 | 34 |
| | 100 mM NaCl | 16,512,500 | 22.9 | 57.1 | 11.7 | 87 |
| | 100 mM MgCl₂ | 18,331,100 | 12.0 | 22.6 | 10.9 | 95 |
| | 100 mM CaCl₂ | 19,336,750 | 11.7 | 17.0 | 11.0 | 100 |
| | 2000 mM NaCl | 16,672,550 | 12.6 | 26.8 | 11.5 | 82 |
| | 2000 mM MgCl₂ | 10,880,450 | 12.5 | 12.9 | 12.4 | 100 |
| AAV3b | Test formulation | 12,406,400 | 14.3 | 57.1 | 0.3^{#} | 100 |
| Fract. 4 | PBS | 12,406,400 | 43.1 | 57.1 | 13.0 | 88 |
| (R = 0.86) | Tris | 71,019,700 | 463.0 | 57.1 | 287.7 | 45 |
| Relatively | 12 mM NaCl | 20,244,950 | NSA | NSA | NSA | NSA |
| Full | 12 mM MgCl₂ | 40,416,600 | 250.3 | 57.1 | 12.4 | 4 |
| | 12 mM CaCl₂ | 37,432,450 | 97.8 | 57.1 | 14.3 | 60 |
| | 100 mM NaCl | 9,622,390 | 14.3 | 57.1 | 11.9 | 91 |
| | 100 mM MgCl₂ | 15,798,300 | 12.3 | 11.2 | 11.3 | 100 |
| | 100 mM CaCl₂ | 16,969,700 | 9.8 | 38.6 | 10.5 | 95 |
| | 2000 mM NaCl | 15,871,300 | 15.3 | 39.5 | 13.5 | 67 |
| | 2000 mM MgCl₂ | 12,338,000 | 12.4 | 12.6 | 12.4 | 100 |

SEC-HPLC

| | | Monomer | HMMS | | Viral particle titer |
|---|---|---|---|---|---|
| Type | Sample name | (%) | (%) | Ratio | VP/mL |
| AAV3b | Test formulation | 98.3 | 1.7 | 0.61 | 1.07E+13 |
| Fract. 1 | PBS | 99.5 | 0.5 | 0.61 | 4.78E+12 |
| (R = 0.61) | Tris | 98.9 | 1.1 | 0.62 | 8.02E+11 |
| Relatively | 12 mM NaCl | 98.1 | 1.9 | 0.61 | 1.01E+12 |
| Empty | 12 mM MgCl₂ | 99.0 | 1.0 | 0.61 | 1.43E+12 |
| | 12 mM CaCl₂ | 99.4 | 0.6 | 0.60 | 4.30E+12 |
| | 100 mM NaCl | 100.0 | ND | 0.60 | 4.07E+12 |
| | 100 mM MgCl₂ | 99.7 | 0.3 | 0.60 | 1.10E+13 |
| | 100 mM CaCl₂ | 99.9 | 0.1 | 0.60 | 1.15E+13 |
| | 2000 mM NaCl | 99.9 | 0.1 | 0.61 | 1.28E+13 |
| | 2000 mM MgCl₂ | 100.0 | 0.0 | 0.61 | 1.12E+13 |
| AAV3b | Test formulation | 98.3 | 1.7 | 0.85 | 9.45E+12 |
| Fract. 4 | PBS | 98.5 | 1.5 | 0.87 | 3.83E+12 |
| (R = 0.86) | Tris | 96.3 | 3.7 | 0.87 | 1.01E+12 |
| Relatively | 12 mM NaCl | 96.1 | 3.9 | 0.87 | 4.67E+11 |
| Full | 12 mM MgCl₂ | 96.8 | 3.2 | 0.88 | 9.20E+11 |
| | 12 mM CaCl₂ | 98.1 | 1.9 | 0.85 | 2.83E+12 |
| | 100 mM NaCl | 99.0 | 1.0 | 0.84 | 2.69E+12 |
| | 100 mM MgCl₂ | 99.4 | 0.6 | 0.84 | 9.25E+12 |
| | 100 mM CaCl₂ | 99.0 | 1.0 | 0.85 | 9.42E+12 |
| | 2000 mM NaCl | 99.4 | 0.6 | 0.87 | 9.72E+12 |
| | 2000 mM MgCl₂ | 99.9 | 0.1 | 0.87 | 1.15E+13 |

DSF Capsid

| | | SLS | Ratio 330/350 | Capsid melt with |
|---|---|---|---|---|
| Type | Sample name | onset (° C.) | onset (° C.) | dye (° C.) |
| AAV3b | Test formulation | 75 | 79 | 81 |
| Fract. 1 | PBS | 70 | 71 | 74 |
| (R = 0.61) | Tris | ND | 78 | 82 |
| Relatively | 12 mM NaCl | ND | 77 | 82 |
| Empty | 12 mM MgCl₂ | ND | 78 | 81 |
| | 12 mM CaCl₂ | 82 | 79 | 81 |
| | 100 mM NaCl | 77 | 79 | 81 |
| | 100 mM MgCl₂ | 77 | 78 | 81 |
| | 100 mM CaCl₂ | 77 | 79 | 81 |
| | 2000 mM NaCl | 80 | 81 | 83 |
| | 2000 mM MgCl₂ | 77 | 76 | 79 |
| AAV3b | Test formulation | 77 | 78 | 81 |
| Fract. 4 | PBS | ND | 73 | 80 |
| (R = 0.86) | Tris | ND | 80 | 75 |
| Relatively | 12 mM NaCl | ND | 79 | 85 |
| Full | 12 mM MgCl₂ | ND | 78 | 85 |
| | 12 mM CaCl₂ | ND | 79 | 73 |
| | 100 mM NaCl | ND | 80 | 82 |
| | 100 mM MgCl₂ | 77 | 78 | 83 |
| | 100 mM CaCl₂ | 78 | 79 | 80 |
| | 2000 mM NaCl | 82 | 81 | 81 |
| | 2000 mM MgCl₂ | 77 | 77 | 82 |

DSF DNA (with dye)

| | | | | Initial DNA, |
|---|---|---|---|---|
| | | DNA escape | DNA escape | average |
| | | onset | highest peak | fluorescence |
| Type | Sample name | (° C.) | (° C.) | (RFU) |
| AAV3b | Test formulation | 33 | 78 | 3,046 |
| Fract. 1 | PBS | 37 | 72 | 2,997 |
| (R = 0.61) | Tris | 36 | 78 | 3,031 |
| Relatively | 12 mM NaCl | 36 | 78 | 3,055 |
| Empty | 12 mM MgCl₂ | 36 | 78 | 3,053 |
| | 12 mM CaCl₂ | 35 | 78 | 3,052 |
| | 100 mM NaCl | 37 | 79 | 3,066 |
| | 100 mM MgCl₂ | 35 | 78 | 3,053 |
| | 100 mM CaCl₂ | 35 | 79 | 3,031 |
| | 2000 mM NaCl | 36 | 82 | 2,997 |
| | 2000 mM MgCl₂ | 57 | 78 | 2,998 |
| AAV3b | Test formulation | 36 | 78 | 3,028 |
| Fract. 4 | PBS | 36 | 70 | 3,110 |
| (R = 0.86) | Tris | 38 | 78 | 4,728 |
| Relatively | 12 mM NaCl | 36 | 61 | 3,057 |
| Full | 12 mM MgCl₂ | 33 | 78 | 3,062 |
| | 12 mM CaCl₂ | 35 | 78 | 3,049 |
| | 100 mM NaCl | 35 | 80 | 3,057 |
| | 100 mM MgCl₂ | 36 | 77 | 3,049 |
| | 100 mM CaCl₂ | 36 | 77 | 3,033 |
| | 2000 mM NaCl | 41 | 81 | 2,931 |
| | 2000 mM MgCl₂ | 47 | 76 | 2,960 |

DLS was normalized for viscosity; 57.1 is the maximum polydispersity value that the instrument can report; the detection threshold for HMMS is 1%; ND = not detected; NSA = not suitable for analysis.
^{#}Due to the presence of sucrose in the formulation, the intensity of the light scattered from the excipient peak was stronger than the intensity of light scattered from the AAV capsids, which was reported as peak 2 for the test samples (data not shown).

TABLE 9
Particle-Specific Results for T2 weeks at 5° C.
NTA size (nm)

| Type | Sample name | D10 | D50 | D90 |
|---|---|---|---|---|
| Relatively | Test formulation | 47 | 79 | 154 |
| Empty | PBS | 121 | 190 | 302 |
| | Tris | 85 | 95 | 247 |
| | 12 mM NaCl | 69 | 186 | 304 |
| | 12 mM MgCl₂ | 60 | 138 | 276 |
| | 12 mM CaCl₂ | 76 | 224 | 314 |
| | 100 mM NaCl | 46 | 93 | 260 |
| | 100 mM MgCl₂ | 52 | 86 | 161 |
| | 100 mM CaCl₂ | 59 | 80 | 104 |
| | 2000 mM NaCl | 80 | 141 | 237 |
| | 2000 mM MgCl₂ | 50 | 111 | 204 |
| | Test formulation | 51 | 70 | 138 |
| | PBS | 92 | 129 | 209 |
| | Tris | 60 | 156 | 329 |
| | 12 mM NaCl | 59 | 112 | 240 |
| | 12 mM MgCl₂ | 81 | 108 | 205 |
| | 12 mM CaCl₂ | 56 | 102 | 351 |
| | 100 mM NaCl | 66 | 82 | 126 |
| | 100 mM MgCl₂ | 50 | 78 | 111 |
| | 100 mM CaCl₂ | 50 | 71 | 137 |
| | 2000 mM NaCl | 97 | 136 | 202 |
| | 2000 mM MgCl₂ | 49 | 117 | 176 |

NTA counts/mL

| Type | Sample name | <100 nm | >=100 nm | >=500 nm |
|---|---|---|---|---|
| Relatively | Test formulation | 2.76.E+08 | 138,191,052 | 505,901 |
| Empty | PBS | 4.67.E+07 | 2,217,990,741 | 44,903 |
| | Tris | 1.96.E+08 | 46,031,913 | 13,506 |
| | 12 mM NaCl | 4.41.E+07 | 147,255,889 | 3,771 |
| | 12 mM MgCl₂ | 7.43.E+07 | 114,385,729 | 308,633 |
| | 12 mM CaCl₂ | 3.79.E+07 | 165,430,696 | 128,267 |
| | 100 mM NaCl | 2.49.E+07 | 26,124,513 | ND |
| | 100 mM MgCl₂ | 5.05.E+07 | 29,292,643 | ND |
| | 100 mM CaCl₂ | 8.87.E+07 | 14,286,834 | ND |
| | 2000 mM NaCl | 8.93.E+06 | 40,846,324 | ND |
| | 2000 mM MgCl₂ | 2.61.E+07 | 35,886,052 | 237,666 |
| Relatively | Test formulation | 4.49.E+08 | 100,594,978 | ND |
| Full | PBS | 4.61.E+08 | 2,025,691,571 | 11,631 |
| | Tris | 9.64.E+07 | 208,173,643 | 2,035,788 |
| | 12 mM NaCl | 9.05.E+07 | 128,426,523 | 2,905,878 |
| | 12 mM MgCl₂ | 3.17.E+08 | 505,888,889 | 188,482 |
| | 12 mM CaCl₂ | 1.06.E+08 | 132,524,311 | 1,233 |
| | 100 mM NaCl | 1.04.E+08 | 22,949,200 | 101,133 |
| | 100 mM MgCl₂ | 6.35.E+07 | 11,973,347 | ND |
| | 100 mM CaCl₂ | 2.13.E+08 | 66,633,813 | ND |
| | 2000 mM NaCl | 3.10.E+07 | 229,528,887 | ND |
| | 2000 mM MgCl₂ | 2.71.E+07 | 39,755,686 | ND |

MFI counts/mL

| Type | Sample name | >=1 μm | >=2 μm | >=5 μm | >=8 μm | >=10 μm | >=25 μm |
|---|---|---|---|---|---|---|---|
| Relatively | Test formulation | 8,631 | 2,486 | 687 | 237 | 175 | 0 |
| Empty | PBS | 343,920 | 20,997 | 2,735 | 999 | 575 | 12 |
| | Tris | 215,278 | 29,790 | 7,045 | 2,860 | 1,736 | 62 |
| | 12 mM NaCl | 142,544 | 25,106 | 7,045 | 3,298 | 2,036 | 237 |
| | 12 mM MgCl₂ | 50,675 | 14,976 | 4,959 | 2,486 | 1,736 | 112 |
| | 12 mM CaCl₂ | 27,317 | 7,245 | 2,011 | 837 | 575 | 50 |
| | 100 mM NaCl | 186,936 | 19,848 | 1,262 | 512 | 300 | 37 |
| | 100 mM MgCl₂ | 7,594 | 2,636 | 962 | 412 | 237 | 25 |
| | 100 mM CaCl₂ | 8,631 | 2,773 | 762 | 312 | 200 | 12 |
| | 2000 mM NaCl | 7,469 | 2,436 | 699 | 275 | 200 | 12 |
| | 2000 mM MgCl₂ | 1,774 | 612 | 112 | 25 | 25 | 0 |
| Relatively | Test formulation | 6,708 | 2,073 | 587 | 175 | 125 | 12 |
| Full | PBS | 109,182 | 10,854 | 1,536 | 712 | 550 | 37 |
| | Tris | 469,090 | 54,684 | 11,954 | 5,771 | 3,835 | 300 |
| | 12 mM NaCl | 217,251 | 39,721 | 10,105 | 4,647 | 2,898 | 362 |
| | 12 mM MgCl₂ | 58,944 | 13,403 | 3,560 | 1,474 | 837 | 50 |
| | 12 mM CaCl₂ | 17,362 | 5,296 | 1,574 | 637 | 412 | 25 |
| | 100 mM NaCl | 48,726 | 8,394 | 974 | 312 | 150 | 12 |
| | 100 mM MgCl₂ | 7,557 | 2,236 | 662 | 300 | 200 | 12 |
| | 100 mM CaCl₂ | 12,853 | 5,159 | 1,974 | 1,174 | 762 | 50 |
| | 2000 mM NaCl | 3,785 | 999 | 212 | 125 | 50 | 0 |
| | 2000 mM MgCl₂ | 2,586 | 600 | 87 | 0 | 0 | 0 |

Appearance

| Type | Sample name | Color | Clarity | Particle description |
|---|---|---|---|---|
| Relatively | Test formulation | <=B9 | I | 3 very small white |
| Empty | PBS | <=B9 | II | 4 white, one large elongated |
| | Tris | <=B9 | II | 3 medium |
| | 12 mM NaCl | <=B9 | II | 2 fibers, likely environmental |
| | 12 mM MgCl₂ | <=B9 | I | EFVP |
| | 12 mM CaCl₂ | <=B9 | I | 5, one is large plastic-like |
| | 100 mM NaCl | <=B9 | I | 2 fibers, likely environmental |
| | 100 mM MgCl₂ | <=B9 | I | ~7 white large to medium |
| | 100 mM CaCl₂ | <=B9 | I | 2 white medium |
| | 2000 mM NaCl | <=B9 | I | 2 tiny white |
| | 2000 mM MgCl₂ | <=B9 | I | 3 medium to large white |
| Relatively | Test formulation | <=B9 | I | 5 very small white |
| Full | PBS | <=B9 | I | 3 white large |
| | Tris | <=B9 | II | 10 of various length, white fluffy |
| | 12 mM NaCl | <=B9 | I | 3 small white |
| | 12 mM MgCl₂ | <=B9 | I | 5 white, one is very large, |
| | | | | resembles PE |
| | 12 mM CaCl₂ | <=B9 | I | 2 fibers, likely environmental |
| | 100 mM NaCl | <=B9 | I | 1 large fiber, likely environmental |
| | 100 mM MgCl₂ | <=B9 | I | 2 white large |
| | 100 mM CaCl₂ | <=B9 | I | 3 tiny white |
| | 2000 mM NaCl | <=B9 | I | 1 large transparent, likely |
| | | | | environmental |
| | 2000 mM MgCl₂ | <=B9 | I | EFVP |

ND = not detected; DLS was normalized for viscosity; detection threshold for HMMS is 1.0%. D10, D50 and D90 represent particle size values at the 10^{th}, 50^{th} and 90^{th} percentile as defined in Hubert et al. (2020) J. Pharma. Sci. 109: 830-844.

TABLE 10
SEC % HMMS and peak area

| SEC % HMMS | | | | |
|---|---|---|---|---|
| | 214 nm | Area 214 nm | Area 260 nm | Area 280 nm |

| Type | Sample name | % | % | T0 | T2 wk | T0 | T2 wk | T0 | T2 wk |
|---|---|---|---|---|---|---|---|---|---|
| Relatively | Test formulation | 2.1 | 1.7 | 65,736 | 54,409 | 4345 | 3,989 | 5876 | 4643 |
| Empty | PBS | 0.2 | 0.5 | 3,808 | 7,490 | ND | 371 | ND | 444 |
| | Tris | ND | 1.1 | ND | 3,118 | ND | ND | ND | ND |
| | 12 mM NaCl | ND | 1.9 | ND | 6,735 | ND | ND | ND | ND |
| | 12 mM MgCl₂ | 0.6 | 1.0 | 2,799 | 4,722 | ND | ND | ND | ND |
| | 12 mM CaCl₂ | 0.2 | 0.6 | 2,292 | 7,693 | ND | ND | ND | ND |
| | 100 mM NaCl | ND | ND | ND | ND | ND | ND | ND | ND |
| | 100 mM MgCl₂ | 0.2 | 0.3 | 6,998 | 8,249 | 477 | 502 | 408 | 543 |
| | 100 mM CaCl₂ | 0.1 | 0.1 | 2,407 | 4,700 | ND | ND | ND | ND |
| | 2000 mM NaCl | 0.1 | 0.1 | 4,363 | 2,806 | ND | ND | ND | ND |
| | 2000 mM MgCl₂ | 0.0 | 0.0 | 739 | 889 | ND | ND | ND | ND |
| Relatively | Test formulation | 1.9 | 1.7 | 53,000 | 46,952 | 4830 | 3,676 | ND | 1,094 |
| Full | PBS | 0.9 | 1.5 | 11,432 | 17,529 | ND | 835 | ND | 1,169 |
| | Tris | 2.2 | 3.7 | 10,244 | 13,594 | ND | 695 | 1456 | 1,038 |
| | 12 mM NaCl | 4.8 | 3.9 | 10,495 | 8,018 | 661 | 1,029 | 890 | 852 |
| | 12 mM MgCl₂ | 2.4 | 3.2 | 8,497 | 10,806 | 542 | 917 | 542 | 1,651 |
| | 12 mM CaCl₂ | 1.2 | 1.9 | 10,976 | 17,125 | 452 | 1,284 | 367 | 870 |
| | 100 mM NaCl | 0.7 | 1.0 | 5,942 | 8,455 | 259 | 595 | 682 | 1,318 |
| | 100 mM MgCl₂ | 0.5 | 0.6 | 12,924 | 15,873 | 473 | 978 | 914 | 2,114 |
| | 100 mM CaCl₂ | 0.7 | 1.0 | 18,403 | 27,086 | 647 | 1,998 | ND | 1,001 |
| | 2000 mM NaCl | 0.3 | 0.6 | 9,390 | 17,921 | ND | 667 | ND | ND |
| | 2000 mM MgCl₂ | 0.2 | 0.1 | 5,384 | 4,902 | ND | 3,676 | ND | 1,094 |

AAV area

| | | Area 214 nm | | Area 260 nm | | Area 280 nm | |
|---|---|---|---|---|---|---|---|
| Type | Sample name | T0 | T2 wk | T0 | T2 wk | T0 | T2 wk |
| Relatively | Test formulation | 3,108,297 | 3,165,844 | 141003 | 144,483 | 234,577 | 239,001 |
| Empty | PBS | 1,578,988 | 1,443,225 | 72646 | 66,618 | 118,338 | 109,571 |
| | Tris | 324,180 | 292,012 | 15539 | 13,670 | 25,165 | 22,225 |
| | 12 mM NaCl | 393,689 | 353,539 | 18831 | 16,146 | 30,167 | 26,353 |
| | 12 mM MgCl₂ | 494,542 | 474,689 | 22379 | 21,842 | 37,710 | 35,770 |
| | 12 mM CaCl₂ | 1,380,974 | 1,304,245 | 62941 | 59,268 | 104,003 | 98,455 |
| | 100 mM NaCl | 1,253,455 | 1,238,635 | 56657 | 56,067 | 94,057 | 93,381 |
| | 100 mM MgCl₂ | 3,147,668 | 3,254,319 | 142960 | 146,813 | 236,932 | 244,737 |
| | 100 mM CaCl₂ | 3,313,147 | 3,379,923 | 149998 | 152,618 | 249,336 | 254,041 |
| | 2000 mM NaCl | 3,816,385 | 3,775,556 | 176092 | 175,122 | 288,596 | 286,136 |
| | 2000 mM MgCl₂ | 3,321,630 | 3,307,112 | 154359 | 152,283 | 251,146 | 250,678 |
| Relatively | Test formulation | 2,792,575 | 2,794,747 | 224568 | 223,543 | 263,220 | 263,426 |
| Full | PBS | 1,287,049 | 1,169,739 | 107425 | 97,292 | 122,555 | 112,289 |
| | Tris | 462,306 | 351,225 | 39571 | 29,676 | 44,953 | 34,311 |
| | 12 mM NaCl | 208,849 | 195,305 | 18859 | 17,762 | 21,339 | 20,313 |
| | 12 mM MgCl₂ | 347,334 | 326,163 | 28665 | 27,547 | 32,901 | 31,412 |
| | 12 mM CaCl₂ | 885,738 | 880,371 | 71603 | 70,770 | 83,657 | 83,375 |
| | 100 mM NaCl | 837,325 | 837,594 | 65992 | 66,923 | 78,615 | 79,322 |
| | 100 mM MgCl₂ | 2,713,241 | 2,736,112 | 215728 | 216,699 | 253,743 | 257,849 |
| | 100 mM CaCl₂ | 2,727,177 | 2,786,467 | 217665 | 223,340 | 255,964 | 264,019 |
| | 2000 mM NaCl | 2,898,429 | 2,872,641 | 244219 | 238,742 | 278,601 | 274,372 |
| | 2000 mM MgCl₂ | 3,388,951 | 3,379,794 | 287908 | 283,640 | 328,796 | 326,024 |

ND = not detected; detection threshold for HMMS is 1.0%.

### EQUIVALENTS

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, textbooks, and the like, and the references cited therein, to the extent that they are not already, are hereby incorporated herein by reference in their entirety.

SEQUENCES

| NO. | DESCRIPTION | SEQUENCE |
|---|---|---|
| 1 | amino acid | MPEQERQITAREGASRKILSKLSLPTRAWEPAMKKSFAFDNVGYEGGLDGLGPSSQPQ |
| | sequence of | KCFLQIKGMTCASCVSNIERNLQKEAGVLSVLVALMAGKAEIKYDPEVIQPLEIAQFI |
| | copper | QDLGFEAAVMEDYAGSDGNIELTITGMTCASCVHNIESKLTRINGITYASVALATSKA |
| | transporting | LVKFDPEIIGPRDIIKIIEEIGFHASLAQRNPNAHHLDHKMEIKOWKKSFLCSLVFGI |
| | ATPase 2 | PVMALMIYMLIPSNEPHQSMVLDHNIIPGLSILNLIFFILCTFVOLLGGWYFYVQAYK |
| | with | SLRHRSANMDVLIVLATSIAYVYSLVILVVAVAEKAERSPVTFFDTPPMLFVFIALGR |
| | deletion of | WLEHLAKSKTSEALAKLMSLQATEATVVTLGEDNLIIREEQVPMELVQRGDIVKVVPG |
| | MBS 1-4 | GKFPVDGKVLEGNTMADESLITGEAMPVTKKPGSTVIAGSINAHGSVLIKATHVGNDT |
| | | TLAQIVKLVEEAQMSKAPIQQLADRFSGYFVPFIIIMSTLTLVVWIVIGFIDFGVVQR |
| | | YFPNPNKHISQTEVIIRFAFQTSITVLCIACPCSLGLATPTAVMVGTGVAAQNGILIK |
| | | GGKPLEMAHKIKTVMFDKTGTITHGVPRVMRVLLLGDVATLPLRKVLAVVGTAEASSE |
| | | HPLGVAVTKYCKEELGTETLGYCTDFQAVPGCGIGCKVSNVEGILAHSERPLSAPASH |
| | | LNEAGSLPAEKDAVPQTFSVLIGNREWLRRNGLTISSDVSDAMTDHEMKGQTAILVAI |
| | | DGVLCGMIAIADAVKQEAALAVHTLQSMGVDVVLITGDNRKTARAIATQVGINKVFAE |
| | | VLPSHKVAKVQELQNKGKKVAMVGDGVNDSPALAQADMGVAIGTGTDVAIEAADVVLI |
| | | RNDLLDVVASIHLSKRTVRRIRINLVLALIYNLVGIPIAAGVFMPIGIVLQPWMGSAA |
| | | MAASSVSVVLSSLQLKCYKKPDLERYEAQAHGHMKPLTASQVSVHIGMDDRWRDSPRA |
| | | TPWDQVSYVSQVSLSSLTSDKPSRHSAAADDDGDKWSLLLNGRDEEQYI |
| 2 | nucleotide | ATGCCTGAGCAGGAGAGACAGATCACAGCCAGAGAAGGGGCCAGTCGGAAAATCTTAT |
| | sequence of | CTAAGCTTTCTTTGCCTACCCGTGCCTGGGAACCAGCAATGAAGAAGAGTTTTGCTTT |
| | ATP 7B with | TGACAATGTTGGCTATGAAGGTGGTCTGGATGGCCTGGGCCCTTCTTCTCAGCCGCAG |
| | deletion of | AAGTGCTTCTTACAGATCAAAGGCATGACCTGTGCATCCTGTGTGTCTAACATAGAAA |
| | MBS 1-4 | GGAATCTGCAGAAAGAAGCTGGTGTTCTCTCCGTGTTGGTTGCCTTGATGGCAGGAAA |
| | | GGCAGAGATCAAGTATGACCCAGAGGTCATCCAGCCCCTCGAGATAGCTCAGTTCATC |
| | | CAGGACCTGGGTTTTGAGGCAGCAGTCATGGAGGACTACGCAGGCTCCGATGGCAACA |
| | | TTGAGCTGACAATCACAGGGATGACCTGCGCGTCCTGTGTCCACAACATAGAGTCCAA |
| | | ACTCACGAGGACAAATGGCATCACTTATGCCTCCGTTGCCCTTGCCACCAGCAAAGCC |
| | | CTTGTTAAGTTTGACCCGGAAATTATCGGTCCACGGGATATTATCAAAATTATTGAGG |
| | | AAATTGGCTTTCATGCTTCCCTGGCCCAGAGAAACCCCAACGCTCATCACTTGGACCA |
| | | CAAGATGGAAATAAAGCAGTGGAAGAAGTCTTTCCTGTGCAGCCTGGTGTTTGGCATC |
| | | CCTGTCATGGCCTTAATGATCTATATGCTGATACCCAGCAACGAGCCCCACCAGTCCA |
| | | TGGTCCTGGACCACAACATCATTCCAGGACTGTCCATTCTAAATCTCATCTTCTTTAT |
| | | CTTGTGTACCTTTGTCCAGCTCCTCGGTGGGTGGTACTTCTACGTTCAGGCCTACAAA |
| | | TCTCTGAGACACAGGTCAGCCAACATGGACGTGCTCATCGTCCTGGCCACAAGCATTG |
| | | CTTATGTTTATTCTCTGGTCATCCTGGTGGTTGCTGTGGCTGAGAAGGCGGAGAGGAG |
| | | CCCTGTGACATTCTTCGACACGCCCCCCATGCTCTTTGTGTTCATTGCCCTGGGCCGG |
| | | TGGCTGGAACACTTGGCAAAGAGCAAAACCTCAGAAGCCCTGGCTAAACTCATGTCTC |
| | | TCCAAGCCACAGAAGCCACCGTTGTGACCCTTGGTGAGGACAATTTAATCATCAGGGA |
| | | GGAGCAAGTCCCCATGGAGCTGGTGCAGCGGGGCGATATCGTCAAGGTGGTCCCTGGG |
| | | GGAAAGTTTCCAGTGGATGGGAAAGTCCTGGAAGGCAATACCATGGCTGATGAGTCCC |
| | | TCATCACAGGAGAAGCCATGCCAGTCACTAAGAAACCCGGAAGCACTGTAATTGCGGG |
| | | GTCTATAAATGCACATGGCTCTGTGCTCATTAAAGCTACCCACGTGGGCAATGACACC |
| | | ACTTTGGCTCAGATTGTGAAACTGGTGGAAGAGGCTCAGATGTCAAAGGCACCCATTC |
| | | AGCAGCTGGCTGACCGGTTTAGTGGATATTTTGTCCCATTTATCATCATCATGTCAAC |
| | | TTTGACGTTGGTGGTATGGATTGTAATCGGTTTTATCGATTTTGGTGTTGTTCAGAGA |
| | | TACTTTCCTAACCCCAACAAGCACATCTCCCAGACAGAGGTGATCATCCGGTTTGCTT |
| | | TCCAGACGTCCATCACGGTGCTGTGCATTGCCTGCCCCTGCTCCCTGGGGCTGGCCAC |
| | | GCCCACGGCTGTCATGGTGGGCACCGGGGTGGCCGCGCAGAACGGCATCCTCATCAAG |
| | | GGAGGCAAGCCCCTGGAGATGGCGCACAAGATAAAGACTGTGATGTTTGACAAGACTG |
| | | GCACCATTACCCATGGCGTCCCCAGGGTCATGCGGGTGCTCCTGCTGGGGGATGTGGC |
| | | CACACTGCCCCTCAGGAAGGTTCTGGCTGTGGTGGGGACTGCGGAGGCCAGCAGTGAA |
| | | CACCCCTTGGGCGTGGCAGTCACCAAATACTGTAAAGAGGAACTTGGAACAGAGACCT |
| | | TGGGATACTGCACGGACTTCCAGGCAGTGCCAGGCTGTGGAATTGGGTGCAAAGTCAG |
| | | CAACGTGGAAGGCATCCTGGCCCACAGTGAGCGCCCTTTGAGTGCACCGGCCAGTCAC |
| | | CTGAATGAGGCTGGCAGCCTTCCCGCAGAAAAAGATGCAGTCCCCCAGACCTTCTCTG |
| | | TGCTGATTGGAAACCGTGAGTGGCTGAGGCGCAACGGTTTAACCATTTCTAGCGATGT |
| | | CAGTGACGCTATGACAGACCACGAGATGAAAGGACAGACAGCCATCCTGGTGGCTATT |
| | | GACGGTGTGCTCTGTGGGATGATCGCAATCGCAGACGCTGTCAAGCAGGAGGCTGCCC |
| | | TGGCTGTGCACACGCTGCAGAGCATGGGTGTGGACGTGGTTCTGATCACGGGGGACAA |
| | | CCGGAAGACAGCCAGAGCTATTGCCACCCAGGTTGGCATCAACAAAGTCTTTGCAGAG |
| | | GTGCTGCCTTCGCACAAGGTGGCCAAGGTCCAGGAGCTCCAGAATAAAGGGAAGAAAG |
| | | TCGCCATGGTGGGGGATGGGGTCAATGACTCCCCGGCCTTGGCCCAGGCAGACATGGG |
| | | TGTGGCCATTGGCACCGGCACGGATGTGGCCATCGAGGCAGCCGACGTCGTCCTTATC |
| | | AGAAATGATTTGCTGGATGTGGTGGCTAGCATTCACCTTTCCAAGAGGACTGTCCGAA |
| | | GGATACGCATCAACCTGGTCCTGGCACTGATTTATAACCTGGTTGGGATACCCATTGC |
| | | AGCAGGTGTCTTCATGCCCATCGGCATTGTGCTGCAGCCCTGGATGGGCTCAGCGGCC |
| | | ATGGCAGCCTCCTCTGTGTCTGTGGTGCTCTCATCCCTGCAGCTCAAGTGCTATAAGA |
| | | AGCCTGACCTGGAGAGGTATGAGGCACAGGCGCATGGCCACATGAAGCCCCTGACGGC |
| | | ATCCCAGGTCAGTGTGCACATAGGCATGGATGACAGGTGGCGGGACTCCCCCAGGGCC |
| | | ACACCATGGGACCAGGTCAGCTATGTCAGCCAGGTGTCGCTGTCCTCCCTGACGTCCG |
| | | ACAAGCCATCTCGGCACAGCGCTGCAGCAGACGATGATGGGGACAAGTGGTCTCTGCT |
| | | CCTGAATGGCAGGGATGAGGAGCAGTACATCTGA |
| 3 | promoter | cgccaccccctccaccttggacacaggacgctgtggtttctgagccaggtacaatgac |
| | | tcctttcggtaagtgcagtggaagctgtacactgcccaggcaaagcgtccgggcagcg |
| | | taggcgggcgactcagatcccagccagtggacttagcccctgtttgctcctccgataa |
| | | ctggggtgaccttggttaatattcaccagcagcctcccccgttgcccctctggatcca |
| | | ctgcttaaatacggacgaggacagggccctgtctcctcagcttcaggcaccaccactg |
| | | acctgggacagtgaa |
| 4 | polyA | aataaagacctcttattttcattcatcaggtgtggttggtttttttgtgtgggggc |
| 5 | ITR | cctgcaggcagctgcgcgctcgctcgctcactgaggccgcccgggcaaagcccgggcg |
| | | tcgggcgacctttggtcgcccggcctcagtgagcgagcgagcgcgcagagagggagtg |
| | | gccaactccatcactaggggttcct |
| 6 | ITR | ctgcgcgctcgctcgctcactgaggccgcccgggcaaagcccgggcgtcgggcgacct |
| | | ttggtcgcccggcctcagtgagcgagcgagcgcgcagagagggagtggccaactccat |
| | | cactaggggttcct |
| 7 | ITR | aggaacccctagtgatggagttggccactccctctctgcgcgctcgctcgctcactga |
| | | ggccgggcgaccaaaggtcgcccgacgcccgggctttgcccgggcggcctcagtgagc |
| | | gagcgagcgcgcagctgcctgcag |
| 8 | ITR | aggaacccctagtgatggagttggccactccctctctgcgcgctcgctcgctcactga |
| | | ggccgggcgaccaaaggtcgcccgacgcccgggctttgcccgggcggcctcagtgagc |
| | | gagcgagcgcgcag |
| 9 | ATP7B | cctgcaggca gctgcgcgct cgctcgctca ctgaggccgc ccgggcaaag cccgggcgtc |
| | transgene | gggcgacctt tggtcgcccg gcctcagtga gcgagcgagc gcgcagagag ggagtggcca |
| | cassette | actccatcac taggggttcc tgcggccgca cgcgtcgcca ccccctccac cttggacaca |
| | | ggacgctgtg gtttctgagc caggtacaat gactcctttc ggtaagtgca gtggaagctg |
| | | tacactgccc aggcaaagcg tccgggcage gtaggcgggc gactcagatc ccagccagtg |
| | | gacttagccc ctgtttgctc ctccgataac tggggtgacc ttggttaata ttcaccagca |
| | | gcctcccccg ttgcccctct ggatccactg cttaaatacg gacgaggaca gggccctgtc |
| | | tcctcagctt caggcaccac cactgacctg ggacagtgaa gcggccgcca ccatgcctga |
| | | gcaggagaga cagatcacag ccagagaagg ggccagtcgg aaaatcttat ctaagctttc |
| | | tttgcctacc cgtgcctggg aaccagcaat gaagaagagt tttgcttttg acaatgttgg |
| | | ctatgaaggt ggtctggatg gcctgggccc ttcttctcag ccgcagaagt gcttcttaca |
| | | gatcaaaggc atgacctgtg catcctgtgt gtctaacata gaaaggaatc tgcagaaaga |
| | | agctggtgtt ctctccgtgt tggttgcctt gatggcagga aaggcagaga tcaagtatga |
| | | cccagaggtc atccagcccc tcgagatagc tcagttcate caggacctgg gttttgaggc |
| | | agcagtcatg gaggactacg caggctccga tggcaacatt gagctgacaa tcacagggat |
| | | gacctgcgcg tcctgtgtcc acaacataga gtccaaactc acgaggacaa atggcatcac |
| | | ttatgcctcc gttgcccttg ccaccagcaa agcccttgtt aagtttgacc cggaaattat |
| | | cggtccacgg gatattatca aaattattga ggaaattggc tttcatgctt ccctggccca |
| | | gagaaacccc aacgctcatc acttggacca caagatggaa ataaagcagt ggaagaagtc |
| | | tttcctgtgc agcctggtgt ttggcatccc tgtcatggcc ttaatgatct atatgctgat |
| | | acccagcaac gagccccacc agtccatggt cctggaccac aacatcattc caggactgtc |
| | | cattctaaat ctcatcttct ttatcttgtg tacctttgtc cagctcctcg gtgggTggta |
| | | cttctacgtt caggcctaca aatctctgag acacaggtca gccaacatgg acgtgctcat |
| | | cgtcctggcc acaagcattg cttatgttta ttctctggtc atcctggtgg ttgctgtggc |
| | | tgagaaggcg gagaggagcc ctgtgacatt cttcgacacg ccccccatgc tctttgtgtt |
| | | cattgccctg ggccggtggc tggaacactt ggcaaagage aaaacctcag aagccctggc |
| | | taaactcatg tctctccaag ccacagaagc caccgttgtg acccttggtg aggacaattt |
| | | aatcatcagg gaggagcaag tccccatgga gctggtgcag cggggcgata tcgtcaaggt |
| | | ggtccctggg ggaaagtttc cagtggatgg gaaagtcctg gaaggcaata ccatggctga |
| | | tgagtccctc atcacaggag aagccatgcc agtcactaag aaacccggaa gcactgtaat |
| | | tgcggggtct ataaatgcac atggctctgt gctcattaaa gctacccacg tgggcaatga |
| | | caccactttg gctcagattg tgaaactggt ggaagaggct cagatgtcaa aggcacccat |
| | | tcagcagctg gctgaccggt ttagtggata ttttgtccca tttatcatca tcatgtcaac |
| | | tttgacgttg gtggtatgga ttgtaatcgg ttttatcgat tttggtgttg ttcagagata |
| | | ctttcctaac cccaacaagc acatctccca gacagaggtg atcatccggt ttgctttcca |
| | | gacgtccatc acggtgCtgt gcattgcctg cccctgctcc ctggggctgg ccacgcccac |
| | | ggctgtcatg gtgggcaccg gggtggccgc gcagaacggc atcctcatca agggaggcaa |
| | | gcccctggag atggcgcaca agataaagac tgtgatgttt gacaagactg gcaccattac |
| | | ccatggcgtc cccagggtca tgcgggtgct cctgctgggg gatgtggcca cactgcccct |
| | | caggaaggtt ctggctgtgg tggggactgc ggaggccagc agtgaacacc ccttgggcgt |
| | | ggcagtcacc aaatactgta aagaggaact tggaacagag accttgggat actgcacgga |
| | | cttccaggca gtgccaggct gtggaattgg gtgcaaagtc agcaacgtgg aaggcatcct |
| | | ggcccacagt gagcgccctt tgagtgcacc ggccagtcac ctgaatgagg ctggcagcct |
| | | tcccgcagaa aaagatgcag tcccccagac cttctctgtg ctgattggaa accgtgagtg |
| | | gctgaggcgc aacggtttaa ccatttctag cgatgtcagt gacgctatga cagaccacga |
| | | gatgaaagga cagacagcca tcctggtggc tattgacggt gtgctctgtg ggatgatcgc |
| | | aatcgcagac gctgtcaagc aggaggctgc cctggctgtg cacacgctgc agagcatggg |
| | | tgtggacgtg gttctgatca cgggggacaa ccggaagaca gccagagcta ttgccaccca |
| | | ggttggcatc aacaaagtct ttgcagaggt gctgccttcg cacaaggtgg ccaaggtcca |
| | | ggagctccag aataaaggga agaaagtcgc catggtgggg gatggggtca atgactcccc |
| | | ggccttggcc caggcagaca tgggtgtggc cattggcacc ggcacggatg tggccatcga |
| | | ggcagccgac gtcgtcctta tcagaaatga tttgctggat gtggtggcta gcattcacct |
| | | ttccaagagg actgtccgaa ggatacgcat caacctggtc ctggcactga tttataacct |
| | | ggttgggata cccattgcag caggtgtctt catgcccatc ggcattgtgc tgcagccctg |
| | | gatgggctca gcggccatgg cagcctcctc tgtgtctgtg gtgctctcat ccctgcagct |
| | | caagtgctat aagaagcctg acctggagag gtatgaggca caggcgcatg gccacatgaa |
| | | gcccctgacg gcatcccagg tcagtgtgca cataggcatg gatgacaggt ggcgggactc |
| | | ccccagggcc acaccatggg accaggtcag ctatgtcage caggtgtcgc tgtcctccct |
| | | gacgtccgac aagccatctc ggcacagcgc tgcagcagac gatgatgggg acaagtggtc |
| | | tctgctcctg aatggcaggg atgaggagca gtacatctga ggtaccaata aagacctctt |
| | | attttcattc atcaggtgtg gttggttttt ttgtgtgggg gcggatccat cggatcccgt |
| | | gcggaccgag cggccgcagg aacccctagt gatggagttg gccactccct ctctgcgcgc |
| | | tcgctcgctc actgaggccg ggcgaccaaa ggtcgcccga cgcccgggct ttgcccgggc |
| | | ggcctcagtg agcgagcgag cgcgcagctg cctgcag |
| 10 | AAV3B VP1 | MAADGYLPDWLEDNLSEGIREWWALKPGVPQPKANQQHQDNRRGLVLPGYKYLGPGNG |
| | capsid | LDKGEPVNEADAAALEHDKAYDQQLKAGDNPYLKYNHADAEFQERLQEDTSFGGNLGR |
| | polypeptide | AVFQAKKRILEPLGLVEEAAKTAPGKKRPVDQSPQEPDSSSGVGKSGKQPARKRLNFG |
| | (AAB95452.1) | QTGDSESVPDPQPLGEPPAAPTSLGSNTMASGGGAPMADNNEGADGVGNSSGNWHCDS |
| | | QWLGDRVITTSTRTWALPTYNNHLYKQISSQSGASNDNHYFGYSTPWGYFDFNRFHCH |
| | | FSPRDWQRLINNNWGFRPKKLSFKLFNIQVKEVTQNDGITTIANNLTSTVQVFTDSEY |
| | | QLPYVLGSAHQGCLPPFPADVFMVPQYGYLTLNNGSQAVGRSSFYCLEYFPSQMLRTG |
| | | NNFQFSYTFEDVPFHSSYAHSQSLDRLMNPLIDQYLYYLNRTQGTTSGTTNQSRLLES |
| | | QAGPQSMSLQARNWLPGPCYRQQRLSKTANDNNNSNFPWTAASKYHLNGRDSLVNPGP |
| | | AMASHKDDEEKFFPMHGNLIFGKEGTTASNAELDNVMITDEEEIRTTNPVATEQYGTV |
| | | ANNLQSSNTAPTTRTVNDQGALPGMVWQDRDVYLQGPIWAKIPHTDGHFHPSPLMGGF |
| | | GLKHPPPQIMIKNTPVPANPPTTFSPAKFASFITQYSTGQVSVEIEWELQKENSKRWN |
| | | PEIQYTSNYNKSVNVDFTVDINGVYSEPRPIGTRYLTRNL |
| 11 | AAV3B VP1 | ATGGCTGCTGACGGTTATCTTCCAGATTGGCTCGAGGACAACCTTTCTGAAGGCATTC |
| | capsid nt | GTGAGTGGTGGGCTCTGAAACCTGGAGTCCCTCAACCCAAAGCGAACCAACAACACCA |
| | sequence | GGACAACCGTCGGGGTCTTGTGCTTCCGGGTTACAAATACCTCGGACCCGGTAACGGA |
| | (nt 2208- | CTCGACAAAGGAGAGCCGGTCAACGAGGCGGACGCGGCAGCCCTCGAACACGACAAAG |
| | 4418 of | CTTACGACCAGCAGCTCAAGGCCGGTGACAACCCGTACCTCAAGTACAACCACGCCGA |
| | AF028705.1) | CGCCGAGTTTCAGGAGCGTCTTCAAGAAGATACGTCTTTTGGGGGCAACCTTGGCAGA |
| | | GCAGTCTTCCAGGCCAAAAAGAGGATCCTTGAGCCTCTTGGTCTGGTTGAGGAAGCAG |
| | | CTAAAACGGCTCCTGGAAAGAAGAGGCCTGTAGATCAGTCTCCTCAGGAACCGGACTC |
| | | ATCATCTGGTGTTGGCAAATCGGGCAAACAGCCTGCCAGAAAAAGACTAAATTTCGGT |
| | | CAGACTGGCGACTCAGAGTCAGTCCCAGACCCTCAACCTCTCGGAGAACCACCAGCAG |
| | | CCCCCACAAGTTTGGGATCTAATACAATGGCTTCAGGCGGTGGCGCACCAATGGCAGA |
| | | CAATAACGAGGGTGCCGATGGAGTGGGTAATTCCTCAGGAAATTGGCATTGCGATTCC |
| | | CAATGGCTGGGCGACAGAGTCATCACCACCAGCACCAGAACCTGGGCCCTGCCCACTT |
| | | ACAACAACCATCTCTACAAGCAAATCTCCAGCCAATCAGGAGCTTCAAACGACAACCA |
| | | CTACTTTGGCTACAGCACCCCTTGGGGGTATTTTGACTTTAACAGATTCCACTGCCAC |
| | | TTCTCACCACGTGACTGGCAGCGACTCATTAACAACAACTGGGGATTCCGGCCCAAGA |
| | | AACTCAGCTTCAAGCTCTTCAACATCCAAGTTAAAGAGGTCACGCAGAACGATGGCAC |
| | | GACGACTATTGCCAATAACCTTACCAGCACGGTTCAAGTGTTTACGGACTCGGAGTAT |
| | | CAGCTCCCGTACGTGCTCGGGTCGGCGCACCAAGGCTGTCTCCCGCCGTTTCCAGCGG |
| | | ACGTCTTCATGGTCCCTCAGTATGGATACCTCACCCTGAACAACGGAAGTCAAGCGGT |
| | | GGGACGCTCATCCTTTTACTGCCTGGAGTACTTCCCTTCGCAGATGCTAAGGACTGGA |
| | | AATAACTTCCAATTCAGCTATACCTTCGAGGATGTACCTTTTCACAGCAGCTACGCTC |
| | | ACAGCCAGAGTTTGGATCGCTTGATGAATCCTCTTATTGATCAGTATCTGTACTACCT |
| | | GAACAGAACGCAAGGAACAACCTCTGGAACAACCAACCAATCACGGCTGCTTTTTAGC |
| | | CAGGCTGGGCCTCAGTCTATGTCTTTGCAGGCCAGAAATTGGCTACCTGGGCCCTGCT |
| | | ACCGGCAACAGAGACTTTCAAAGACTGCTAACGACAACAACAACAGTAACTTTCCTTG |
| | | GACAGCGGCCAGCAAATATCATCTCAATGGCCGCGACTCGCTGGTGAATCCAGGACCA |
| | | GCTATGGCCAGTCACAAGGACGATGAAGAAAAATTTTTCCCTATGCACGGCAATCTAA |
| | | TATTTGGCAAAGAAGGGACAACGGCAAGTAACGCAGAATTAGATAATGTAATGATTAC |
| | | GGATGAAGAAGAGATTCGTACCACCAATCCTGTGGCAACAGAGCAGTATGGAACTGTG |
| | | GCAAATAACTTGCAGAGCTCAAATACAGCTCCCACGACTAGAACTGTCAATGATCAGG |
| | | GGGCCTTACCTGGCATGGTGTGGCAAGATCGTGACGTGTACCTTCAAGGACCTATCTG |
| | | GGCAAAGATTCCTCACACGGATGGACACTTTCATCCTTCTCCTCTGATGGGAGGCTTT |
| | | GGACTGAAACATCCGCCTCCTCAAATCATGATCAAAAATACTCCGGTACCGGCAAATC |
| | | CTCCGACGACTTTCAGCCCGGCCAAGTTTGCTTCATTTATCACTCAGTACTCCACTGG |
| | | ACAGGTCAGCGTGGAAATTGAGTGGGAGCTACAGAAAGAAAACAGCAAACGTTGGAAT |
| | | CCAGAGATTCAGTACACTTCCAACTACAACAAGTCTGTTAATGTGGACTTTACTGTAG |
| | | ACACTAATGGTGTTTATAGTGAACCTCGCCCTATTGGAACCCGGTATCTCACACGAAA |
| | | CTTGTAA |

1. A pharmaceutical composition comprising
a recombinant adeno-associated virus (rAAV) vector,
a buffer;
a salt;
a cryoprotectant; and
a surfactant,
wherein the pH of the pharmaceutical composition is from about 7.1 to about 8.1.

2. The composition of claim 1, wherein the concentration of the buffer is about 1 mM to about 100 mM, optionally about 20 mM.

3. The composition of claim 1, wherein the buffer is Tris.

4. The composition of claim 1, wherein the concentration of the salt is about 10 mM to about 200 mM, optionally about 100 mM.

5. The composition of claim 1, wherein the salt is magnesium chloride (MgCl₂).

6. The composition of claim 1, wherein the concentration of the cryoprotectant is about 1% (w/v) to about 10% (w/v), optionally about 4% (w/v).

7. The composition of claim 1, wherein the cryoprotectant is sucrose.

8. The composition of claim 1, wherein the concentration of the surfactant is about 0.002% (w/v) to about 0.2% (w/v), optionally about 0.02% (w/v).

9. The composition of claim 1, wherein the surfactant is poloxamer 188.

10. The composition of claim 1, comprising about 1E+11 vector genome (vg)/mL to about 1E+15 vg/mL or about 3.0E+11 vg/mL to about 3.0E+13 vg/mL of the rAAV vector.

11. The composition of claim 1, wherein the pH of the composition is about 7.3 to about 7.9, optionally about 7.6.

12. The composition of claim 1, wherein the viscosity of the composition is about 0.5 mPa to about 5 mPa, optionally about 1 mPa to about 1.5 mPa, or optionally about 1.19 mPa.

13. The composition of claim 1, wherein the density of the composition is about 0.5 g/cm³ to about 5 g/cm³, optionally about 1 g/cm³ to about 1.5 g/cm³, or optionally about 1.03 g/cm³.

14. The composition of claim 1, wherein the conductivity of the composition is about 1 mS/cm to about 40 mS/cm, optionally about 10 mS/cm to about 20 mS/cm, or optionally about 16.9 mS/cm.

15. The composition of claim 1, wherein the rAAV vector comprises a capsid polypeptide of an AAV serotype selected from the group consisting of AAV1, AAV2, AAV3, AAV3A, AAV3B, AAV4, AAV5, AAV6, AAV7, AAV8, AAV9, AAV10, AAVrh10, AAVrh74, AAV12, AAV2i8, NP4, NP22, NP66, AAVDJ, AAVDJ/8, AAVDJ/9, AAVLK03, AAV1.1, AAV2.5, AAV6.1, AAV6.3.1, AAV9.45, RHM4-1, RHM15-1, RHM15-2, RHM15-3/RHM15-5, RHM15-4, RHM15-6, AAV hu.26, AAV1.1, AAV2.5, AAV6.1, AAV6.3.1, AAV9,45, AAV2i8, AAV29G, AAV2,8G9, AVV-LK03, AAV2-TT, AAV2-TT-S312N, AAV3B-S312N, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14 and AAVHSC15, optionally wherein the capsid polypeptide is of the AAV3B serotype.

16. (canceled)

17. The composition of claim 1, wherein the rAAV vector comprises a rAAV vector genome comprising an ATP7B transgene, or fragment thereof.

18.-21. (canceled)

22. A pharmaceutical composition of claim 1, comprising
a recombinant adeno-associated virus (rAAV) vector;
about 20 mM Tris;
about 100 mM MgCl₂;
about 4% (w/v) sucrose; and
about 0.02% (w/v) poloxamer 188,
wherein the pH of composition is from about 7.1 to about 8.1.

23.-24. (canceled)

25. The composition of claim 22, wherein the rAAV vector comprises a vector genome comprising a transgene and one or more elements, optionally, wherein the transgene is ATP7B, or a fragment thereof, optionally, wherein the ATP7B transgene comprises a nucleic acid encoding a copper-transporting ATPase 2 with a deletion of metal binding sites (MBS) 1-4, and optionally wherein the ATP7B transgene comprises a nucleic encoding a copper transporting ATPase 2 comprising the amino acid sequence of SEQ ID NO: 1 or both.

26.-28. (canceled)

29. A pharmaceutical composition comprising
a recombinant adeno-associated virus (rAAV) vector comprising a capsid polypeptide of the AAV3B serotype;
about 14 mM to about 26 mM Tris;
about 70 mM to about 130 mM MgCl₂;
about 2.8% (w/v) to about 5.2% (w/v) sucrose; and
about 0.014% (w/v) to about 0.26% (w/v) poloxamer 188,
wherein the pH of the composition is from about 7.1 to about 8.1.

30. The pharmaceutical composition of claim 29, wherein the rAAV vector comprises a vector genome comprising a transgene.

31. A pharmaceutical composition comprising
a recombinant adeno-associated virus (rAAV) vector comprising a capsid polypeptide of the AAV3B serotype and a vector genome comprising a transgene encoding a copper transporting ATPase 2 protein or fragment thereof;
about 20 mM Tris;
about 100 mM MgCl₂;
about 4% (w/v) sucrose; and
about 0.02% (w/v) poloxamer 188,
wherein the pH of composition is from about 7.1 to about 8.1.

32.-35. (canceled)

36. A pharmaceutical composition comprising
a recombinant adeno-associated virus (rAAV) vector comprising a capsid polypeptide of the AAV3B serotype and a vector genome comprising a transgene encoding a copper transporting ATPase 2 comprising or consisting of the amino acid sequence of SEQ ID NO:1;
about 20 mM Tris;
about 100 mM MgCl₂;
about 4% (w/v) sucrose; and
about 0.02% (w/v) poloxamer 188,
wherein the pH of composition is from about 7.1 to about 8.1.

37.-38. (canceled)

39. The composition of claim 1, wherein the pharmaceutical composition is lyophilized or is not lyophilized.

40. A vial comprising 0.5 mL to 10 mL of the composition of claim 1.

41. (canceled)

42. A method of treating a disease comprising administering an effective amount of the pharmaceutical composition of claim 1 to a human subject having the disease.

43. (canceled)

44. The method of claim 42, wherein the pharmaceutical composition is administered intravenously.

45. (canceled)

46. The method of claim 42, wherein the disease is due to a deficiency or dysfunction of copper-transporting ATPase 2, optionally wherein the disease is Wilson disease.

47. A method of reducing hepatic copper in a human subject in need thereof, comprising administering intravenously to the human subject the pharmaceutical composition of claim 1, optionally wherein the human subject has Wilson disease.

48. A method of treating Wilson disease in a human subject in need thereof, comprising administering intravenously to a human subject the pharmaceutical composition of claim 1.

49.-52. (canceled)

53. A kit comprising a pharmaceutical composition of claim 1, and instructions for use.
